(19) United States
(12) Patent Application Publication
(10) Pub. No.: US 2026/0216287 A1
(43) Pub. Date: Jul. 30, 2026

(54) SILK PEPTIDES ORAL FORMULATIONS AND METHODS OF THEIR USE

(71) Applicant: SILKLYFE INC., Palm Beach, FL (US)

(72) Inventors: Mike SANTOS, Fairhaven, MA (US); Charles KOPPEL, London (GB)

(21) Appl. No.: 19/346,763

(22) Filed: Oct. 1, 2025

Related U.S. Application Data
(63) Continuation of application No. 19/093,997, filed on Mar. 28, 2025, now abandoned.
(60) Provisional application No. 63/571,617, filed on Mar. 29, 2024.

(51) Int. Cl. A61K 38/17; A61K 47/69; A61P 1/00; A61P 1/04; A61P 1/16; A61P 11/00; A61P 29/00; A61P 37/04; A61P 39/06

(52) U.S. Cl. CPC A61K 38/1767; A61K 47/6951; A61P 1/00; A61P 1/04; A61P 1/16; A61P 11/00; A61P 29/00; A61P 37/04; A61P 39/06

(57) ABSTRACT

A composition comprising fragments of silk fibroin peptides for use in the treatment of a disorder or condition associated with a digestive, pulmonary, or skin disorder. The composition may be also used prophylactically. Several embodiments are proposed in which the composition may be orally administered in a form which prevents unintended degradation of the composition until it reaches a specific point of interest. The composition can be modified to include additional agents for use in the treatment of various disorders of the digestive system, the pulmonary system, or the skin as described herein.

### CROSS-REFERENCE TO RELATED APPLICATIONS

This application is a Continuation of U.S. application Ser. No. 19/093,997, filed on Mar. 28, 2025, which claims benefit of provisional Application No. 63/571,617, filed on Mar. 29, 2024. The entire contents of the above-identified applications are hereby fully incorporated herein by reference.

### BACKGROUND OF THE DISCLOSURE

Silk is a naturally occurring polymer. Most silk fibers are derived from silkworm moth (Bombyx mori) cocoons and include silk fibroin and sericin proteins. Silk fibroin is a fibrous material that forms a polymeric matrix bonded together with sericin. In nature, silk is formed from a concentrated solution of these proteins that are extruded through silkworm spinnerets to produce a highly insoluble fiber. These fibers have been used for centuries to form threads used in garments and other textiles. Humans have long appreciated silk for its lustrous appeal and remarkable physical properties, yet as modern uses of silk are explored, it becomes apparent that this outstanding biopolymer is more than textile fiber, but additionally a biopolymer having medicinal properties.

Many properties of silk make it an attractive candidate for products serving a variety of industries. Polymer strength and flexibility has supported classical uses of silk in textiles and materials, while silk biocompatibility has gained attention more recently for applications in the fields of medicine and agriculture.

Although a variety of products and uses related to silk are being developed, there remains a need for methods of producing and processing silk and silk-based products that can meet the demands of modern medicine. Additionally, there remains a need for silk-based products that can leverage silk polymer strength, flexibility, and biocompatibility to meet needs in the fields of medicine, agriculture, and material sciences. The present disclosure addresses these needs by providing methods for producing and processing silk as well as formulations of silk-based products useful in a variety of therapeutic applications. Silk in its solubilized aqueous form has been investigated for a range of therapeutic applications, including treatment of diabetes, See. Enzyme Hydrolysis of Silk Fibroin and the Anti-diabetic Activity of the Hydrolysates (C. L. Hu, J. Y. Cui, F. Z. Ren, C. Peng, Int. J. Food Eng. 2008); chronic wounds, See. Fibroin and Sericin from Bombyx mori Silk Stimulate Cell Migration through Upregulation and Phosphorylation of c-Jun (C. Martinez-Mora, A. Mrowiec, E. M. Garcia-Vizcaino, A. Alcaraz, J. L. Cenis, F. J. Nicolas, PLoS One 2012, 7, e42271) and inflammation See. Enhancement of Anti-Inflammatory Activity of PEP—1-FK506 Binding Protein by Silk Fibroin Peptide (D. W. Kim, H. S. Hwang, D. S. Kim, S. H. Sheen, D. H. Heo, G. Hwang, S. H. Kang, H. Kweon, Y. Y. Jo, S. W. Kang, K. G. Lee, J. Park, W. S. Eum, Y. J. Cho, S. Y. Choi, J. Microb. Biotechnol. 2012, 22, 494). Additional studies have investigated the utility of regenerated silk fibroin solutions in preclinical animal models for the treatment of ocular conditions, including dry eye, See. Effects of silk fibroin in murine dry eye (C. E. Kim, J. H. Lee, Y. K. Yeon, C. H. Park, J. Yang, Sci. Rep. 2017, 7, 44364). While silk has shown potential in a variety of therapeutics indications, silk fibroin, when taken in oral form, is thought to be destroyed by stomach acids. Therefore, the use of silk fibroin, maybe dependent upon formulations that avoid this degradation and allow for direct administration to the tissues of interest.

### SUMMARY OF THE DISCLOSURE

In some embodiments, the present disclosure provides silk-based product (SBP) formulations that comprise processed silk fibroin peptides with optionally one or more excipients, wherein the processed silk fibroin peptides comprise or is derived from natural or synthetic sources. The SBP formulation may comprises or may be combined with one or more members selected from the group consisting of: (a) a therapeutic agent; (b) a cargo; (c) a microorganism; and (d) a biological system The processed silk fibroin peptides and/or other SBP component (excipient, therapeutic agent, microbe, cargo, and/or biological system) may be present in SBP formulations at a concentration (by weight, volume, or concentration) of from about 0.0001% to about to about 99.9%, or greater than 99.9%.

The processed silk fibroin peptides and/or other SBP components include formulations of various silk-based product formulations and related methods of preparation and processing for uses in fields of human therapeutics and veterinary medicine as set forth in WO 2020/247594 entitled: Silk-Based Products, Formulations, and Methods of Use, the contents of which are incorporated in their entirety. Additional formulations and methods or their preparation of SBP formulations are set forth in U.S. Pat. Nos. 11,633,455 and 12,024,538, the contents of which are incorporated in their entirety.

The SBP formulation may have processed silk fibroin peptides and/or other SBP components (excipient, therapeutic agent, microbe, cargo, and/or biological system) present at a concentration of from about 0.01 pg/mL to about 200 mg/mL.

The SBP formulation may have processed silk fibroin peptides and/or other SBP components (excipient, therapeutic agent, microbe, cargo, and/or biological system) present at a concentration of from about 0.01 pg/mL to about 1 pg/mL, from about 0.05 pg/mL to about 2 pg/mL, from about 1 pg/mL to about 5 pg/mL, from about 2 pg/mL to about 10 pg/mL, from about 4 pg/mL to about 16 pg/mL, from about 5 pg/mL to about 20 pg/mL, from about 8 pg/mL to about 24 pg/mL, from about 10 pg/mL to about 30 pg/mL, from about 12 pg/mL to about 32 pg/mL, from about 14 pg/mL to about 34 pg/mL, from about 16 pg/mL to about 36 pg/mL, from about 18 pg/mL to about 38 pg/mL, from about 20 pg/mL to about 40 pg/mL, from about 22 pg/mL to about 42 pg/mL, from about 24 pg/mL to about 44 pg/mL, from about 26 pg/mL to about 46 pg/mL, from about 28 pg/mL to about 48 pg/mL, from about 30 pg/mL to about 50 pg/mL, from about 35 pg/mL to about 55 pg/mL, from about 40 pg/mL to about 60 pg/mL, from about 45 pg/mL to about 65 pg/mL, from about 50 pg/mL to about 75 pg/mL, from about 60 pg/mL to about 240 pg/mL, from about 70 pg/mL to about 350 pg/mL, from about 80 pg/mL to about 400 pg/mL, from about 90 pg/mL to about 450 pg/mL, from about 100 pg/mL to about 500 pg/mL, from about 0.01 ng/mL to about 1 ng/mL, from about 0.05 ng/mL to about 2 ng/mL, from about 1 ng/mL to about 5 ng/mL, from about 2 ng/mL to about 10 ng/mL, from about 4 ng/mL to about 16 ng/mL, from about 5 ng/mL to about 20 ng/mL, from about 8 ng/mL to about 24 ng/mL, from about 10 ng/mL to about 30 ng/mL, from about 12 ng/mL to about 32 ng/mL, from about 14 ng/mL to about 34 ng/mL, from about 16 ng/mL to about 36 ng/mL, from about 18 ng/mL to about 38 ng/mL, from about 20 ng/mL to about 40 ng/mL, from about 22 ng/mL to about 42 ng/mL, from about 24 ng/mL to about 44 ng/mL, from about 26 ng/mL to about 46 ng/mL, from about 28 ng/mL to about 48 ng/mL, from about 30 ng/mL to about 50 ng/mL, from about 35 ng/mL to about 55 ng/mL, from about 40 ng/mL to about 60 ng/mL, from about 45 ng/mL to about 65 ng/mL, from about 50 ng/mL to about 75 ng/mL, from about 60 ng/mL to about 240 ng/mL, from about 70 ng/mL to about 350 ng/mL, from about 80 ng/mL to about 400 ng/mL, from about 90 ng/mL to about 450 ng/mL, from about 100 ng/mL to about 500 ng/mL, from about 0.01 μg/mL to about 5 μg/mL, from about 2 μg/mL to about 10 μg/mL, from about 4 μg/mL to about 16 μg/mL, from about 5 μg/mL to about 20 μg/mL, from about 8 g/mL to about 24 μg/mL, from about 10 μg/mL to about 30 μg/mL, from about 12 μg/mL to about 32 μg/mL, from about 14 μg/mL to about 34 μg/mL, from about 16 μg/mL to about 36 g/mL, from about 18 μg/mL to about 38 μg/mL, from about 20 μg/mL to about 40 μg/mL, from about 22 μg/mL to about 42 μg/mL, from about 24 μg/mL to about 44 μg/mL, from about 26 g/mL to about 46 μg/mL, from about 28 μg/mL to about 48 μg/mL, from about 30 μg/mL to about 50 μg/mL, from about 35 μg/mL to about 55 μg/mL, from about 40 μg/mL to about 60 g/mL, from about 45 μg/mL to about 65 μg/mL, from about 50 μg/mL to about 75 μg/mL, from about 60 μg/mL to about 240 μg/mL, from about 70 μg/mL to about 350 μg/mL, from about 80 g/mL to about 400 μg/mL, from about 90 μg/mL to about 450 μg/mL, from about 100 μg/mL to about 500 μg/mL, from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 35 mg/mL to about 55 mg/mL, from about 40 mg/mL to about 60 mg/mL, from about 45 mg/mL to about 65 mg/mL, from about 50 mg/mL to about 75 mg/mL, from about 60 mg/mL to about 240 mg/mL, from about 70 mg/mL to about 350 mg/mL, from about 80 mg/mL to about 400 mg/mL, from about 90 mg/mL to about 450 mg/mL, from about 100 mg/mL to about 500 mg/mL, from about 0.01 g/mL to about 1 g/mL, from about 0.05 g/mL to about 2 g/mL, from about 1 g/mL to about 5 g/mL, from about 2 g/mL to about 10 g/mL, from about 4 g/mL to about 16 g/mL, or from about 5 g/mL to about 20 g/mL.

The SBP formulation may have processed silk fibroin peptides and/or other SBP components (excipient, therapeutic agent, microbe, cargo, and/or biological system) present in SBPs at a concentration of from about 0.01 pg/kg to about 1 pg/kg, from about 0.05 pg/kg to about 2 pg/kg, from about 1 pg/kg to about 5 pg/kg, from about 2 pg/kg to about 10 pg/kg, from about 4 pg/kg to about 16 pg/kg, from about 5 pg/kg to about 20 pg/kg, from about 8 pg/kg to about 24 pg/kg, from about 10 pg/kg to about 30 pg/kg, from about 12 pg/kg to about 32 pg/kg, from about 14 pg/kg to about 34 pg/kg, from about 16 pg/kg to about 36 pg/kg, from about 18 pg/kg to about 38 pg/kg, from about 20 pg/kg to about 40 pg/kg, from about 22 pg/kg to about 42 pg/kg, from about 24 pg/kg to about 44 pg/kg, from about 26 pg/kg to about 46 pg/kg, from about 28 pg/kg to about 48 pg/kg, from about 30 pg/kg to about 50 pg/kg, from about 35 pg/kg to about 55 pg/kg, from about 40 pg/kg to about 60 pg/kg, from about 45 pg/kg to about 65 pg/kg, from about 50 pg/kg to about 75 pg/kg, from about 60 pg/kg to about 240 pg/kg, from about 70 pg/kg to about 350 pg/kg, from about 80 pg/kg to about 400 pg/kg, from about 90 pg/kg to about 450 pg/kg, from about 100 pg/kg to about 500 pg/kg, from about 0.01 ng/kg to about 1 ng/kg, from about 0.05 ng/kg to about 2 ng/kg, from about 1 ng/kg to about 5 ng/kg, from about 2 ng/kg to about 10 ng/kg.

The SBP formulation may have processed silk fibroin peptides alone and/or other SBP components (excipient, therapeutic agent, microbe, cargo, and/or biological system) present in SBPs at a concentration of from about 0.01 pg/kg to about 10 mg/kg per kg of patient.

The processed silk fibroin peptides of the SBP formulation may comprise silk fibroin at a concentration between 0.01% and 100%. In one illustrative embodiment, the silk fibroin is present at a concentration of 0.5%. In one aspect, the silk fibroin is present at a concentration of 1%. In one aspect, the silk fibroin is present at a concentration of 2.5%. In one aspect, the silk fibroin is present at a concentration of 3%. In one aspect, the silk fibroin is present at a concentration of 5%. In one aspect, the Silk Fibroin is present at a concentration ranging from 0.01-20%. In another aspect, the silk fibroin is present at a concentration ranging from 0.01-100%.

The SBP formulation may be in powder form or in a solution which may be, but is not limited to, saline, phosphate buffer, borate buffer, and phosphate buffered saline. The solution may further comprise propylene glycol, sucrose and/or trehalose. Propylene glycol may be present in a concentration of 1%. Sucrose may be present in a concentration such as, but not limited to, 10 mM, 50 mM, 100 mM and 150 mM. Trehalose may be present in a concentration such as, but not limited to, 10 mM, 50 mM, 100 mM and 150 mM.

The SBP may be formulated, and the formulation may be as hydrogels, powders, suspensions, nanoparticles (solid, semi-solid, or gel), and solutions. The silk fibroin peptide concentration in the solution may be below 1% (w/v). The SBP may be a solution, and the SBP may be stressed. The SBP may be a hydrogel, and the SBP may be stressed. The SBP may be stressed by one or more methods which includes heating the SBP to 60° C. and autoclaving the SBP. The SBP may be a solution, and the solution may shear thin. The solutions may have the viscosity of a gel at a lower shear rate. The solutions may have the viscosity of a fluid at higher shear rates. The SBP may be formulated for topical administration. The SBP may be formulated for oral administration. The SBP may be formulated for inhalable administration. The SBP may be formulated for rectal administration.

In some embodiments, the present disclosure provides a method of preparing the SBP formulations comprising: (a) preparing the processed silk fibroin peptides, wherein the processed silk fibroin peptides comprise or are derived from natural or synthetic sources; and (b) preparing the SBP formulation using the processed silk fibroin peptides. In some embodiments, the present disclosure provides a method of treating inflammation. In other embodiments, the present disclosure provides a method and composition for gastric health. In further embodiments, the present disclosure provides a method and composition for treating liver health, the present disclosure provides a method for treating pulmonary health, the present disclosure provides a method for treating immune health.

The SBP may be administered orally in the form of enteric coated capsules, gel-caps, oil in water suspension and tablets, in an emulsion, in a hydrogel, or in buffered solutions. Without being bound to any particular theory, it is thought that silk fibroin peptides may still have therapeutic activity after digestion through the stomach (acid hydrolysis and pepsin digestion). However, others have reported https://www.fda.gov/media/156758/download page 26 section 6.2.1 as well as pages 15-26) that silk fibroin peptides are degraded in the stomach.

In one illustrative embodiment, formulations are envisioned to avoid digestion by stomach acids and enzymes. This would involve the use of powders, solutions, or gels of silk fibroin peptides encapsulated in an enteric coating. This could be in tablet, capsule, or coated powder form, and would allow for dissolution and delivery of the full-length silk fibroin peptides directly to the cells of interest. In one illustrative embodiment, EUDRACAP™ are used which are pre-locked hydroxypropyl methylcellulose (HPMC) hard capsules with a functional coating (facilitating a specific disintegration time). This could also be in suppository or enema form, and would allow for direct delivery to the lower gastric system.

Additionally other active compounds or active pharmaceutical ingredients could be added to the formulation. It is further completed within the scope of the disclosure that GRAS excipients or nanoparticulate formulations could be used to help the silk peptides penetrate the mucous layer in the lower gastric area. It's contemplated within the scope of the disclosure that active ingredients, ayurvedics can be co-administered producing a combinatorial or synergistic effect. This could include surfactants, penetration enhancers, polymeric and solid lipid nanoparticle formulations to prevent silk fibroin peptide degradation and/or the degraded silk fibroin peptides/amino acids have a similar anti-inflammatory effect.

In one illustrative embodiment the silk fibroin peptides, according to the disclosure, are presented in a salt form that may include but are not limited to the following salt forms: hydrochloride, sodium, sulfate, acetate, phosphate or diphosphate, chloride, potassium, maleate, calcium, citrate, mesylate, nitrate, tartrate, aluminum, zinc, and gluconate.

In an illustrative embodiment silk fibroin peptides according to the disclosure exhibit anti-inflammatory activity by inhibiting cytokines IL-6, IL-8 and TNF-α in human lung epithelial cells. This anti-inflammatory activity of silk fibroin peptides, alone or in combination with other therapeutic actives, can be use in therapeutically effective amounts to treat respiratory disorders that include but are not limited to pneumonia, acute respiratory distress syndrome (ARDS), asthma, chronic obstructive pulmonary disease (COPD), SARS-CoV, SARS-CoV-2, MERS-CoV, rhinitis, bronchitis, emphysema, common cold, influenza, histoplasmosis, RSV, tuberculosis, whooping cough, autoimmune diseases (Lupus, etc), cystic fibrosis and pulmonary sarcoidosis.

Silk fibroin peptides, alone or in combination with other therapeutic actives, can be used in therapeutically effective amounts as a protective or decontaminating composition to a fluid, structure or tissue selected from the group consisting of internal coatings/inclusions, lung fluid, lung/alveolar surfaces, and lung tissues. Silk fibroin peptides, alone or in combination with other therapeutic actives, can deactivate, substantially deactivate, or facilitate inflammation removal due to irritant gases or reactive pollutant combustion gases for individuals in need.

Irritant gases or reactive pollutant combustion gases may be selected from the group consisting of Sulfur dioxide, Sulfur trioxide, nitric oxide, nitrogen dioxide, nitrous oxide, hydrogen Sulfide, compounds containing a thiol group, compounds containing a monosulfide group, compounds containing a hydrosulfide group, compounds containing a cyanide group and compounds containing polysulfides, or the like.

In a further illustrative embodiment silk fibroin peptides according to the disclosure exhibit anti-inflammatory activity by inhibiting cytokines IL-6, IL-8 and RANTES in human colorectal cells. This anti-inflammatory activity of silk fibroin peptides, alone or in combination with other therapeutic actives, can be use in therapeutically effective amounts to treat colorectal disorders that include but are not limited to: inflammatory bowel disease (IBD), Crohn's disease, ulcerative colitis, other autoimmune diseases, and other types of colitis e.g. microscopic colitis/lymphocytic colitis. Silk fibroin peptides, alone or in combination with other therapeutic actives, can also be used as a coating or to protect and/or decontaminate a fluid, structure, or tissue selected from a group consisting of: internal coatings, inclusions, colorectal fluid, colorectal surfaces, colorectal tissue, or the like. Silk fibroin peptides described herein may also deactivate, substantially deactivate, or facilitate the reduction or removal of inflammation.

In another illustrative embodiment silk fibroin peptidesaccording to the disclosure exhibit anti-inflammatory activity by inhibiting cytokines IL-8, MIP-1-α and RANTES in human gastric cells. This anti-inflammatory activity of silk fibroin peptides, alone or in combination with other therapeutic actives, can be use in therapeutically effective amounts to treat gastric disorders that include but are not limited to gastritis, bacterial colonization (Helicobacter pylori), NSAID side effects, and autoimmune diseases (autoimmune gastritis). Silk fibroin peptides, alone or in combination with other therapeutic actives, can also be used as a coating or to protect and/or decontaminate a fluid, structure, or tissue selected from a group consisting of: internal coatings, inclusions, colorectal fluid, colorectal surfaces, colorectal tissue, or the like. Silk fibroin peptidesdescribed herein may also deactivate, substantially deactivate, or facilitate the reduction or removal of inflammation.

In a further illustrative embodiment silk fibroin peptidesaccording to the disclosure exhibit anti-inflammatory activity by inhibiting cytokines in murine splenocytes/BMDCs/human monocytes—i.e. systemic anti-inflammatory activity. This anti-inflammatory activity of Silk Fibroin proteins can be used in therapeutically effective amounts to treat progressive endothelial dysfunction, platelet sludging, blood clots, stroke, CVD, cancer, diabetes mellitus, chronic kidney disease, non-alcoholic fatty liver disease, autoimmune disorders (Lupus, etc.), neurodegenerative disorders (Alzheimer's etc), sarcopenia, osteoporosis, immunosenescence, and chronic inflammatory systemic diseases (e.g. rheumatoid arthritis, multiple sclerosis).

In another illustrative embodiment silk fibroin peptides according to the disclosure exhibit cytoprotective effect on cell function. This cytoprotective effect of silk fibroin peptides against oxidative stress in lung cells (restoration of cell viability, antiapoptotic effect) can be used in therapeutically effective amounts to treat asthma, COPD, and lung cancer. This cytoprotective effect of silk fibroin peptides can be used against oxidative stress in liver cells resulting in restoration of cell viability and antiapoptotic effect. Silk fibroin peptides can be used in therapeutically effective amounts to treat alcoholic liver disease, non-alcoholic steatohepatitis and viral hepatitis. This cytoprotective effect of silk fibroin peptides against oxidative stress in gastric cells (restoration of cell viability, antiapoptotic effect) and can be used in therapeutically effective amounts to treat gastroduodenal ulcers, GI malignancies (cancers), and IBD.

In a further illustrative embodiment silk fibroin peptides according to the disclosure exhibit an immunostimulatory effect by increasing levels of cytokines in murine splenocytes/BMDCs/human monocytes. This immunostimulatory of silk fibroin peptides can be used in therapeutically effective amounts to treat infections (any that benefit from increased cytokine response), cancer, and immunodeficiency disorders.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-IC show anti-inflammatory activity by inhibition of cytokines in Human lung epithelial cells (A549)-IL-6.

FIGS. 2A-2C show anti-inflammatory activity by inhibition of cytokines in Human lung epithelial cells (A549)-IL-8.

FIGS. 3A-3C show anti-inflammatory activity by inhibition of cytokines in Human lung epithelial cells (A549)-TNF-α.

FIGS. 4A-4C show anti-inflammatory activity by inhibition of cytokines in Human colorectal cells (HT-29)-IL-6.

FIGS. 5A-5C show anti-inflammatory activity by inhibition of cytokines in Human colorectal cells (HT-29)-IL-8.

FIGS. 6A-6C show anti-inflammatory activity by inhibition of cytokines in Human colorectal cells (HT-29)-RANTES.

FIGS. 7A-7C show anti-inflammatory activity by inhibition of cytokines in Human gastric cells (HT-29)-IL-8.

FIGS. 8A-8C show anti-inflammatory activity by inhibition of cytokines in Human gastric cells (HT-29)-MIP-1-α.

FIGS. 9A-9C show anti-inflammatory activity by inhibition of cytokines in Human gastric cells (HT-29)-RANTES.

FIGS. 10A-10C show anti-inflammatory activity by inhibition of cytokines in Human gastric cells (HT-29)-TNF-α.

FIGS. 11A-11C show cytoprotective effect against oxidative stress in Human lung cells (A549) by restoration of cell viability.

FIGS. 12A-12C show cytoprotective effect against oxidative stress in Human lung cells (A549) by anti-apoptotic effect (JC-1).

FIGS. 13A-13C show cytoprotective effect against oxidative stress in Human lung cells (A549) by anti-apoptotic effect (cell cycle).

FIGS. 14A-14C show cytoprotective effect against oxidative stress in Human liver cells (HepG2) by restoration of cell viability.

FIGS. 15A-15F show cytoprotective effect against oxidative stress in Human liver cells (HepG2) by anti-apoptotic effect (JC-1).

FIGS. 16A-16C show cytoprotective effect against oxidative stress in Human liver cells (HepG2) by anti-apoptotic effect (cell cycle).

FIGS. 17A-17C show cytoprotective effect against oxidative stress in Human gastric cells (AGS) by restoration of cell viability.

FIGS. 18A-18C show cytoprotective effect against oxidative stress in Human gastric cells (AGS) by anti-apoptotic effect (JC-1).

FIGS. 19A-19C show cytoprotective effect against oxidative stress in Human gastric cells (AGS) by anti-apoptotic effect (ROS).

FIGS. 20A-20C show immunostimulatory activity-increase in levels of cytokines in Murine splenocytes-TNF-α.

FIGS. 21A-21C show immunostimulatory activity-increase in levels of cytokines in Murine splenocytes-IL-6.

FIGS. 22A-22C show immunostimulatory activity-increase in levels of cytokines in Murine splenocytes-IFN-γ.

FIGS. 23A-23C show immunostimulatory activity-increase in levels of cytokines in Murine splenocytes-MIP-1-α.

FIGS. 24A-24C show immunostimulatory activity-increase in levels of cytokines in Murine BMDCs-TNF-α.

FIGS. 25A-25C show immunostimulatory activity-increase in levels of cytokines in Murine BMDCs-IL-6.

FIGS. 26A-26C show immunostimulatory activity-increase in levels of cytokines in Murine BMDCs-IFN-γ.

FIGS. 27A-27C show immunostimulatory activity-increase in levels of cytokines in Murine BMDCs-MIP-1-α.

FIGS. 28A-28C show immunostimulatory activity-increase in levels of cytokines in Murine BMDCs-IL-1-β.

FIGS. 29A-29C show immunostimulatory activity-increase in levels of cytokines in Human monocytes (THP-1)-TNF-α.

FIGS. 30A-30C show immunostimulatory activity-increase in levels of cytokines in Human monocytes (THP-1)-IL-6.

FIGS. 31A-31C show immunostimulatory activity-increase in levels of cytokines in Human monocytes (THP-1)-IL-8.

FIGS. 32A-32C show immunostimulatory activity-increase in levels of cytokines in Human monocytes (THP-1)-MIP-1-α.

FIGS. 33A-33C show immunostimulatory activity-increase in levels of cytokines in Human monocytes (THP-1)-IL-1-β.

FIGS. 34A-34C show anti-inflammatory activity-inhibition of cytokines in Murine splenocytes-TNF-α.

FIGS. 35A-35C show anti-inflammatory activity-inhibition of cytokines in Murine splenocytes-IL-6.

FIGS. 36A-36C show anti-inflammatory activity-inhibition of cytokines in Murine splenocytes-IFN-γ.

FIGS. 37A-37C show anti-inflammatory activity-inhibition of cytokines in Murine splenocytes-MIP-1-α.

FIGS. 38A-38C show anti-inflammatory activity-inhibition of cytokines in Murine splenocytes-IL-1-β.

FIGS. 39A-39C show anti-inflammatory activity-inhibition of cytokines in Murine BMDCs-TNF-α.

FIGS. 40A-40C show anti-inflammatory activity-inhibition of cytokines in Murine BMDCs-IL-6.

FIGS. 41A-41C show anti-inflammatory activity-inhibition of cytokines in Murine BMDCs-IFN-γ.

FIGS. 42A-42C show anti-inflammatory activity-inhibition of cytokines in Murine BMDCs-MIP-1-α.

FIGS. 43A-43C show anti-inflammatory activity-inhibition of cytokines in Murine BMDCs-IL-1-β.

FIGS. 44A-44C[[44a-44c]] show anti-inflammatory activity-inhibition of cytokines in Human monocytes (THP-1)-TNF-α.

FIGS. 45A-45C show anti-inflammatory activity-inhibition of cytokines in Human monocytes (THP-1)-IL-6.

FIGS. 46A-46C show anti-inflammatory activity-inhibition of cytokines in Human monocytes (THP-1)-IL-8.

FIGS. 47A-47C show anti-inflammatory activity-inhibition of cytokines in Human monocytes (THP-1)-MIP-1-α.

FIGS. 48A-48C show anti-inflammatory activity-inhibition of cytokines in Human monocytes (THP-1)-IL-1-β.

### DETAILED DESCRIPTION

Embodiments of the present disclosure relate to silk-based products (SBPs), formulations and their methods of use. The term “silk” generally refers to a fibrous material formed by insects and some other species that includes tightly bonded protein filaments. Herein, the term “silk” is used in the broadest sense and may embrace any forms, variants, or derivatives of silk peptides.

Silk fibers from silkworm moth (Bombyx mori) cocoons include two main components, sericin (usually present in a range of 20-30%) and silk fibroin (usually present in a range of 70-80%). Structurally, silk fibroin forms the center of the silk fibers and sericin acts as the gum coating the fibers. Sericin is a gelatinous protein that holds silk fibers together with many of the characteristic properties of silk (see Qi et al. (2017) Int J Mo! Sci 18:237 and Deptuch et al. (2017) Materials 10:1417, the contents of each of which are herein incorporated by reference in their entireties). Silk fibroin is an insoluble fibrous protein consisting of layers of antiparallel beta sheets. Its primary structure mainly consists of recurrent serine, alanine, and glycine repeating units. The isoelectric point of silk fibroin has been determined to be around 4.2. Silk fibroin monomers include a complex of heavy chain (around 350 kDa) and light chain (around 25 kDa) protein components. Typically, the chains are joined by a disulfide bond. With some forms, heavy chain and light chain segments are non-covalently bound to a glycoprotein. Polymers of silk fibroin monomers may form through hydrogen bonding between monomers, typically increasing mechanical strength (see Qi et 7. (2017) Int J Mo! Sci 18:237). During silk processing, fragments of silk fibroin monomers may be produced, including, but not limited to, fragments of heavy and/or light chains. These fragments may retain the ability to form hydrogen bonds with silk fibroin monomers and fragments thereof. Herein, the term “silk fibroin” is used in its broadest sense and embraces silk fibroin peptides, silk fibroin polymers, silk fibroin monomers, silk fibroin heavy and light chains, silk fibroin fragments, and variants, derivatives, or mixtures thereof from any of the wild type, genetically modified, or synthetic sources of silk described herein. The present disclosure includes methods and formulations utilizing processed silk fibroin peptides and SBPs, different forms of SBP formulations, and a variety of applications for utilizing processed silk fibroin peptides, SBPs, and SBP formulations alone or in combination with various compounds and compositions.

In order to produce the desired products, the basic components of the invention as described above may be combined with other cosmetic and pharmaceutical ingredients which are well known to cosmetic and pharmaceutical chemists.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art, cobalamin for deactivation of reactive compounds; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may also be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934@). Lubricants, glidants, flavors, coloring agents and stabilizers may also be added for ease of fabrication and use.

The SBP may include from about 0.0001% to about 100% (w/v) of silk fibroin peptides. In an aspect, fibroin peptides are produced by providing raw silk (e.g., unpurified silk such as silk yarn), the raw silk comprising fibers containing silk fibroin and sericin. First, the raw silk is degummed in a salt solution, specifically a sodium carbonate solution with a sodium carbonate concentration of 0.02 to 0.5 M sodium carbonate at a temperature of about 60 to about 100° C., and for a time of greater than 60 minutes to about 480 minutes. In a preferred aspect, degumming is performed in 0.5 M sodium carbonate at 85° C. for either 240 or 360 minutes. In an aspect, degumming provides degummed silk fibers having a sericin concentration of 0-0.5 wt %. After the degumming, the silk fibroin fibers are further processed by dissolving, preferably in aqueous solution. Dissolving preferably includes using 5M to 13M lithium bromide for 1 hour to overnight at 50° C. to 100° C. to provide dissolved silk fibroin peptides, or dissolving the degummed silk fibers using a mixture of calcium chloride, ethanol, and water in a molar ratio of 1:2:8 for 1 hour to overnight at 50° C. to 100° C. to provide dissolved silk fibroin peptides. In a specific aspect, 10 wt % to 20 wt % silk fibroin is dissolved in 9.3M lithium bromide at about 60° C. for 16 hours (overnight). In another specific aspect, 10 wt % to 20 wt % silk fibroin is dissolved in a mixture of calcium chloride, ethanol, and water in a molar ratio of 1:2:8 at 80° C. for 2 hours.

Solvents used to dissolve processed silk fibroin may include a buffer. In some embodiments, solvent used is an organic solvent. Organic solvents include, but are not limited to hexafluoroisopropanol (HFIP), methanol, isopropanol, ethanol, or combinations thereof. In some embodiments, solvents include a mixture of an organic solvent and water or an aqueous solution. Solvents may include water or aqueous solutions. Aqueous solutions may include aqueous salt solutions that include one or more salts. Such salts may include but are not limited to lithium bromide (LiBr), lithium thiocyanate, Ajisawa's reagent, a chaotropic agent, calcium nitrate, or other salts capable of solubilizing silk, including any of those disclosed in U.S. Pat. No. 9,623,147 (the content of which is herein incorporated by reference in its entirety). In some embodiments, solvents used in processed silk solutions include high salt solutions. Ajisawa's reagent comprises a mixture of calcium chloride, ethanol, and water in a molar ratio of 1:2:8 respectively.

In one illustrative embodiment, the degummed silk fibroin is dissolved in 5 to 13 M LiBr. The concentration of LiBr may be 9.3 M. Dissolving in LiBr can be done at 60° C. for 16 hours (overnight).

In some embodiments, solvents used in processing silk solutions may include Ajisawa's reagent, as described in Zheng et al. (2016) Journal of Biomaterials Applications 31:450-463, the content of which is herein incorporated by reference in its entirety.

After the silk fibroin fibers are dissolved, they can be diluted prior to further purification. In an aspect, the dissolved silk fibers are diluted in water to provide a concentration of 5 to 20% w/v silk fibroin fibers. Optionally the diluted fibroin solution is filtered through a polypropylene, polyethersulfone, nylon, or cellulose, diatomaceous earth, perlite depth prefilter to remove particulates and provide a clarified silk fibroin peptide solution.

The diluted silk fibroin peptide solution can be purified using dialysis, diafiltration, or tangential flow filtration (TFF) using a regenerated cellulose or polyethersulfone filter and concentrating and recovering processed silk fibroin from the TFF. For example, the silk solution is concentrated 2×, and then diafiltered to remove the chaotropic agents, i.e., lithium bromide/calcium chloride and ethanol. Diafiltration can be performed against water at pH 3.0-11.0, salt solution, i.e., sodium chloride, potassium chloride, (10 mM-500 mM) at pH 3.0-11.0, buffer, i.e., sodium phosphate, potassium phosphate, tromethamine, at 10 mM-250 mM at pH 3.0-11.0, or buffer containing 10 mM-500 mM salt pH 3.0-11.0. Diafiltration is performed for 5-15 diavolumes, or until a sufficient amount of the chaotropic agent is removed. Silk fibroin peptides may also be purified according to the methods set forth in U.S. Pat. No. 12,024,538.

In an aspect, molecular weight, specifically number average molecular weight is determined by size exclusion chromatography. In an aspect, the molecular weight of the processed silk fibroin peptides is up to about 350 kDa, but more particularly about 20 to about 50 kDa. For most applications, the molecular weight is 25-42 kDa. For ocular solutions, the molecular weight is 25-50 kDa. After purification, the silk fibroin peptides are referred to as processed silk fibroin peptides or purified silk fibroin peptides and SBP.

The SBP may include one or more excipients. The one or more excipients may include one or more of sucrose, lactose, phosphate salts, sodium chloride, potassium phosphate monobasic, potassium phosphate dibasic, sodium phosphate dibasic, sodium phosphate monobasic, polysorbate 80, phosphate buffer, phosphate buffered saline, sodium hydroxide, sorbitol, mannitol, lactose USP, Starch 1500, microcrystalline cellulose, potassium chloride, sodium borate, boric acid, sodium borate decahydrate, magnesium chloride hexahydrate, calcium chloride dihydrate, sodium hydroxide, Avicel, dibasic calcium phosphate dehydrate, tartaric acid, citric acid, fumaric acid, succinic acid, malic acid, hydrochloric acid, polyvinylpyrrolidone, copolymers of vinylpyrrolidone and vinylacetate, hydroxypropylcellulose, hydroxyethylcellulose, hydroxypropylmethylcellulose, polyvinyl alcohol, polyethylene glycol, acacia, trehalose, and sodium carboxymethylcellulose.

One or more of the excipients may include phosphate buffer. One or more of the excipients may include phosphate buffered saline. One or more of the excipients may include sucrose. The excipients may include boric acid, sodium borate decahydrate, sodium chloride, potassium chloride, magnesium chloride hexahydrate, calcium chloride dihydrate, sodium hydroxide, and hydrochloric acid. The SBP may include at least one excipient selected from one or more members of the group consisting of sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremophor EL), cremophor RH 40, cremophor RH 60, dalpha-tocopherol, PEG 1000 succinate, polysorbate 20, polysorbate 80, solutol HS 15, sorbitan monooleate, poloxamer-407, polox-amer-188, Labrafil M-1944CS, Labrafil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cotton-seed oil, olive oil, peanut oil, peppermint oil, safflower oil, sesame oil, soybean oil, hydrogenated vegetable oils, hydrogenated soybean oil, medium chain triglycerides of coconut oil, medium chain triglycerides of palm seed oil, beeswax, d-alpha-tocopherol, oleic acid, medium-chain monoglycerides, medium-chain di-glycerides, alpha-cyclodextrin, beta-cyclodextrin, hydroxypropyl-beta-cyclodextrin, sulfobutyletherbeta-cyclodextrin, hydrogenated soy phosphatidylcholine, distearoylphosphatidylglycerol, L-alpha dimyristoylphosphatidylcholine, L-alpha-dimyristoylphosphatidyl glycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS15, sorbitan monooleate (Span 20), sulfobutyletherbeta-cyclodextrin, transcutol, triacetin, 1-dodecylazacyclo-heptan-2-one, caprolactam, castor oil cottonseed oil, ethyl acetate, medium chain triglycerides, methyl acetate, oleic acid, safflower oil, sesame oil, soybean oil, tetrahydrofuran, glycerin, and PEG 4 kDa.

Further contemplated herein are pharmaceutical compositions comprising one or more of the silk fibroin peptide compositions and a pharmaceutically acceptable carrier. Such pharmaceutical compositions may be made using any technique generally known in the art.

As a non-limiting example, a method of making a pharmaceutical composition includes mixing one or more of the silk fibroin peptide compositions with a pharmaceutically acceptable carrier. For example, the method of making a pharmaceutical composition can include mixing silk fibroin peptide compositions under sterile conditions with a pharmaceutically acceptable carrier with preservatives, buffers, and/or propellants to create the pharmaceutical composition.

In a further illustrative embodiment, pharmaceutical compositions including silk fibroin peptide compositions may be made as follows: one or more of the silk fibroin peptide compositions, as the active ingredient, is intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The compositions according to the disclosure can be in unit dosage forms such as tablets, pills, capsules, powders, granules, ointments, sterile parenteral solutions or suspensions, metered aerosol or liquid sprays, drops, ampules, enemas, auto-injector devices or suppositories, for oral parenteral, intranasal, sublingual or rectal administration, or for administration by inhalation or insufflation.

One or more silk fibroin peptide compositions according to the disclosure can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of a silk fibroin peptide composition or an amount effective to treat a disease, may be determined initially from the Examples described herein and adjusted for specific targeted diseases using routine methods.

In some illustrative embodiments, processed silk fibroin peptides or SBPs according to the disclosure are prepared to provide anti-inflammatory properties, as evidenced by the examples that follow. In some illustrative embodiments processed silk fibroin peptides or SBPs may be administered to a human subject alone or in combination with other active pharmaceutical ingredients to provide therapeutic a therapeutic effect dose to produce anti-inflammatory effects. It is contemplated within the scope of the disclosure that processed silk fibroin peptides or SBPs alone or in combination with other therapeutic agents may be used to treat various inflammatory diseases. Without being bound to any particular theory, it is thought that silk fibroin peptides according to the disclosure greatly reduces/inhibits the production of inflammatory cytokines IL-6, IL-8, and TNF-a. It is further thought that silk fibroin peptides according to the disclosure improve cell viability and reduce cell death in the presence of oxidative stress/damage leading to a cytoprotective effect.

The compositions according to the disclosure can be administered orally, nasally, rectally, intracisternally, intraperitoneally, transdermally (as by powders, ointments, or drops), and/or parenterally. As used herein, “parenteral” administration refers to modes of administration other than through the gastrointestinal tract, which include intravenous, intramuscular, intraperitoneal, intrasternal, intramammary, intraocular, intrapulmonary, intrathecal, subcutaneous and intraarticular injection and infusion. Surgical implantation may also be contemplated, including, for example, embedding a composition of the disclosure in the body such as, for example, in a tissue, in the abdominal cavity, under the splenic capsule, brain, or in the cornea.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono-or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

Accordingly, the route of administration can include intranasal administration, oral administration, inhalation administration, subcutaneous administration, transdermal administration, intradermal administration, intra-arterial administration with or without occlusion, intracranial administration, intraventricular administration, intravenous administration, buccal administration, intraperitoneal administration, intraocular administration, intramuscular administration, implantation administration, topical administration, intratumor administration, and/or central venous administration.

The therapeutically-effective amount of a compound of the present invention may be determined by one of ordinary skill in the art, and includes exemplary dosage amounts for a mammal of from about 0.05 to 1000 mg/kg; 1-1000 mg/kg; 1-50 mg/kg; 5-250 mg/kg; 250-1000 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like.

In one aspect of the instant disclosure, it is contemplated within the scope of the disclosure that delivery of the silk fibroin peptides would involve an inhalable form of silk fibroin. This could either be an inhalable powder or inhalable liquid. It's further contemplated within the scope of the disclosure that the silk fibroin peptide may be in powder form without any additives, or the silk fibroin peptide may be chemically or physically attached to a carrier compound and optionally other pharmaceutical excipients allowing for its inhalation. Some of the delivery methods for this could include inhalers, nebulizers, humidifiers, vaporizers, etc. The simplest formulation would be just the silk ingredient; however, other ingredients or excipients could be used to increase efficacy. These may include excipients to aid in nebulization or improve inhalation of the formula or excipients or formulation approaches (nanoparticulate, etc.) to improve delivery, as set forth in “Challenges and Strategies to Enhance the Systemic Absorption of Inhaled Peptides and Proteins” See. (https.//link.springer.com/article/10.1007/s11095-022-03435-3), the teachings of which are incorporated herein.

### Processing and Purification of Silk Fibroin Peptides

The processed silk fibroin peptides and/or other SBP components include methods of processing and purification as set forth in WO 2023/251264 A1 entitled: Methods for reducing impurities in silk fibroin preparations, the contents of which are incorporated in their entirety.

In an aspect, a method of purifying a silk fibroin peptide comprises preparing an aqueous silk fibroin solution having a concentration of greater than or equal to 5% w/v silk fibroin from the silk fibroin preparation, wherein the silk fibroin preparation comprises a chaotropic salt; and exchanging the chaotropic salt from the silk fibroin solution at a pH below the isoelectric point of the silk fibroin, wherein the pH is between 2 and 5, replacing the chaotropic salt with a buffer comprising 10 to 300 mM of a second salt, or a combination thereof, to prepare the purified silk fibroin.

Described herein are methods of reducing impurities, particularly elemental impurities introduced during purification, of silk fibroin peptides. During purification of silk fibroin which has been prepared by a process including dissolution in lithium bromide or another chaotropic salt as is standard in the art, the inventors have found that standard methods using tangential flow filtration (TFF) with water only in the retentate/replacement feed or dialysis provide a final material that has much higher lithium than bromide, typically 500-3000 ppm Li and 20-200 ppm Br, normalized to the amount of silk fibroin. This is unexpected, as it would be expected that both Li and Br would be completely or almost completely removed during exhaustive TFF or dialysis. In addition, as the mass of Br is 11.6 times greater than that of lithium one would expect that the Br residuals would be 11.6 times greater than Li, not 100-fold less.

For example, U.S. Pat. No. 9,517,191 and related patents claim that silk fibroin preparations have 0 ppm to 500 ppm of “inorganic residuals”, such as lithium bromide residuals of 10 ppm to 300 ppm, “measurable using a high performance liquid chromatography lithium bromide assay”. However, while Example 5 describes a TFF process to remove lithium bromide, the specification does not provide any data demonstrating removal of the lithium and bromide to the levels of 10 ppm to 300 ppm. Further, the '191 patent does not disclose if the level of inorganic residuals is normalized to the amount of silk fibroin in solution, which would be necessary. For example, any solution could be diluted with DI water to lower the level of residuals to below 300 ppm in solution but would not demonstrate any improved removal or residuals as compared to the amount of silk. Further, as shown in the examples herein, repeating the examples of the '191 patent provides a silk fibroin material with 1000-2000 ppm Li and 40-100 ppm Br, normalized to the amount of silk fibroin. The '191 patent does not provide a pH or salt composition for the TFF solution purported to remove Li and Br residuals.

The inventors have unexpectedly found that using a retentate having a pH of 3 to 4.5, such as pH 4, and/or using a TFF replacement feed solution of a salt concentration of 10 to 300 mM NaCl, for example, resulted in a dramatic decrease in Li levels, specifically 20-500 ppm normalized to the amount of silk fibroin. The Br levels in these same samples can be 200-1500 ppm normalized to the amount silk fibroin. The reduction in Li levels is particularly important for product safety in pharmaceuticals and consumer products, for example. If one wanted to maintain the safe level of Li in a pharmaceutical product comprised of dried or concentrated silk fibroin in order to utilize the benefits of larger amounts of silk, these methods could be employed to ensure more complete removal of elemental impurities.

In an aspect, a method of purifying a silk fibroin peptides comprises preparing an aqueous silk fibroin solution having a concentration of greater than or equal to 5% w/v silk fibroin from the silk fibroin preparation, wherein the silk fibroin preparation comprises a chaotropic salt; and exchanging the chaotropic salt from the silk fibroin solution at a pH below the isoelectric point of the silk fibroin, wherein the pH is between 2 and 5, replacing the chaotropic salt with a buffer comprising 10 to 300 mM of a second salt, or a combination thereof, to prepare the purified silk fibroin.

Raw silk starting material can be obtained from the silkworm species Bombyx mori. Other examples of silk producer species include, but are not limited to, Bombyx mandarina, Bombyx sinesis, Anaphe moloneyi, Anaphe panda, Anaphe reticulate, Anaphe ambrizia, Anaphe carteri, Anaphe venata, Anapha infracta, Antheraea assamensis, Antheraea assama, Antheraea mylitta, Antheraea pernyi, Antheraea yamamai, Antheraea polyphemus, Antheraea oculea, Anisota senatoria, Apis mellifera, Araneus diadematus, Araneus cavaticus, Automeris io, Atticus atlas, Copaxa multifene strata, Coscinocera hercules, Callosamia promethea, Eupackardia calleta, Eurprosthenops australis, Gonometa postica, Gonometa rufobrunnea, Hyalophora cecropia, Hyalophora euryalus, Hyalophora gloveri, Miranda auretia, Nephila madagascarensis, Nephila clavipes, Pachypasa otus, Pachypasa atus, Philosamia ricini, Pinna squamosa, Rothschildia hesperis, Rothschildia lebeau, Sarnia Cynthia, and Sarnia ricini.

In an aspect, the silk fibroin preparation comprises a chaotropic salt. As used herein, a chaotropic salt is a salt that disrupts the structure of macromolecules, such as silk fibroin.

In an aspect, the method comprises preparing an aqueous silk fibroin solution having a concentration of greater than or equal to 5% w/v silk fibroin from the silk fibroin preparation, wherein the silk fibroin preparation was prepared by a process comprising dissolving silk fibroin fibers in 5M to 13 M LiBr.

In an aspect, fibroin is produced by providing raw silk (e.g., unpurified silk such as silk yarn, cocoons), the raw silk comprising fibers containing silk fibroin and sericin. First, the raw silk is degummed in a salt solution, specifically a sodium carbonate solution with a sodium carbonate concentration of 0.05 to 1 M, specifically 0.1 to 1 M, more specifically 0.2 to 0.5 M sodium carbonate at a temperature of about 60 to about 90° C., and for a time of greater than 60 minutes to about 480 minutes. In a preferred aspect, degumming is performed in 0.5 M sodium carbonate at 85° C. for either 240 or 360 minutes. In an aspect, degumming provides degummed silk fibers having a sericin concentration of 0-0.5 wt %.

Of particular relevance to the present application, most prior art processes for purifying silk fibroin use 0.02 M sodium carbonate with boiling for 30 or 60 minutes to provide degummed silk fibroin. The inventors have found that this prior art process produces a material that is not favorable for subsequent processing steps, specifically TFF performed with a concentration of 5 to 20% w/v silk fibroin fibers. The prior art degumming process provides silk fibroin that is so viscous in solution it cannot be run at concentrations higher than about 1% w/v by TFF. Without being held to theory, it is believed that the molecular weight, polydispersity, and/or distribution of molecular weights of silk fibroin produced by prior art degumming processes is unfavorable for subsequent processing steps.

After the degumming, the silk fibroin fibers are further processed by dissolving, preferably in an aqueous solution comprising a chaotropic agent. Exemplary chaotropic agents include lithium bromide, lithium chloride, calcium chloride, ethanol, guanidinium chloride, and urea. Dissolving preferably includes using 5M to 13M lithium bromide for 1 hour to overnight at 50° C. to 100° C. to provide dissolved silk fibers, or dissolving the degummed silk fibers using a mixture of calcium chloride, ethanol, and water in a molar ratio of 1:2:8, respectively, for 1 hour to overnight at 50° C. to 100° C. to provide dissolved silk fibers. In a specific aspect, 10 wt % to 20 wt % silk fibroin is dissolved in 9.3M lithium bromide at about 60° C. for 16 hours (overnight). In an aspect, using the TFF/Dialysis methods described herein the purified silk fibroin peptide comprises 10 to 600 ppm lithium per mg silk. In another aspect, the purified silk fibroin peptide comprises 10 to 600 ppm bromine per mass of silk fibroin.

In another aspect, the silk fibroin peptide is prepared by a process comprising dissolving the degummed silk fibers using a mixture of calcium chloride, ethanol, and water in a molar ratio of 1:2:8 for 1 hour to overnight at 50° C. to 100° C.

After the silk fibroin fibers are dissolved, they can be diluted prior to further purification. In an aspect, the dissolved silk fibers are diluted in water to provide a concentration of 5 to 20% w/v silk fibroin fibers. Optionally the diluted fibroin solution is filtered through a polypropylene, polyethersulfone, nylon, or cellulose, diatomaceous earth, perlite depth prefilter to remove particulates and provide a clarified silk fiber solution.

The aqueous silk fibroin solution having a concentration of greater than or equal to 5% w/v silk fibroin is then purified by and exchanging the chaotropic salt from the silk fibroin solution at a pH below the isoelectric point of the silk fibroin, wherein the pH is between 2 and 5, replacing the chaotropic salt with a buffer comprising 10 to 300 mM of a second salt, or a combination thereof, to prepare the purified silk fibroin.

In an aspect, the diluted silk fibroin fibers are then purified by tangential flow filtration (TFF) using, for example, continuous diafiltration by tangential flow filtration (TFF). Diafiltration is the fractionation process that washes smaller molecules through a membrane and leaves larger molecules in the retentate without significantly changing concentration. It can be used to remove salts or exchange buffers. It can remove ethanol or other small solvents or additives.

In continuous diafiltration, the diafiltration solution (water, buffer, or a salt solution) is added to the sample feed reservoir at the same rate as filtrate is generated. In this way the volume in the sample reservoir remains constant, but the small molecules (e.g., salts) that can freely permeate through the membrane are washed away in the filtrate (also called the permeate). Using salt removal as an example, each additional diafiltration volume (DV; also referred to herein as a diavolume) reduces the salt concentration further as the salt ions are removed in the filtrate. (A diafiltration volume is the volume of sample before the diafiltration solution is added.) Anything that isn't filtered out is the “retentate”. In the present case, the retentate includes the majority of the silk fibroin.

In the process described herein, in one method, the “sample”, also called the retentate, which includes the silk fibroin, is pH adjusted down to pH 2 to 5, for example, from its original pH of 8.5-9.

In an aspect, exchanging salt ions from the aqueous silk fibroin solution is by continuous diafiltration by tangential flow filtration (TFF) with a 5 kDa to 10 kDa molecular weight cut-off membrane by a process comprising providing a reduced pH retentate and filtering with at least three diafiltration volumes with a replacement feed of water, wherein the reduced pH retentate is a retentate comprising the silk fibroin and having a pH of 2 to 5. In an aspect, prior to providing the reduced pH retentate, the method comprises filtering least 3 diafiltration volumes, preferably at least 5 diafiltration volumes, with a water replacement feed.

The reduced pH retentate is a retentate comprising the silk fibroin and having a pH of 2 to 5, preferably 3 to 4.5, more preferably 3 to 4, and most preferably 4. In another aspect, exchanging salt ions from the aqueous silk fibroin solution is by dialysis against the buffer having a pH of 2-5, wherein a pH of 2-5 is maintained through at least a portion of the dialysis procedure, preferably through the entire dialysis procedure. [0024] In yet a further aspect, exchanging salt ions from the aqueous silk fibroin solution is by continuous diafiltration by tangential flow filtration (TFF) with a 5 kDa to 10 kDa molecular weight cut-off membrane by a process comprising filtering with at least three diafiltration volumes of a salt solution replacement feed, wherein the salt solution replacement feed comprises 10 to 300 mM of the salt. In an aspect, prior providing the salt solution replacement feed, the method comprises filtering least 3 diafiltration volumes, preferably at least 5 diafiltration volumes, with a water replacement feed.

In an aspect, the salt solution replacement feed, e.g., the second salt, comprises 10 to 300 mM of a Mg, Ca, K, or Na salt, specifically NaCl or CaCh, more specifically 150 mM NaCl. The pH of the salt solution replacement is not critical, but is preferably unbuffered, such as between pH 6 and 8.

In another aspect, exchanging salt ions from the aqueous silk fibroin solution is by dialysis in the buffer comprising 10 to 300 mM of the monovalent or divalent salt.

In another aspect, the method further comprises adjusting the pH of the purified silk fibroin preparation to a pH of 7-9, preferably 8.5-9.

In the following aspects, the silk fibroin preparation is prepared by a process comprising dissolving the degummed silk fibers using a mixture of calcium chloride, ethanol, and water in a molar ratio of 1:2:8 for 1 hour to overnight at 50° C. to 100° C.

In an aspect, exchanging salt ions comprises continuous diafiltration by tangential flow filtration (TFF), dialysis, or a combination thereof. In an aspect, the calcium ions are reduced to 10 to 500 ppm.

In this case, exchanging salt ions from the aqueous silk fibroin solution is by tangential flow filtration (TFF) with a 5 kDa to 10 kDa molecular weight cut-off membrane by a process comprising providing a reduced pH retentate and filtering with at least three diafiltration volumes with a replacement feed of water, wherein the reduced pH retentate is a retentate comprising the silk fibroin and having a pH of 2 to 5.

Alternatively, purifying the silk fibroin solution is by dialysis in the buffer having a pH of 2-5, wherein a pH of 2-5 is maintained through at least a portion of the dialysis procedure, preferably through the entire dialysis procedure.

In another alternative, exchanging salt ions from the aqueous silk fibroin solution is by continuous diafiltration by tangential flow filtration (TFF) with a 5 kDa to 10 kDa molecular weight cut-off membrane by a process comprising filtering with at least three diafiltration volumes of a salt solution replacement feed, wherein the salt solution replacement feed comprises 10 to 300 mM of the monovalent or divalent salt. [0033] In yet another alternative, exchanging salt ions from the aqueous silk fibroin solution is by dialysis in the buffer comprising 10 to 300 mM of the salt.

In any of the foregoing aspects, the silk fibroin preparation is prepared by degumming silk yarn in 0.05 to 1 M sodium carbonate at a temperature of about 60° C. to about 90° C., and for a time of greater than 60 minutes to about 480 minutes, to provide degummed silk fibers having a sericin concentration of 0-0.5 wt %.

Following TFF, the solution may be filtered through a ~0.8-2 μm polypropylene, polyethersulfone, nylon, or cellulose, diatomaceous earth, perlite depth filter and stored at either frozen to −80° C. or stored at 4° C., preferably at a silk fibroin concentration of 5% to 20% (w/v).

The silk fibroin peptides prepared by the foregoing method preferably has a weight average molecular weight of less than 90 kDa or less than as measured by size exclusion chromatography depending upon the method used, or less than 20 kDa as determined by dynamic light scattering. It is important to note that the determined molecular weight of silk fibroin preparations is highly dependent upon the method used to determine molecular weight. The silk fibroin prepared by the foregoing method also preferably has polydispersity of less than 1.4 as determined by dynamic light scattering.

### Examples

The invention is further illustrated by the following examples, which are intended to illustrate and not limit the invention. The characterization for the silk fibroin peptide used in the examples are identified as TF-134, TF-125 and TF-136. The TF-134 and TF-136 were prepared using the CaCP/EtOH/Water dissolution method, while the TF-125 was prepared using LiBr in water. The average molecular weight of the silk fibroin peptide was measured by ultra-performance liquid chromatography size exclusion chromatography (UPLC-SEC). A Waters Acquity H-Class UPLC equipped with a Waters Acquity UPLC Protein BEH SEC Column, 200 Å, 1.7 μm, 4.6 mm×150 mm and Waters Acquity tuneable ultraviolet (TUV) detector was used. Sample temperature was maintained at 4° C. throughout the analysis. An isocratic flow rate of 0.3 mL/min was run using a mobile phase consisting of 100 mM Tris-HCl with 400 mM sodium perchlorate at pH 8.0. Ultraviolet detection was monitored at 280 nm. Molecular weights were calculated using Waters BEH 200 Å Protein SEC Standard Mix.

These examples utilized silk solutions having varying concentrations and parameters as set forth in the table below:

| S. No. | Parameter | TF-125 | TF-134 | TF-136 |
|---|---|---|---|---|
| 1 | Name | Silk | Silk | Silk |
| | | Fibroin Solution | Fibroin Solution | Fibroin Solution |
| 2 | Strength | Ingredient | Ingredient | Ingredient |
| | | Strength | Strength | Strength |
| 3 | Concentration/ | 10.2% | 12.4% | 13.2% |
| | Content | (w/v) in water | (w/v) in water | (w/v) in water |
| 4 | Batch No. | TF-125 | TF-134 | TF-136 |
| 5 | Lot. No. | TF-125 | TF-134 | TF-136 |
| 6 | Physical | Clear, | Clear, | Clear, |
| | Description | yellow, slightly | yellow, slightly | yellow, slightly |
| | | viscous solution | viscous solution | viscous solution |
| 7 | Formulation | Aqueous | Aqueous | Aqueous |
| | Type | solution | solution | solution |
| 8 | COA | Available | Available | Available |
| 9 | MSDS | Available | Available | Available |
| 10 | Storage | −20° C. +/− | −20° C. +/− | −20° C. +/− |
| | condition | 10° C. | 10° C. | 10° C. |
| 11 | Mol wt | 42.6 kDa | 40.4 kDa | 38.3 kDa |
| 12 | pH | 8.8 | 9.0 | 8.5 |
| 13 | Solubility | Freely | Freely | Freely |
| | | soluble in water | soluble in water | soluble in water |
| 14 | Density | NA | NA | NA |
| 15 | Features | Precipitate | Precipitate | Precipitate |
| | indicating | is present in the | is present in the | is present in the |
| | deterioration | solution (no longer | solution (no longer | solution (no longer |
| | | clear) or solution | clear) or solution | clear) or solution |
| | | has gelled | has gelled | has gelled |
| 16 | Disposal | Non-hazardous. | Non-hazardous. | Non-hazardous. |
| | method | Can be disposed | Can be disposed | Can be disposed |
| | | of normally | of normally | of normally |

### A. Evaluation of Anti-Inflammatory Potential of Fibroin Proteins (TF-125, TF-134, TF-136) in Lung Inflammation, IBD/Colitis and Gastric Inflammation Using In Vitro Cells Based Assays

Inflammation is the process in which the body's immune system deals with infection, injury, and harmful substances. Inflammation is the body's response to insults, which include infection, trauma, and hypersensitivity. The inflammatory response is complex and involves a variety of mechanisms to defend against pathogens and repair tissue. Lung inflammation (pneumonitis) is caused by exposure to airborne toxins, pollutants, irritants, infections (bacteria, fungi or viruses), and diseases like asthma or bronchitis.

Symptoms of inflamed lungs include wheezing, breathing problems, and chest pain and tightness. During inflammation, numerous types of inflammatory cells are activated. Each releases cytokines and mediators to modify activities of other inflammatory cells. Orchestration of these cells and molecules leads to progression of inflammation. Clinically, acute inflammation is seen in pneumonia and acute respiratory distress syndrome (ARDS), whereas chronic inflammation is represented by asthma and chronic obstructive pulmonary disease (COPD). Because the lung is a vital organ for gas exchange, excessive inflammation can be life threatening. The lung is constantly exposed to harmful pathogens, an immediate and intense defense action (mainly inflammation) is required to eliminate the invaders as early as possible. Lung inflammation and cytokine storm also is pivotal target in SARS-COV-2 infection as well. The lung is the primary site of infection in viral disorders.

Pneumonitis is a general term for lung inflammation. It can cause difficulty breathing and is often accompanied by a cough. Acute lung inflammation that drives alveolar hemorrhage is driven primarily by the release of inflammatory cytokines and chemokines that result in stimulating an influx of neutrophils into the airways. Lung inflammation/Pneumonitis occurs when an irritating substance causes the tiny air sacs (alveoli) in your lungs to become inflamed. This inflammation makes it difficult for oxygen to pass through the alveoli into the bloodstream. Many irritants, ranging from airborne molds to chemotherapy drugs, have been linked to pneumonitis. Inflammation of the lungs is relevant in many inflammatory disorders such as COPD, asthma, rhinitis, bronchitis etc. A549 is a human lung alveolar cell line that is very well reported by researchers as an assay model to investigate the anti-inflammatory effects of test agents against numerous stimulants ranging from cytokines, LPS, allergens, pollutants, microbes etc. Inhibition of inflammatory cytokines in A549 induced by inflammatory stimulation is employed as an end point for determining of anti-inflammatory potential in lung inflammation conditions.

### STUDY DESIGN—Anti-Inflammatory Activity by Inhibition of Cytokines in Human Lung Epithelial Cells (A549)

### Study Design

Test System—Human Lung epithelial Cells (A549)
Number of cells plated—0.1 million cells/well in 24 Well plate
FBS concentration—0%,
Test items—TF-125 (10.2%), TF-134 (12.4%), TF-136 (13.2%)
Time points—24 h pre-treatment, 24 h stimulation (hu-TNF-α 10 ng/ml),
Estimation method—ELISA.
Positive Control—Dexamethasone, Quercetin

### Procedure

Cells were plated in 10% FBS in 24-well plates and incubated for 24 hours. Cells were then serum starved in 0% FBS for 24 hours and then were treated with 3 Test items in 0% FBS at non-cytotoxic concentrations for 24 hours. After incubation, cells were stimulated with inflammatory stimulus (hu-TNF-α 10 ng/ml). After 24 hours of stimulation, culture supernatants were collected. Levels of cytokines (IL-6, MIP-1α, IL-8, TNF-α, GMCSF) were determined using ELISA as follows: Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm.

### Calculations

Percent Inhibition in each sample was calculated with respect to (wrt) control (hu-TNFα treated cells). [(B−A)/B]*100; Where A=Test Item treated cells, B=Control (hu-TNFα treated) cells.

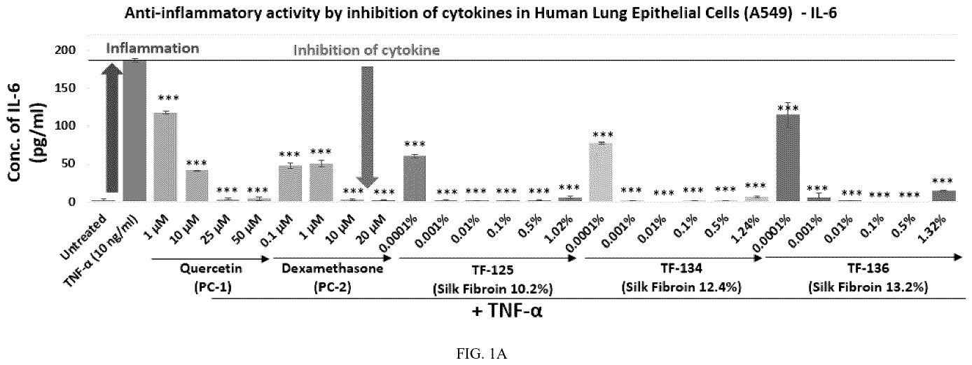

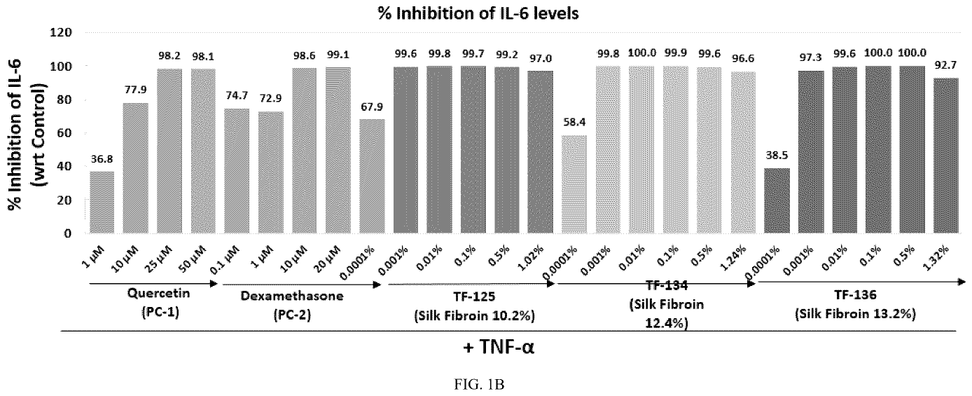

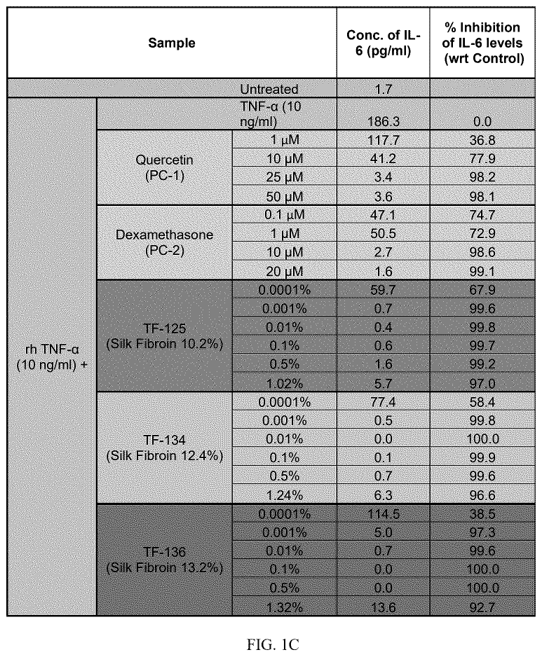

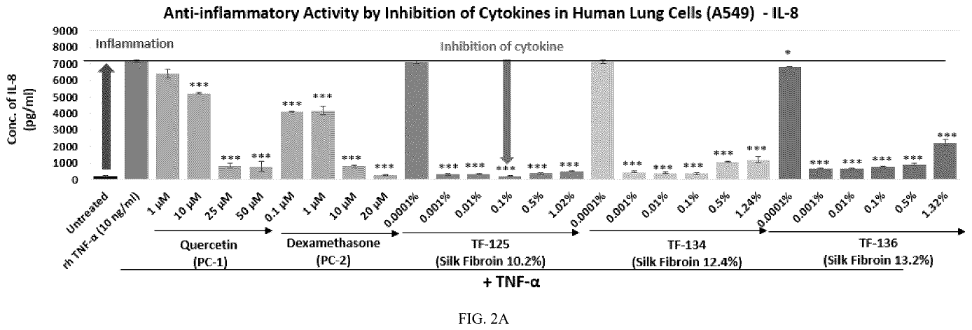

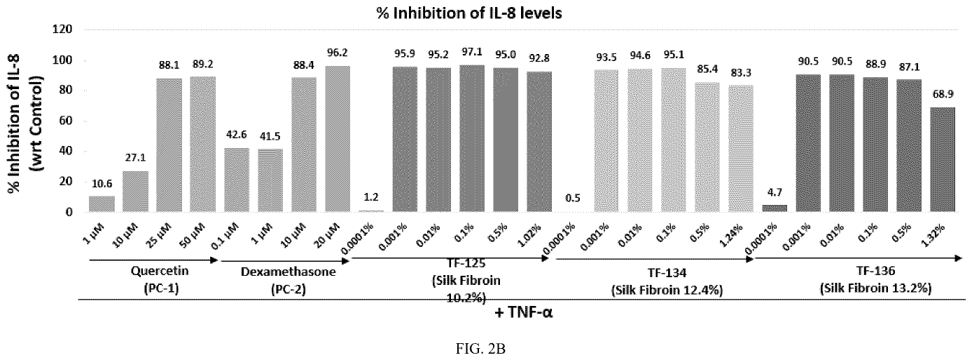

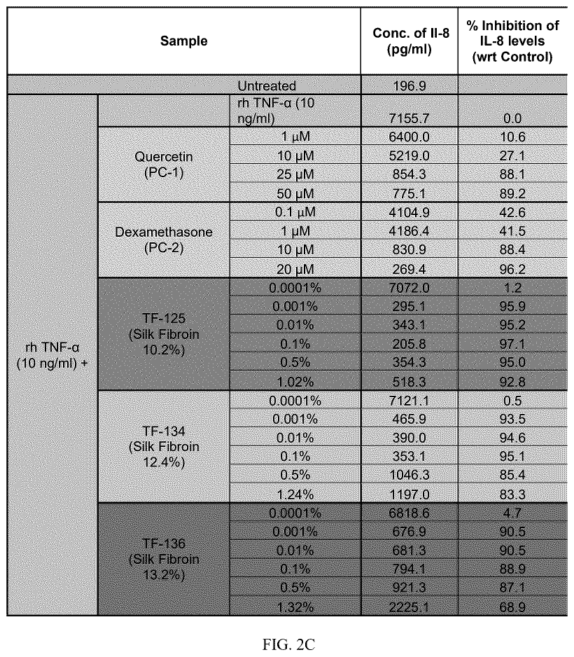

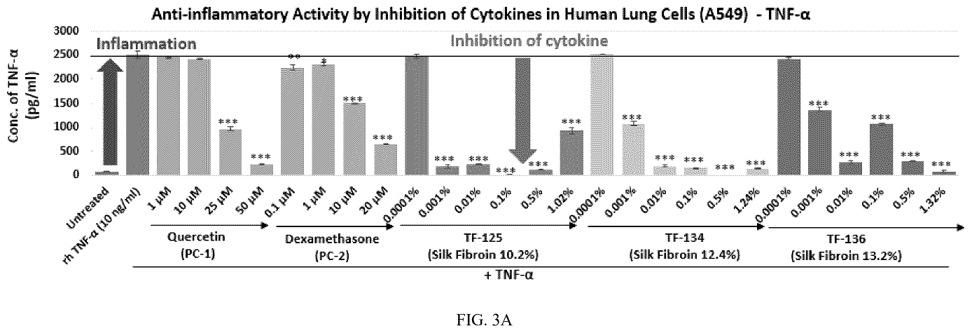

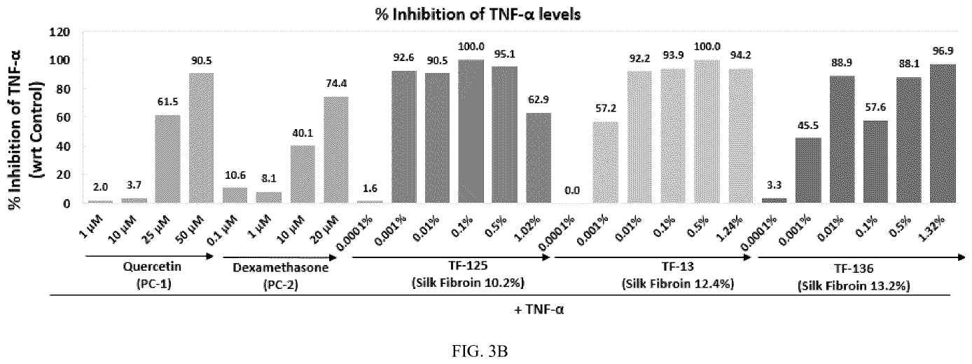

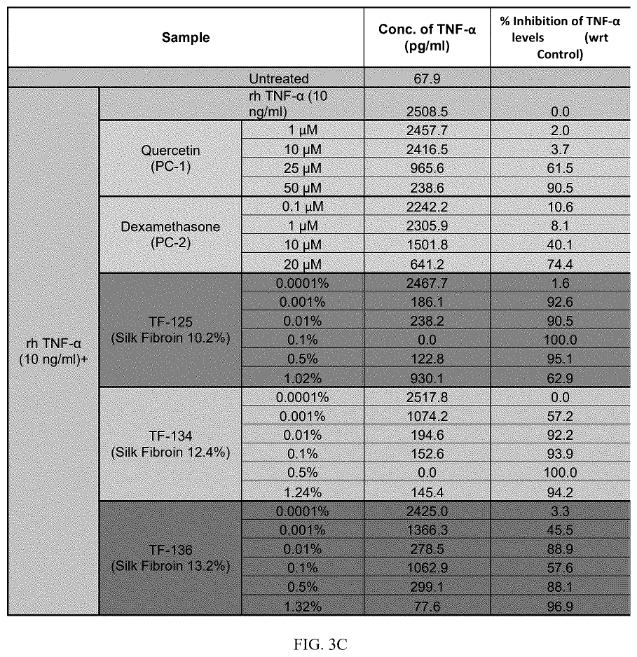

Results: seen at FIGS. 1-3.

Findings: Anti-inflammatory potential: Test items comprising silk fibroin ingredient greatly reduced the production of inflammatory cytokines IL-6, IL-8, and TNF-a as well or better than steroid control (dexamethasone). This data shows the anti-inflammatory potential for our silk fibroin peptides according to the disclosure. It should translate to a reduction in lung inflammation with direct treatment.

### B. Anti-Inflammatory Activity by Inhibition of Inflammatory Cytokines (IL-6, IL-8, IL-1-β, MIP-1-α, RANTES)

Inflammatory bowel disease (IBD) is a term that describes disorders involving long-standing (chronic) inflammation of tissues in your digestive tract. Types of IBD include: Ulcerative colitis. This condition involves inflammation and sores (ulcers) along the lining of the large intestine (colon) and rectum. Crohn's disease. This type of IBD is characterized by inflammation of the lining of the digestive tract, which often can involve the deeper layers of the digestive tract.

People with inflammatory bowel diseases (IBD) like Crohn's disease and ulcerative colitis have chronic intestinal inflammation. Symptoms include stomach cramps, diarrhea and gas. Medications and surgery can help manage IBD flares, putting the condition into remission.

Cytokines play an important role in the immunopathogenesis of inflammatory bowel disease (IBD), including Crohn's disease and ulcerative colitis, where they drive and regulate multiple aspects of intestinal inflammation. The imbalance between proinflammatory and anti-inflammatory cytokines that occurs in IBD results in disease progression and tissue damage and limits the resolution of inflammation. Targeting cytokines have been novel strategies in the treatment of IBD.

IBD stands for inflammatory bowel disease, which is an umbrella term used to describe disorders that cause chronic inflammation of the gastrointestinal (GI) tract. The two most common forms of IBD are Crohn's disease and ulcerative colitis. Ulcerative colitis is a long-term inflammatory condition that starts in the rectum and may spread to colon.

Since inflammation is the key hallmark of IBD, cytokines play an important role in the immunopathogenesis of inflammatory bowel disease (IBD), including Crohn's disease and ulcerative colitis. Mucosal and systemic concentrations of many pro- and anti-inflammatory cytokines are highly elevated in inflammatory bowel disease. An imbalance between proinflammatory and anti-inflammatory cytokines was found for the IL-1/IL-1ra ratio in the inflamed mucosa of patients with Crohn's disease, ulcerative colitis, diverticulitis, and infectious colitis. An inhibition of these cytokines certainly helps in curbing IBD and colitis.

Human colorectal cell line (HT-29) is well reported cell line used as model system to study anti-inflammatory effect of test agents by targeting inflammatory cytokines secretion. Inhibition of various pro-inflammatory cytokines and other markers is used as end point to claim anti-inflammatory potential in Colitis/IBD.

### Study Design

Test System—Human Colorectal Cells (HT-29)
Number of cells plated—0.1 million cells/well in 24 Well plate
FBS concentrations—0%,
Test items—TF-125 (10.2%), TF-134 (12.4%), TF-136 (13.2%)
Time points—24 h pre-treatment, 24 h stimulation (hu-TNF-α 20 ng/ml).
Estimation method—ELISA.
Positive Control—Sulfasalazine, EGCG

### Procedure

Cells were plated in 10% FBS in 24-well plates and incubated for 24 hours. Cells were then serum starved in 0% FBS for 24 hours. Cells were treated with 3 Test items in 0% FBS at various concentrations for 24 h. After incubation, cells were stimulated with inflammatory stimulus (hu-TNF-α 20 ng/ml). After 24 hours of stimulation, culture supernatants were collected. Levels of cytokines (IL-6, IL-8, MIP-1α, RANTES, IL-1β) were determined using ELISA as follows: Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm.

### Calculations

Percent Inhibition in each sample was calculated wrt control (hu-TNFα treated cells).

[(B−A)/B]*100; Where A=Test Item treated cells, B=Control (hu-TNFα treated) cells.

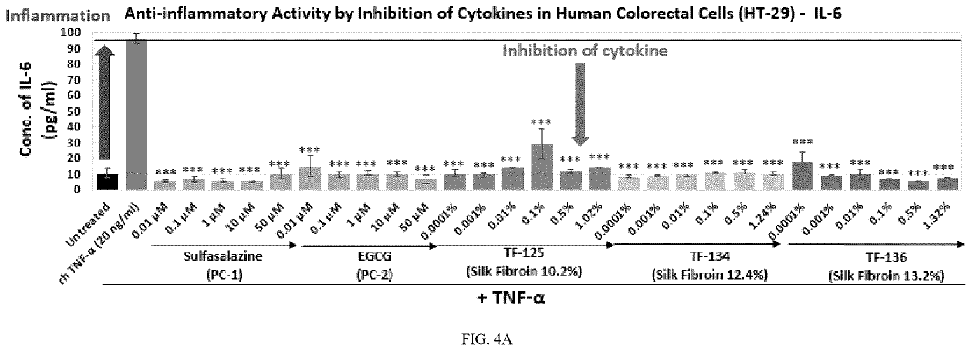

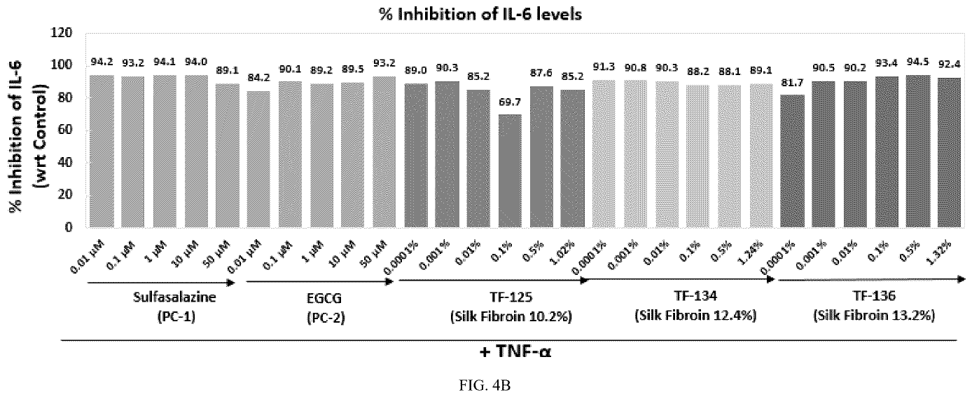

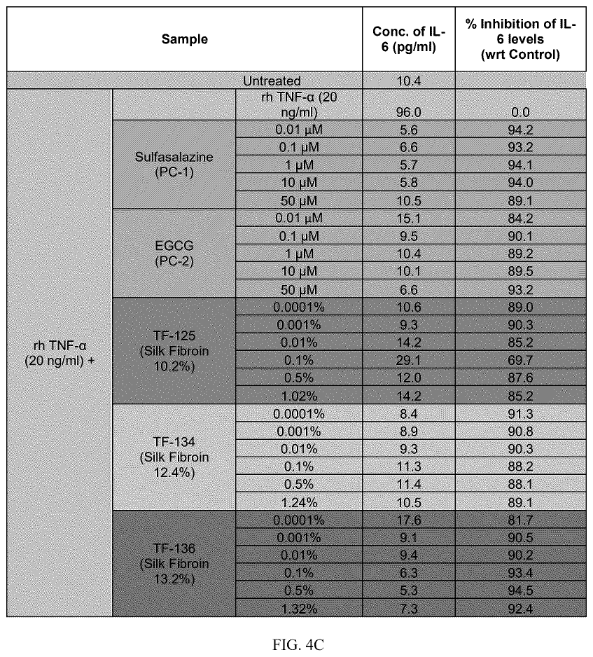

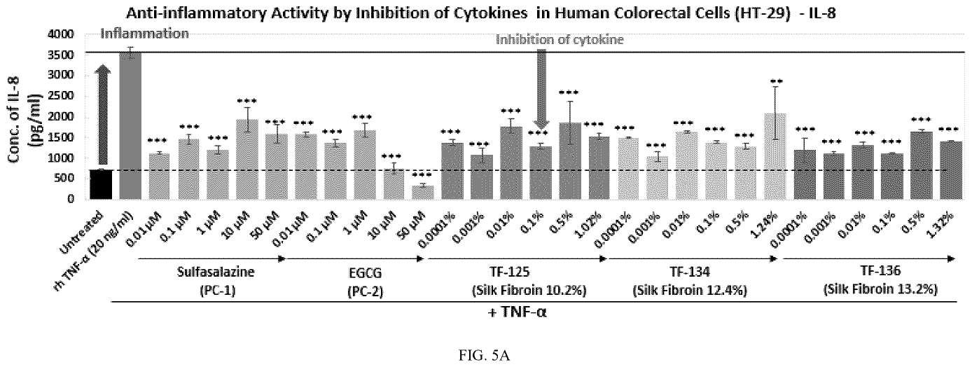

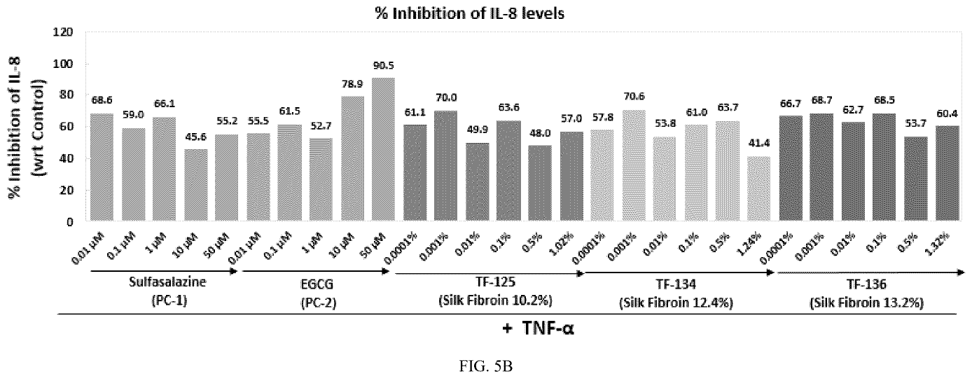

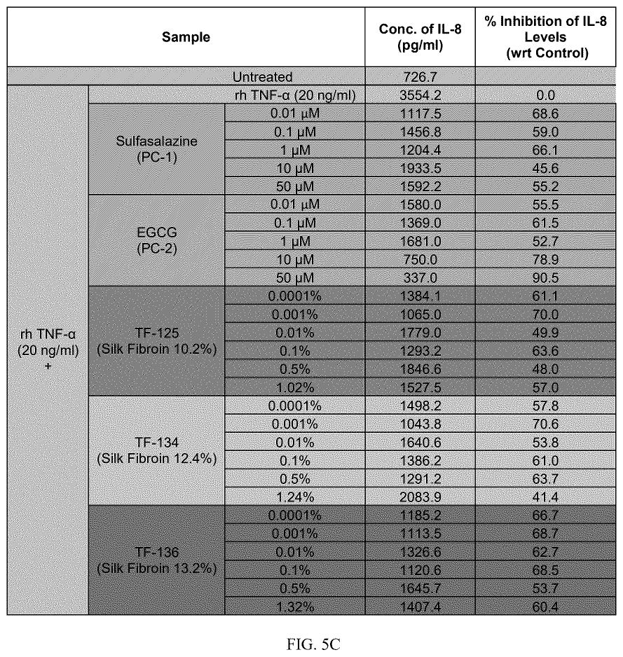

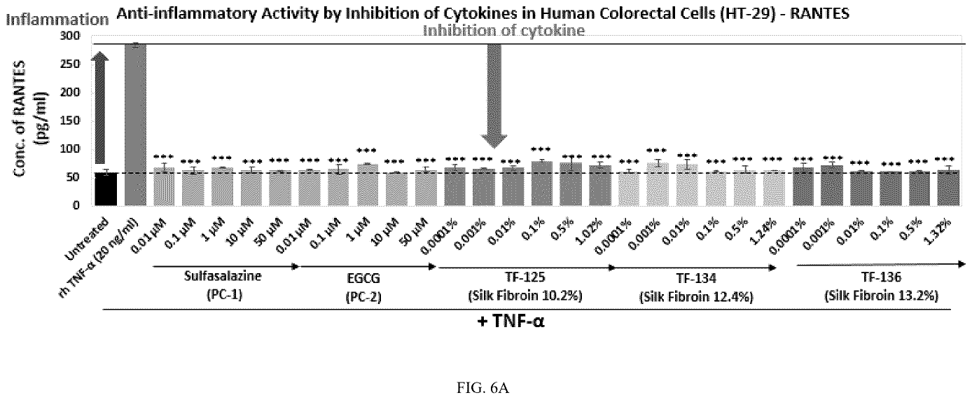

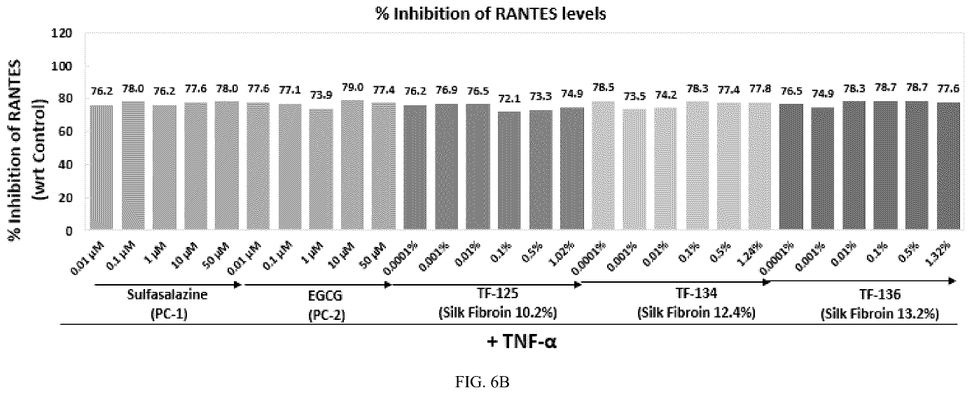

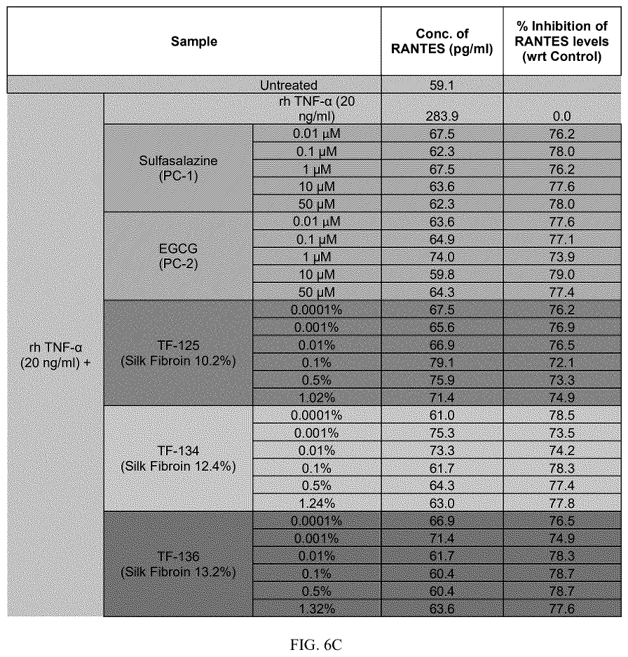

Results: seen at FIGS. 4-7.

IBD/Colitis model: Cell line HT-29 (Human colorectal cell line) stressed with human TNF-a (20 ng/mL) to induce inflammation

Test items comprising silk fibroin ingredient greatly reduced the production of inflammatory cytokines IL-6, IL-8, and RANTES as well or better than control (sulfasalazine; known treatment for IBD)

Findings: The data shows the anti-inflammatory potential for our silk fibroin ingredient. It should translate to a reduction in lower gastric cell (colorectal) inflammation upon direct treatment with the ingredient.

### C. GASTRIC INFLAMMATION: Anti-Inflammatory Activity by Inhibition of Inflammatory Cytokines (IL-6, IL-8, IL-1-β, MIP-1-α, RANTES)

Gastritis is a general term for a group of conditions with one thing in common: Inflammation of the lining of the stomach. The inflammation of gastritis is most often the result of infection with the same bacterium that causes most stomach ulcers or the regular use of certain pain relievers. Drinking too much alcohol also can contribute to gastritis. Gastritis may occur suddenly (acute gastritis) or appear slowly over time (chronic gastritis). In some cases, gastritis can lead to ulcers and an increased risk of stomach cancer.

Hyperinflammatory conditions with exaggerated expression of inflammatory cytokines exist in Gastritis. Inhibition of these inflammatory cytokines is helpful in managing the condition involving gastric inflammation. Gastritis is an inflammation, irritation, or erosion of the lining of the stomach. It can occur suddenly (acute) or gradually (chronic). Gastritis can be caused by irritation due to excessive alcohol use, chronic vomiting, stress, or the use of certain medications such as aspirin or other anti-inflammatory drugs. It may also be caused by any of the following: Helicobacter pylori (H. pylori), Bile reflux and Infections caused by bacteria and viruses.

Inflammatory cytokines are key hallmark of gastritis. AGS (human gastric cell line) has been extensively used to study cytokine expression under the effect of various test agents. Inhibition of cytokine levels induced by inflammatory agents indicate anti-inflammatory potential in gastric inflammation.

### Study Design

Test System—Human Gastric Adenocarcinoma Cells (AGS)
Number of cells plated—0.1 million cells/well in 24 Well plate
FBS concentrations—0%,
Test items—TF-125 (10.2%), TF-134 (12.4%), TF-136 (13.2%)
Time points—24 h pre-treatment, 24 h stimulation (hu-IL-1β2 ng/ml)
Estimation method—ELISA
Positive Control—Dexamethasone

### Procedure

Cells were plated in 10% FBS in 24-well plates and incubated for 24 hours. Cells were then serum starved in 0% FBS for 24 hours. Cells were then treated with 3 Test items in 0% FBS at various concentrations for 24 h. After incubation, cells were stimulated with inflammatory stimulus (hu-IL-1β2 ng/ml). After 24 hours of stimulation, culture supernatants were collected. Levels of cytokines (IL-8, MIP-1α, RANTES, TNF-α) were determined using ELISA as follows: Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 h at RT. Following a wash (3 times) to remove any unbound conjugate, substrate solution was added to the wells and incubated for 30 minutes at RT in dark. The reaction was stopped by adding Stop solution to each well. The optical density of the color was measured at 450 nm.

### Calculations

Percent Inhibition in each sample was calculated wrt control (hu-IL-1β treated cells).

[(B−A)/B]*100; Where A=Test Item treated cells, B=Control (hu-IL-1β treated) cells.

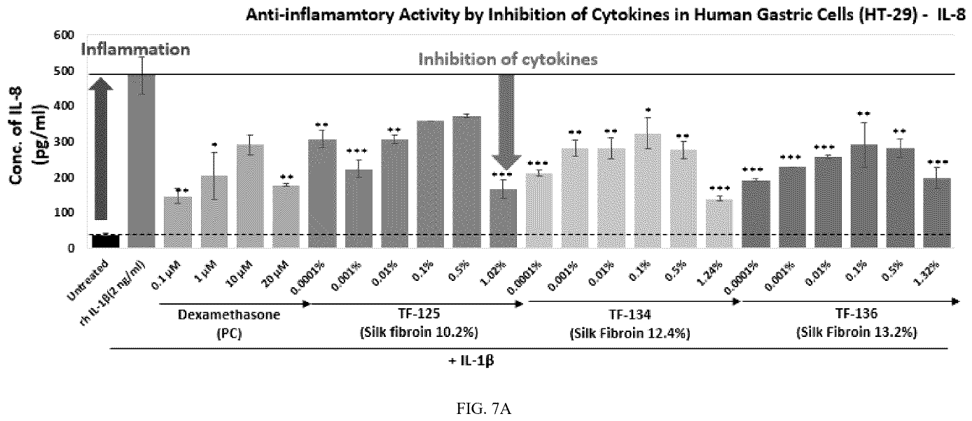

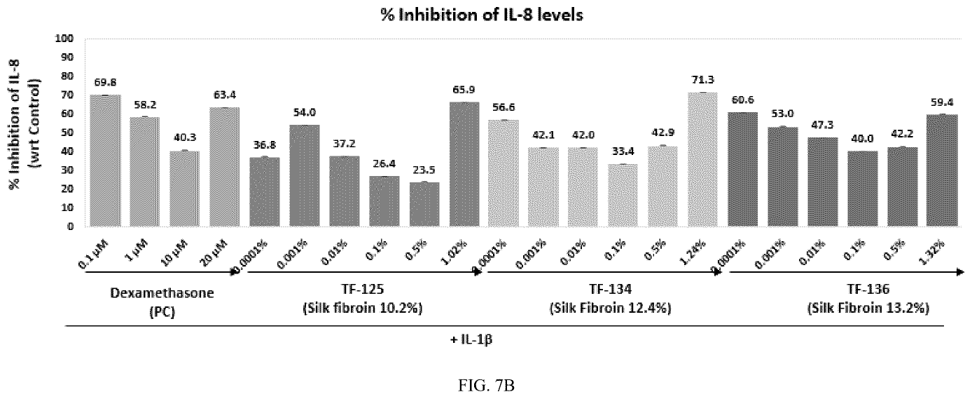

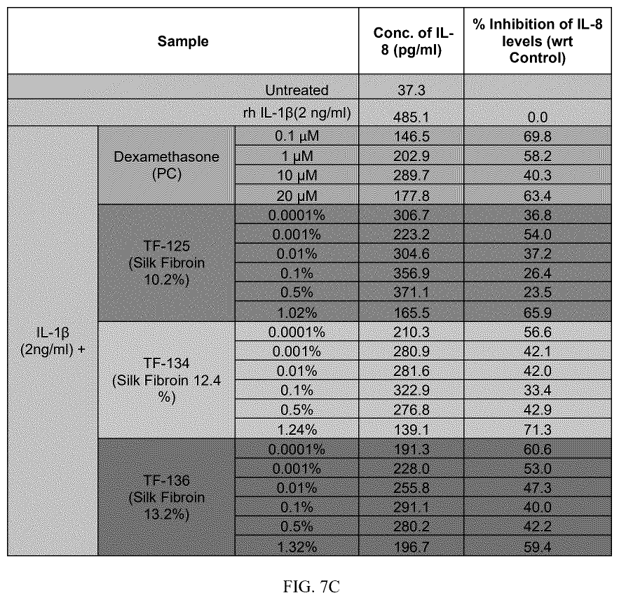

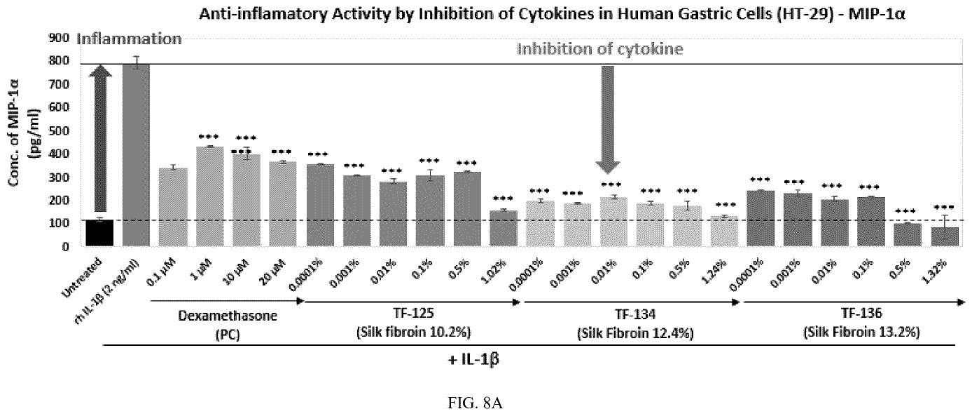

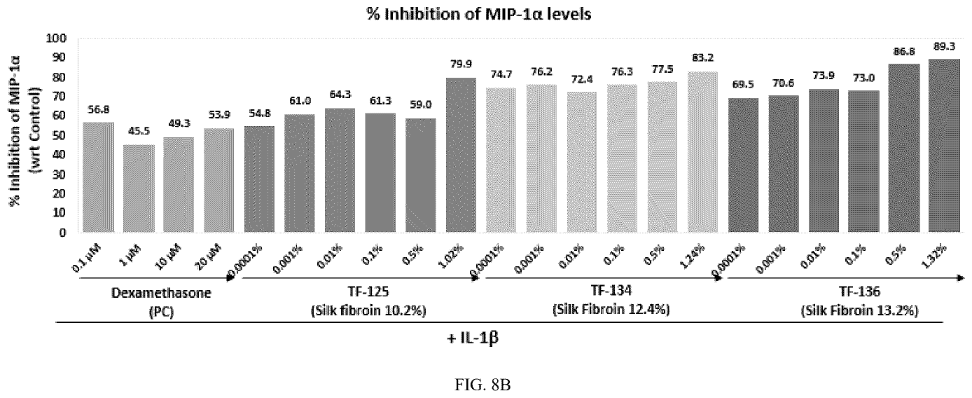

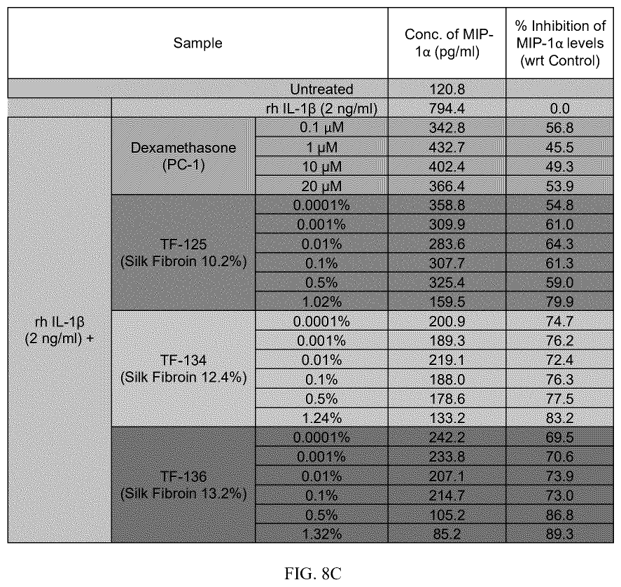

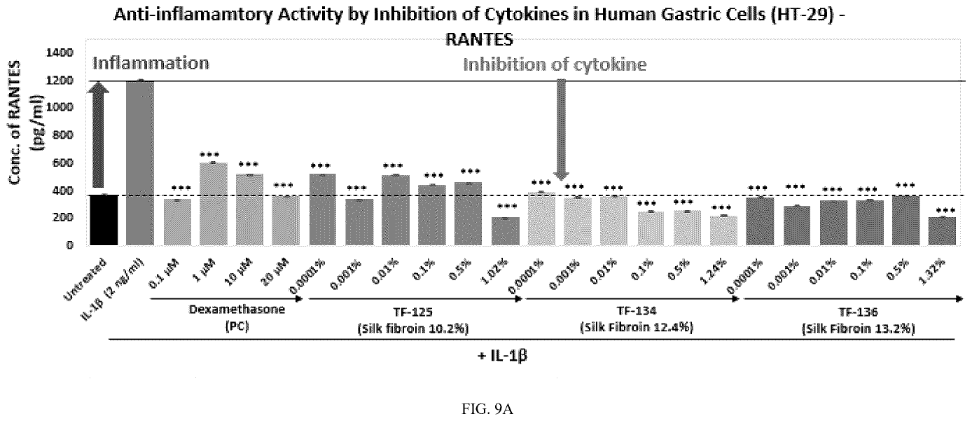

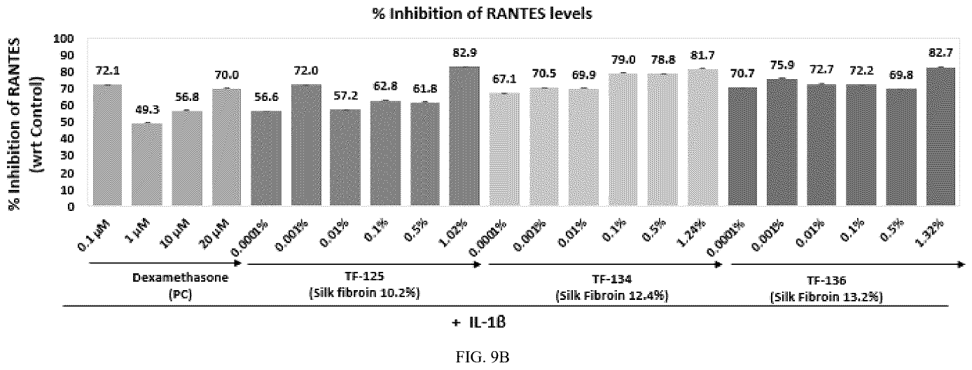

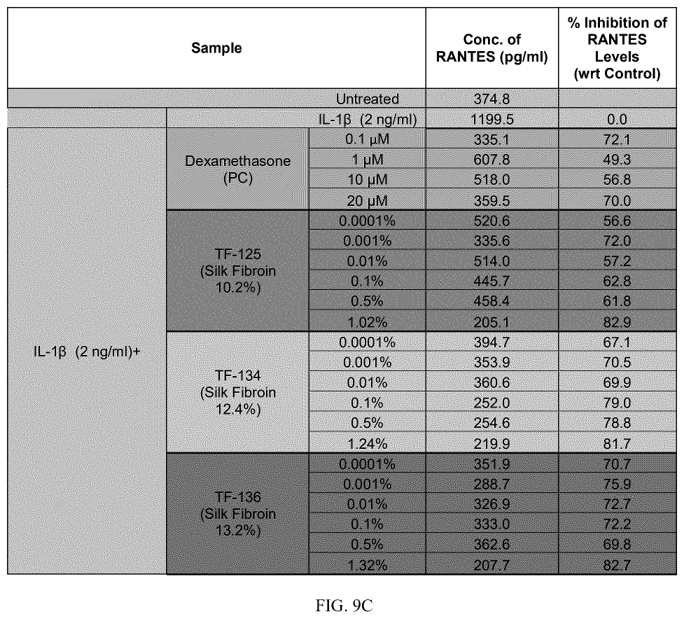

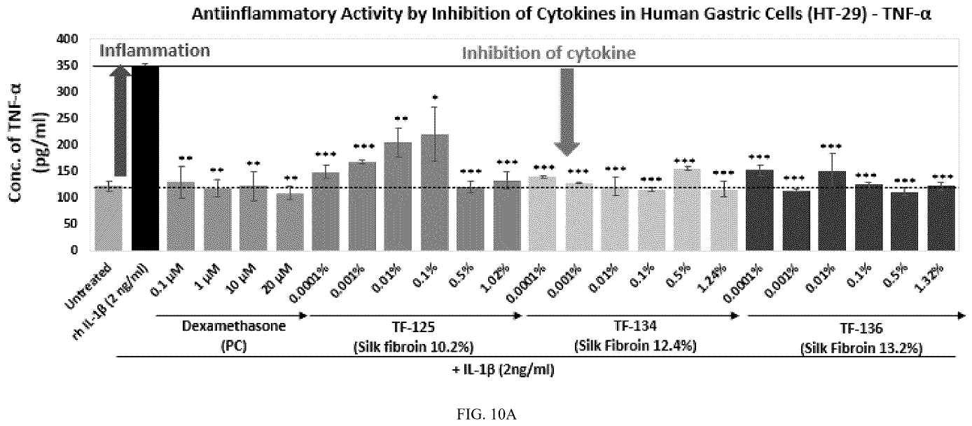

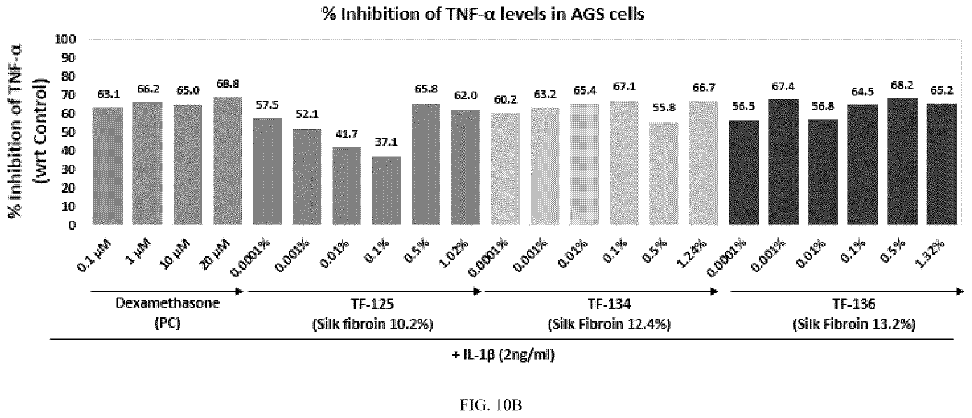

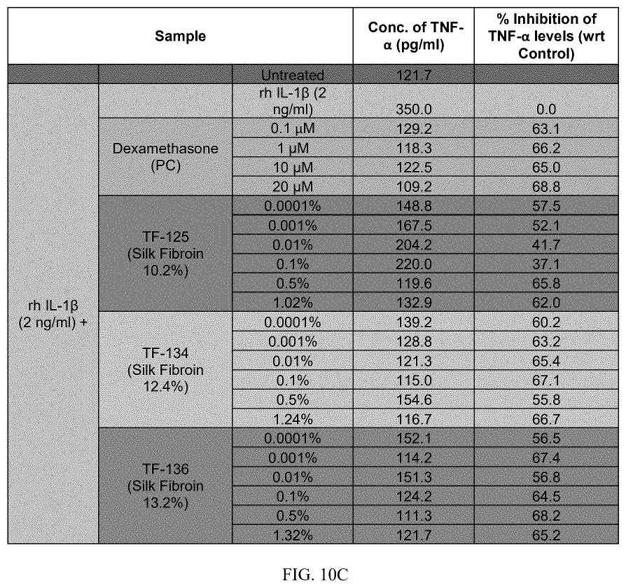

Results: seen at FIGS. 7-10.

Test items comprising silk fibroin ingredient greatly reduced the production of inflammatory cytokines IL-8, MIP-1a, RANTES, and TNF-a as well or better than steroid control (dexamethasone)

Findings: The data shows the anti-inflammatory potential for our silk fibroin peptides. It should translate to a reduction in gastric cell inflammation (oral, esophageal, and stomach) upon direct treatment with the ingredient.

### D. Evaluation of Organ Health Promoting/Body Detoxification Potential of Fibroin Proteins (TF-125, TF-134, TF-136) by Cytoprotection Against Oxidative Stress Using In Vitro Cell Based Assays

Oxygen reactive species (ROS) can play, and in fact they do it, several physiological roles (i.e., cell signaling), and they are normally generated as by-products of oxygen metabolism. When maintained at low or moderate concentrations, free radicals play several beneficial roles for the organism. For example, they are needed to synthesize some cellular structures and to be used by the host defense system to fight pathogens. if in excess, free radicals and oxidants give rise to a phenomenon known as oxidative stress; this is a harmful process that can negatively affect several cellular structures, such as membranes, lipids, proteins, lipoproteins, and deoxyribonucleic acid (DNA).

Oxidative stress is a phenomenon caused by an imbalance between production and accumulation of ROS in cells and tissues and the ability of a biological system to detoxify these reactive products. Superoxide radicals (O2•-), hydrogen peroxide (H2O2), hydroxyl radicals (•OH), and singlet oxygen (1O2) are commonly defined reactive oxygen species (ROS); they are generated as metabolic by-products by biological systems.

Processes, like protein phosphorylation, activation of several transcriptional factors, apoptosis, immunity, and differentiation, are all dependent on a proper ROS production and presence inside cells that need to be kept at a low level, When ROS production increases, they start showing harmful effects on important cellular structures like proteins, lipids, and nucleic acids. A large body of evidence shows that oxidative stress can be responsible, with different degrees of importance, in the onset and/or progression of several diseases (i.e., cancer, diabetes, metabolic disorders, atherosclerosis, and cardiovascular diseases).

Despite this, environmental stressors (i.e., UV, ionizing radiations, pollutants, and heavy metals) and xenobiotics (i.e., antiblastic drugs) contribute to greatly increase ROS production, therefore causing the imbalance that leads to cell and tissue damage (oxidative stress). Excessive production of ROS originated from endogenous and exogenous sources play a dominant role in the initiation and propagation of several diseases. It is therefore an urgent need to explore substances capable of encountering the ROS and resist the damage caused by ROS.

Oxidative stress is an imbalance of free radicals and antioxidants in the body, which can lead to cell and tissue damage. Oxidative stress occurs naturally and plays a role in the aging process. Oxidative stress has more harmful properties than helpful ones. It can break down cell tissue and cause DNA damage. This damage can also result in inflammation. These factors can lead to lifelong diseases like diabetes or cancer, in some cases. Additionally, oxidative stress has been linked to several neurological diseases (i.e., Parkinson's disease, Alzheimer's disease (AD), amyotrophic lateral sclerosis (ALS), multiple sclerosis, depression, and memory loss).

Oxidative stress plays an essential role in the pathogenesis of chronic diseases such as cardiovascular diseases, diabetes, neurodegenerative diseases, and cancer. Long term exposure to increased levels of pro-oxidant factors can cause structural defects at a mitochondrial DNA level, as well as functional alteration of several enzymes and cellular structures leading to aberrations in gene expression. The modern lifestyle associated with processed food, exposure to a wide range of chemicals and lack of exercise plays an important role in oxidative stress induction.

Oxidative stress further plays a crucial role in the development of age-related diseases including arthritis, diabetes, dementia, cancer, atherosclerosis, vascular diseases, obesity, osteoporosis, and metabolic syndromes. ROS are generated within the biological system to modulate the cellular activities such as cell survival, stressor responses, and inflammation.

Elevation of ROS has been associated with the onset and progression of aging. Although ROS generation may not be an essential factor for aging, they are more likely to exacerbate age-related diseases progression via oxidative damage and interaction with mitochondria. Emerging research evidence has suggested that antioxidants can control the autoxidation by interrupting the propagation of free radicals or by inhibiting the formation of free radicals and subsequently reduce oxidative stress, improve immune function, and increase healthy longevity.

Naturally, the organism has several antioxidant defenses to protect against hostile oxidative environments, including classical antioxidant enzymes for example catalase, glutathione peroxidase, and superoxide dismutase as well as non-enzymatic ROS scavengers, such as β-carotene, vitamin C, vitamin E, and uric acid. Antioxidants control the autoxidation by interrupting the propagation of free radicals or by inhibiting the formation of free radicals via different mechanisms. These compounds help in scavenging the species that initiate the peroxidation, breaking the autoxidative chain reaction, quenching O-2, and preventing the formation of peroxides. Antioxidant plays a central role in the termination of oxidative chain reactions by removing the free radical intermediates.

Natural agents such as Polyphenols, flavonoids, carotenoids, minerals, ascorbic acid, Vitamin E, Ubiquinone are well reported antioxidant agents.

### E. Oxidative Stress in Lung

Oxidative stress is the dominant driving force by which the airborne pollutants exert their toxicity in lungs and cause respiratory diseases. Most airborne pollutants are associated with intrinsic oxidative potential and, additionally, stimulate endogenous production of ROS and reactive nitrogen species (RNS).

The lung is persistently exposed to an environment rich in oxygen and thus prone to injury caused by oxidative stress. So as to impede lung tissue damage, lung is endowed with various antioxidant protections such as glutathione, superoxide dismutase, β-carotene, vitamins C and E, uric acid and heme-oxygenase.

However sometime reactive oxygen or nitrogen species defeat biological antioxidant protection, and an oxidative stress leads to a number of lung disorders. Recently, several scientific reports have verified the assumption of oxidative injury in cystic fibrosis, asthma, COPD and a direct injure to epithelial cells by reactive oxygen species.

Existing therapy is used to control abnormal mucus secretion and inflammation in order to delay lung tissue damage. There is compelling evidence from preclinical and clinical studies that pulmonary antioxidants play a pivotal role in protecting from environmental pulmonary diseases.

Oxygen provides this vast potential energy source, but the same chemical reactivity which provides this potential also can have detrimental effects. The lung evolved as an organ that can efficiently promote gas exchange for the entire organism but as such, the lung is highly susceptible to its external environment. Oxygen can be transformed through both enzymatic and non-enzymatic processes into ROS and RNS, which can lead to protein, lipid, and DNA damage. Under normal conditions ROS/RNS concentrations are minimized through the activity of antioxidants located both intracellularly and in the epithelial lining fluid of the lung.

Oxidative stress in the lung results when the antioxidant capacity is overwhelmed or depleted through external exposures, such as altered oxygen tension or air pollution, or internally. Internal sources of oxidative stress include systemic disease and the activation of resident cells and inflammatory cells recruited in response to an exposure or systemic response. Pulmonary responses to oxidative stress include activation of oxidases, lipid peroxidation, increases in nitric oxide, and autophagy. These internal and external exposures with the subsequent pulmonary responses contribute to development of diseases directly linked to oxidative stress. These include asthma, COPD, and lung cancers. While the vulnerability of the lung to oxidative stress is acknowledged, few effective preventative strategies or therapeutics are currently available.

Human lung epithelial cell line A549 has been widely reported as a model system to study and investigate the protective effect of various agents against oxidative stress induced damage. Hydrogen peroxide or t-butyl hydro peroxide cause oxidative damage to cells and key markers such as Cyto-protection (restoration of cell viability damaged by t-BHP), anti-apoptotic effect by JC-1 and cell cycle, ROS are reported as end points

1a)—Cytoprotective effect against oxidative stress in lung cells (A549) by restoration of cell viability

### Study Design

Test System—Human lung alveolar epithelium cell line (A549)
Number of cells plated—10000 cells/well plate
FBS concentrations—0.1%
Test items—TF-125(10.2%), TF-134(12.4%), TF-136 (13.2%)
Time points—48 h pretreatment, t-BHP (1 mM) damage
Estimation method—MTT assay
Positive Control—Quercetin

### Procedure

Cells were plated in 96-well plates in 10% FBS and incubated for 24 hours. Cells were then pre-treated with Test Items at various concentrations for 48 hours. Cells were exposed to oxidative damage t-BHP (1 mM) for 3.5 hours. After 3.5 hours, cell viability was assessed by MTT assay, 20 μl of MTT solution was added to each well and cells were incubated for 3 hours at 37° C. After incubation, supernatants were removed and 150 μl DMSO added to all wells to extract formazan crystals. A purple-colored formazan complex was formed. Absorbance of each well was measured at 540 nm.

### Calculation

Protective effect on cell viability was calculated wrt controls (t-BHP damaged cells). {(Absorbance of TI+t-BHP)−(Absorbance of t-BHP alone)/(Absorbance of Untreated)−(Absorbance of t-BHP alone)}*100

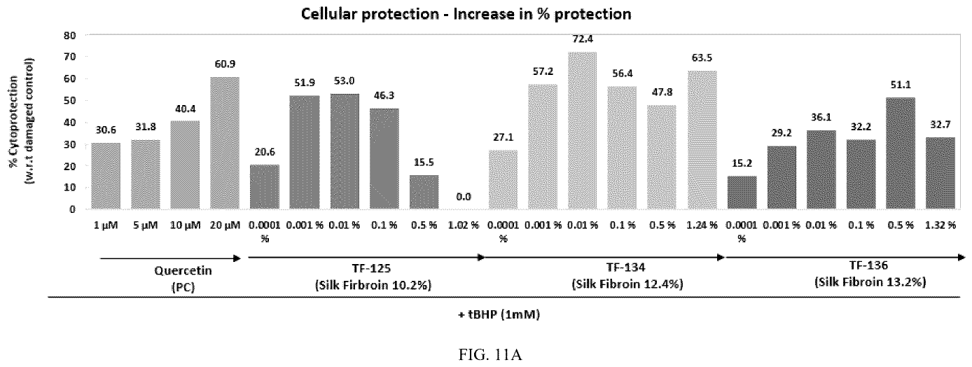

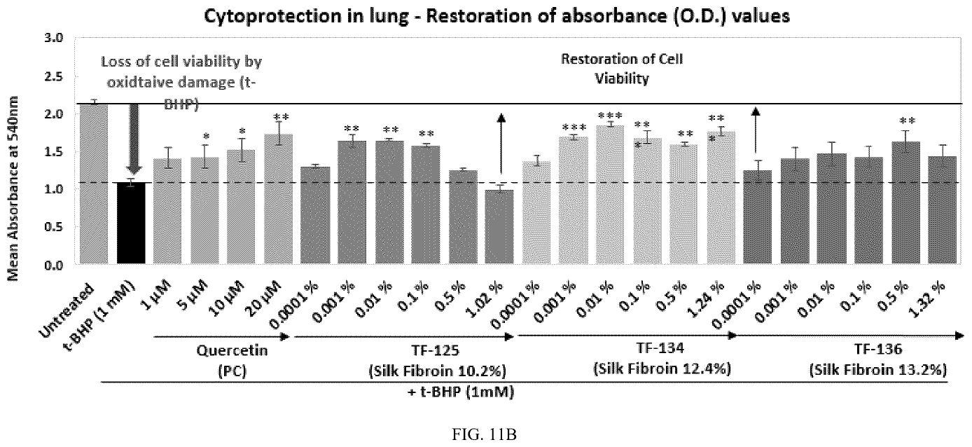

Results: seen at FIG. 11.

1b)—Cytoprotective Effect Against Oxidative Stress in Lung Cells (A549) by Antiapoptotic Effect (JC-1)

### Study Design

Test System—Human lung alveolar epithelium cell line (A549)
Number of cells plated—10,000
FBS concentrations—0.1% FBS
Test items—TF-125(10.2%), TF-134(12.4%), TF-136 (13.2%)
Time point—48 h pretreatment, t-BHP (1 mM)
End points—Mitochondrial Membrane Potential using JC-1 dye.
Positive Control—Quercetin

### Procedure

Cells were plated in 10% FBS in 96-well plates and incubated for 24 hours. Cells were then treated with 3 Test items in 0.1% FBS at various concentrations for 24 hours. After 48 hours, damage was induced using t-BHP (1 mM) damage in cells. After 3.5 hours, the effect on Mitochondrial Membrane Potential (MMP) was determined by JC-1 assay. Active mitochondria in live cells exhibit brighter red fluorescence signals compared to mitochondria with lower membrane potential in apoptotic cells which fluoresce green with JC-1 dye. Ratio of Red: Green indicates degree of MMP. Inhibition of MMP shows depolarization of MMP. After incubation, medium from each well was removed. 100 μl of 10 μM JC-1 dye in PBS was added to each well and cells were incubated at 37° C. for 25 minutes. After incubation, cells were rinsed with PBS to remove dye and Fluorescence was measured at 528/590 for red and 485/528 for green. Ratio of Red: green (healthy cells) was calculated.

### Calculations

Increase of Mitochondrial membrane potential was calculated wrt t-BHP damage control. [(A−B)/B]*100; Where A=Ratio of Red: Green in Test Item treated cells, B=Ratio of Red Green in t-BHP damage control cells

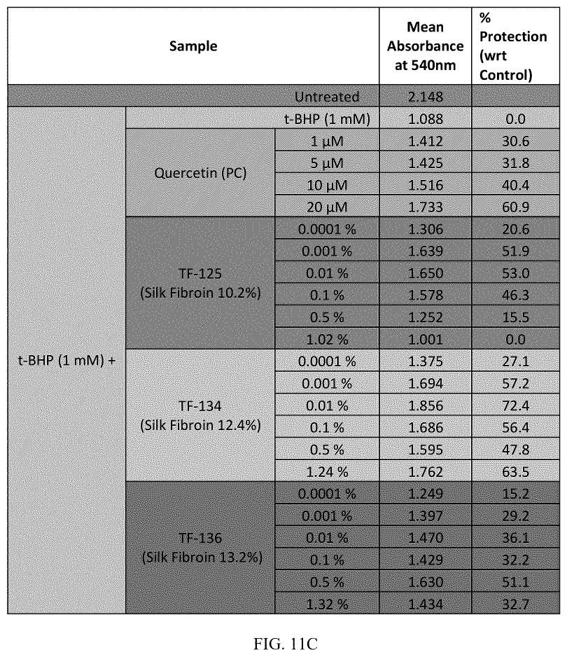

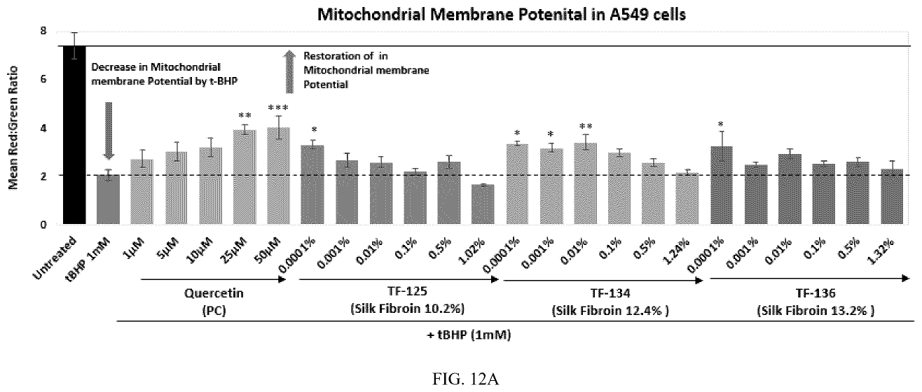

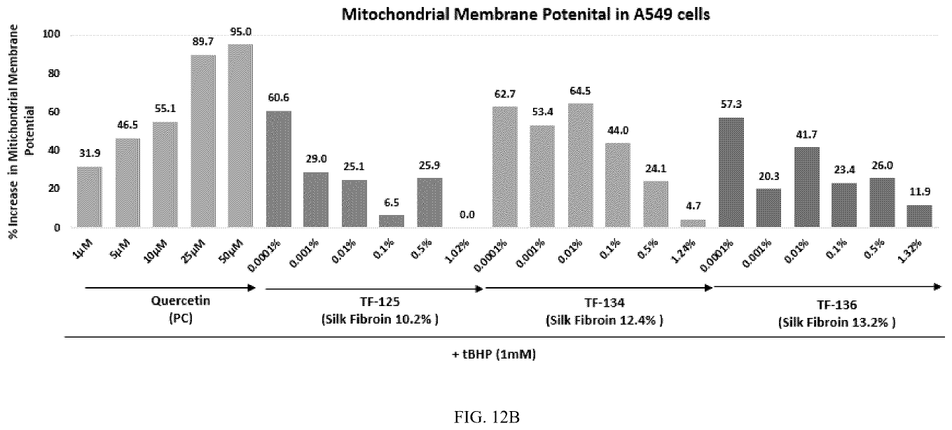

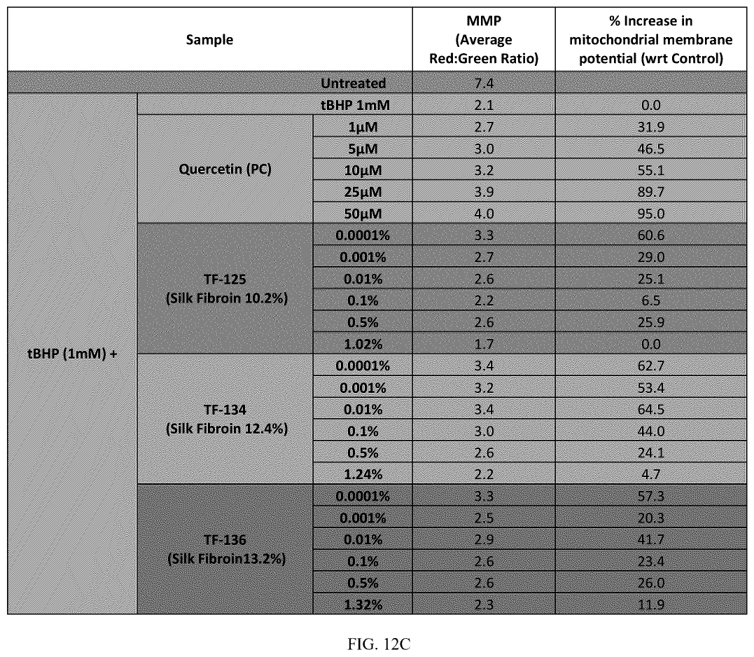

Results: seen at FIG. 12.

1c)—Cytoprotective Effect Against Oxidative Stress in Lung Cells (A549) by Antiapoptotic Effect (Cell Cycle)

### Study Design

Test System—Human lung alveolar epithelium cell line (A549)
Number of cells plated—0.1 million cells/12-well plates
FBS concentrations—0.5%,
Test items—TF-125(10.2%), TF-134(12.4%), TF-136 (13.2%)
Time points—48 h pretreatment, t-BHP (1 mM)
Estimation method—SubG0/G1 apoptotic cells by Propidium iodide solution
Positive Control—Quercetin

### Procedure

Cells were plated in 10% FBS in 12-well plates and incubated for 24 hours. Cells were then Serum starved with 0.5% FBS for 24 hours. After 24 hours, cells were treated with 3 Test items in 0.5% FBS at various concentrations. After 48 hours, damage was induced using t-BHP (1 mM) damage in cells. After 3.5 hours, the anti-apoptotic effect was measured by Cell-cycle analysis. After incubation, medium from each well was removed and cells were harvested by trypsinization and centrifuged at 450 g for 5 minutes (low brake). Supernatant was discarded and the cell pellet was washed with 1× PBS at 450 g for 5 min (low brake). Cells were fixed with Ice-cold 70% ethanol (500 μl) and stored at 4° C. for 24 hours prior to staining. For staining, Ethanol fixed cells were centrifuged at low brake and washed with PBS. 200 μl of cell cycle reagent was added to each sample at kept at RT for staining for 30 min in dark. After incubation, cells were acquired using flow cytometer.

### Calculations

Decrease in apoptotic cell population (sub G0/G1) was calculated wrt t-BHP damage control. [(B−A)/B]*100; Where A=% subG0/G1 cells in Test Item treated, B=% subG0/G1 cells in t-BHP damage Control

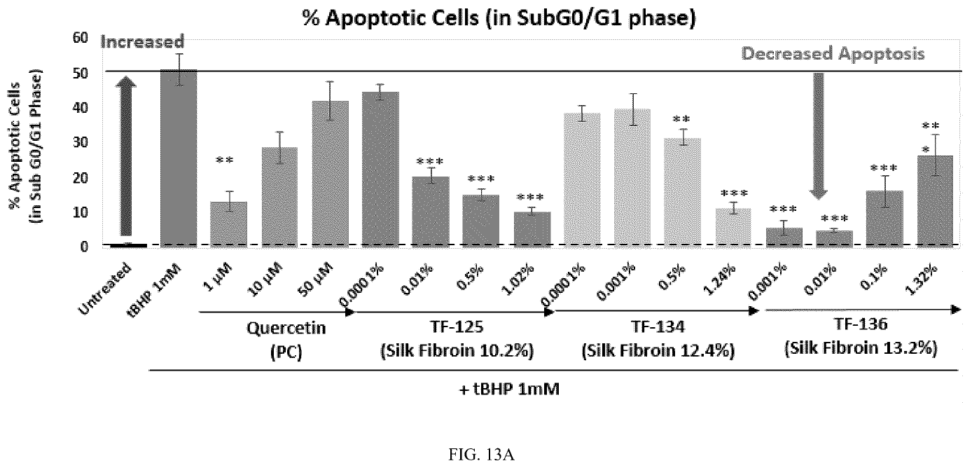

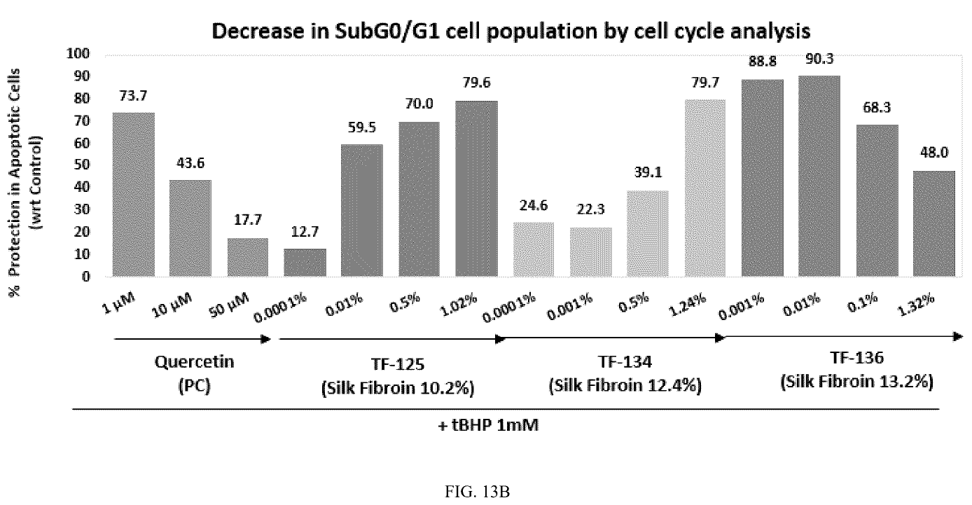

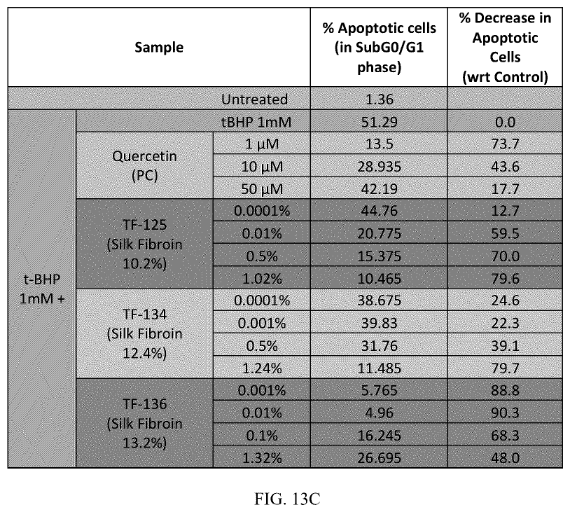

Results: seen at FIG. 13.

Findings: This above data shows that the silk fibroin peptides according to the disclosure improve cell viability and reduce cell death in the presence of oxidative stress/damage leading to a cytoprotective effect suggesting a protective effect with direct treatment to the lung cells.

### F. Liver Protection Cytoprotective Effect Against Oxidative Stress

### a) Restoration of Cell Viability

### b) Anti-Apoptotic Effect

Redox state constitutes an important background of numerous liver disorders. The redox state participates in the course of inflammatory, metabolic and proliferative liver diseases. ROS are primarily produced in the mitochondria and in the endoplasmic reticulum of hepatocytes via the cytochrome P450 enzymes. Under the proper conditions, cells are equipped with special molecular strategies that control the level of oxidative stress and maintain a balance between oxidant and antioxidant particles.

Oxidative stress represents an imbalance between oxidant and antioxidant agents. Liver is a major organ attacked by ROS. Parenchymal cells are primary cells subjected to oxidative stress induced injury in the liver. Hepatocytic proteins, lipids and DNA are among the cellular structures that are primarily affected by ROS and reactive nitrogen species. The process results in structural and functional abnormalities in the liver.

Oxidative stress has been considered as a conjoint pathological mechanism, and it contributes to initiation and progression of liver injury. A lot of risk factors, including alcohol, drugs, environmental pollutants and irradiation, may induce oxidative stress in liver, which in turn results in severe liver diseases, such as alcoholic liver disease and non-alcoholic steatohepatitis.

Application of antioxidants signifies a rational curative strategy to prevent and cure liver diseases involving oxidative stress. Multiple agents are reported to exert beneficial effects in hepatic disorders induced by oxidative stress. Oxygen is vital for life as it is required for many different enzymatic reactions involved in intermediate metabolism and xenobiotic biotransformation. Oxygen consumption in the electron transport chain of mitochondria is used to drive the synthesis of ATP to meet the energetic demands of cells. A complex antioxidant system has been developed in mammals to relieve oxidative stress. However, excessive reactive species derived from oxygen and nitrogen may still lead to oxidative damage to tissue and organs. Oxidative stress has been considered as a conjoint pathological mechanism, and it contributes to initiation and progression of liver injury. The cellular impairment induced by oxidative stress in the liver is related with mitochondrial dysfunction as well as with the depletion of antioxidant proteins such as glutathione or superoxide dismutase. Moreover, the cellular redox imbalance produced in liver diseases induces the activation of apoptotic and inflammation signaling pathways enhancing cell death.

Hepatocytes are well equipped with non-enzymatic and enzymatic antioxidant defense systems that neutralize free radicals. Liver is a major organ attacked by ROS. Parenchymal cells are primary cells subjected to oxidative stress induced injury in the liver.

Human liver cell line (HepG2 cells) is widely reported as model system to investigate the protective effect of Test agents against oxidative stress induced stress. Hydrogen peroxide (H2O2) or t-butyl hydro peroxide (t-BHP) cause oxidative damage to cells and key markers such as Cytoprotection (restoration of cell viability damaged by t-BHP), anti-apoptic effect by JC-1 and cell cycle, ROS are reported as end points.

2a)—Cytoprotective Effect Against Oxidative Stress in Liver Cells (HepG2) by Restoration of Cell Viability

### Study Design

Test System—Human Hepatocellular carcinoma cell line (HepG2)
Number of cells plated—10000 cells/well plate
FBS concentrations—0.1%
Test items—TF-125(10.2%), TF-134(12.4%), TF-136 (13.2%)
Time points—48 h pretreatment, t-BHP (250 μM) damage
Estimation method—MTT assay
Positive Control—Silymarin, Resveratrol

### Procedure

Cells were plated in 96-well plates in 10% FBS and incubated for 24 hours. Cells were then pre-treated with Test Items at various concentrations for 48 hours. Cells were exposed to oxidative damage t-BHP (250 μM) for 3.5 hours. After 3.5 hours, cell viability was assessed by MTT assay, 20 μl of MTT solution was added to each well and cells were incubated for 3 h at 37° C. After incubation, supernatants were removed and 150p DMSO was added to all wells to extract formazan crystals. A purple-colored formazan complex was formed. Absorbance of each well was measured at 540 nm.

### Calculation

Protective effect on cell viability was calculated wrt controls (t-BHP damaged cells). {(Absorbance of TI+t-BHP)−(Absorbance of t-BHP alone)/(Absorbance of Untreated)−(Absorbance of t-BHP alone)}*100

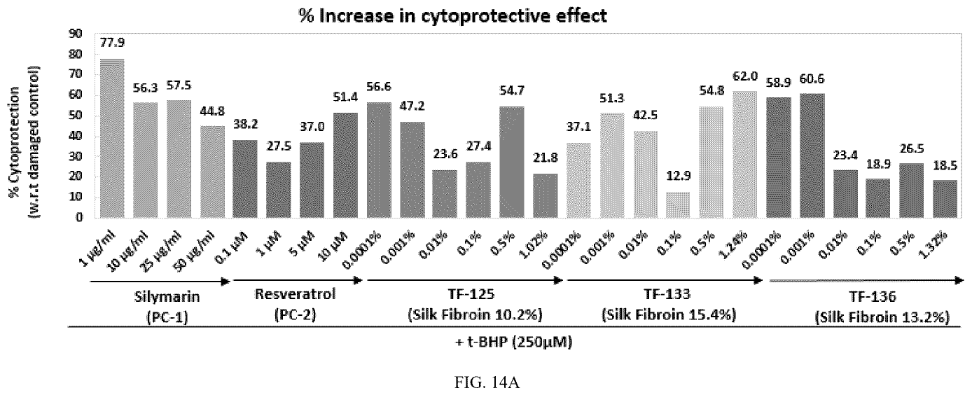

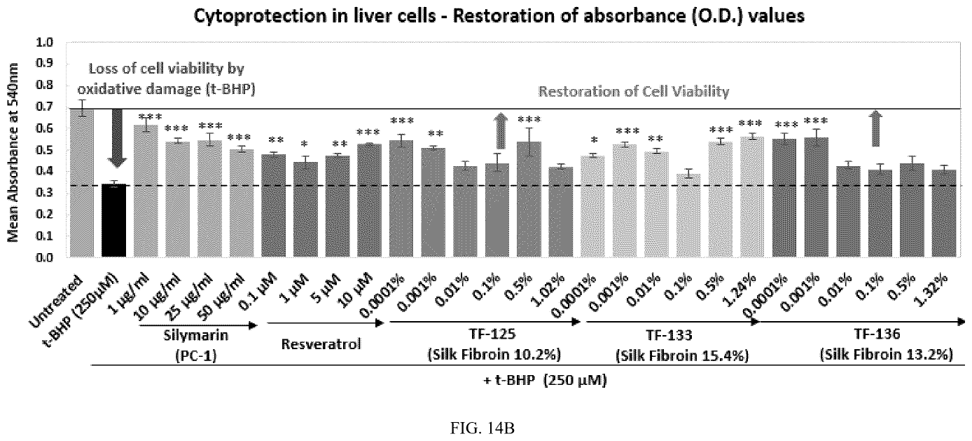

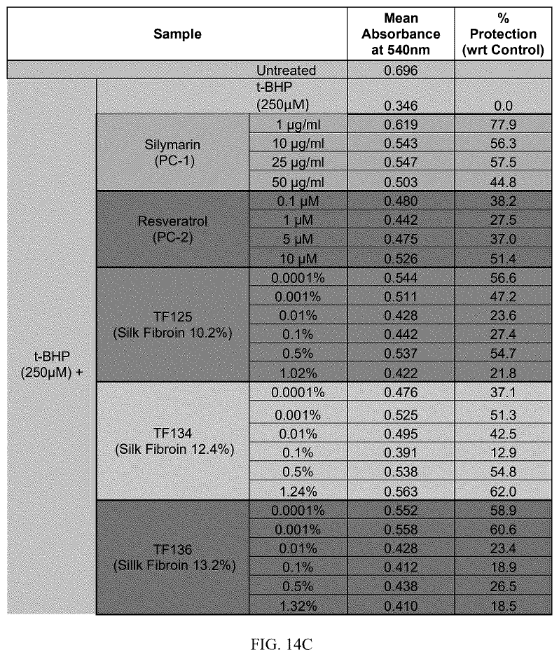

Results: seen at FIG. 14.

2b)—Cytoprotective Effect Against Oxidative Stress in Liver Cells (HepG2) by Antiapoptotic Effect (JC-1)

### Study Design

Test System—Human Hepatocellular carcinoma cell line (HepG2)
Number of cells plated—10,000
FBS concentrations—0.1% FBS
Test items—TF-125(10.2%), TF-134(12.4%), TF-136 (13.2%)
Time point—48 h pretreatment, t-BHP (250 μM) damage
End points—Mitochondrial Membrane Potential using JC-1 dye.
Positive Control—Silymarin, Resveratrol

### Procedure

Cells were plated in 10% FBS in 96-well plates and incubated for 24 hours. Cells were then treated with 3 Test items in 0.1% FBS at various concentrations for 24 hours. After 48 hours, damage was induced using t-BHP (250 μM) damage in cells. After 3.5 hours, the effect on Mitochondrial Membrane Potential (MMP) was determined by JC-1 assay. Active mitochondria in live cells exhibit brighter red fluorescence signals compared to mitochondria with lower membrane potential in apoptotic cells which fluoresce green with JC-1 dye. Ratio of Red: Green indicates degree of MMP. Inhibition of MMP shows depolarization of MMP. After incubation, medium from each well was removed. 100 μl of 10 μM JC-1 dye in PBS was added to each well and cells were incubated at 37° C. for 25 minutes. After incubation, cells were rinsed with PBS to remove the dye and Fluorescence was measured at 528/590 for red and 485/528 for green. Ratio of Red: green (healthy cells) was calculated.

### Calculations

Increase of Mitochondrial membrane potential was calculated wrt t-BHP damage control. [(A−B)/B]*100; B)/B]*100; Where A=Ratio of Red: Green in Test Item treated cells, B=Ratio of Red: Green in t-BHP damage control cells.

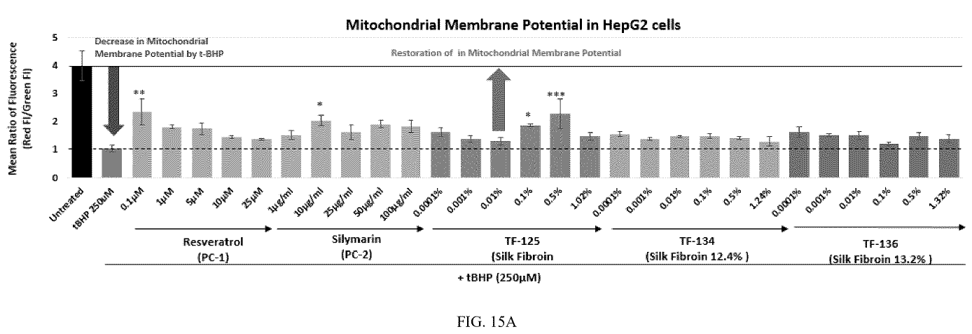

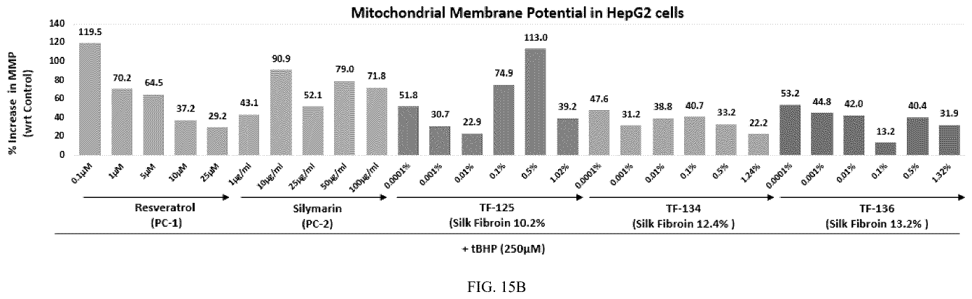

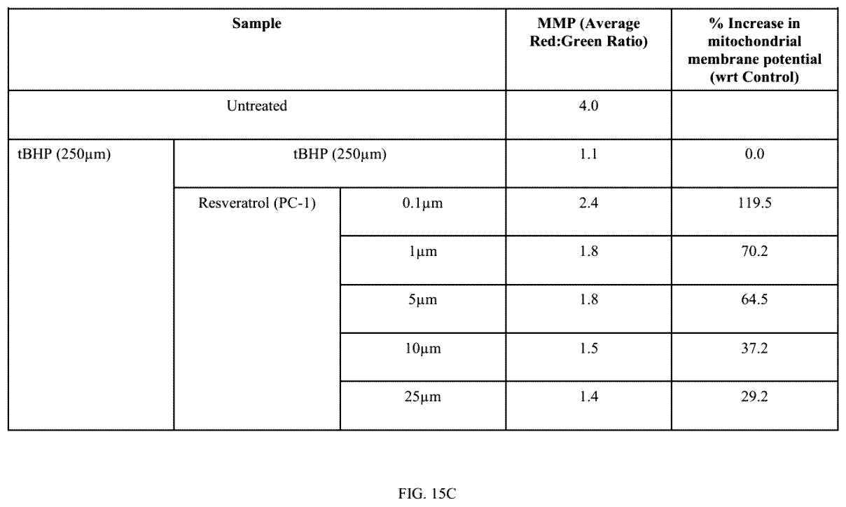

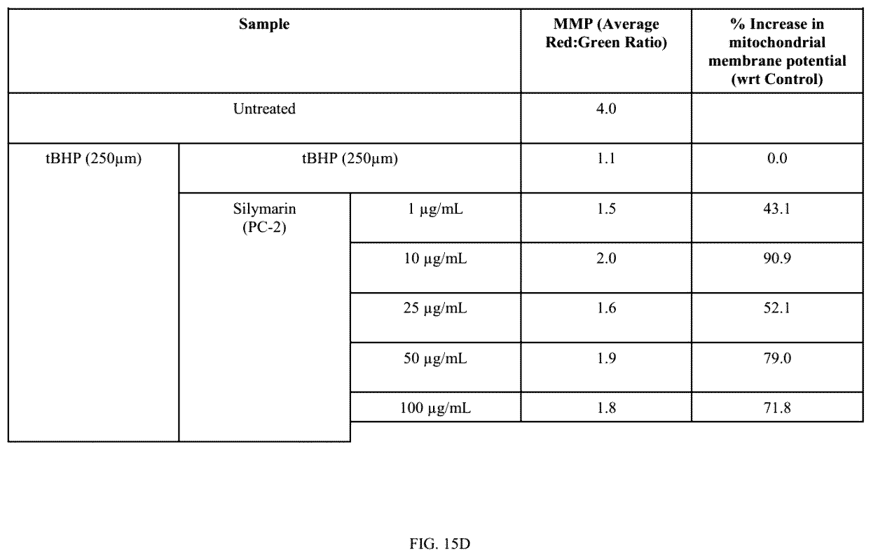

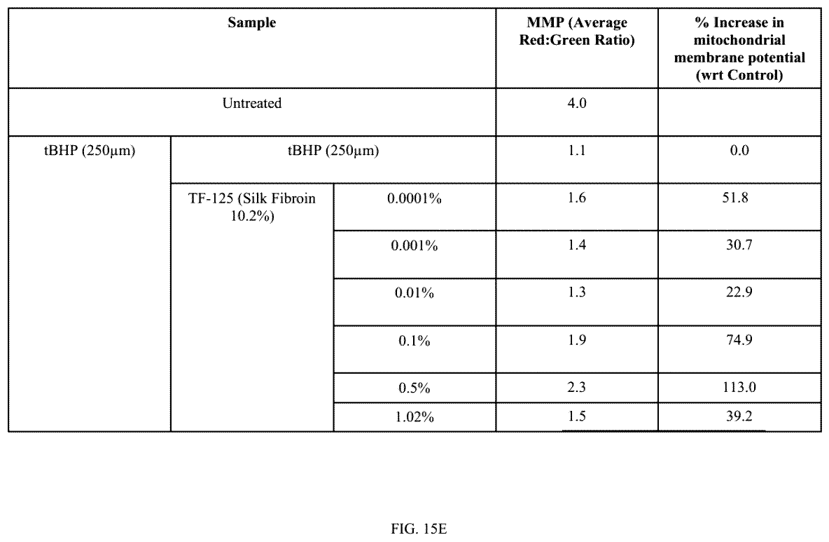

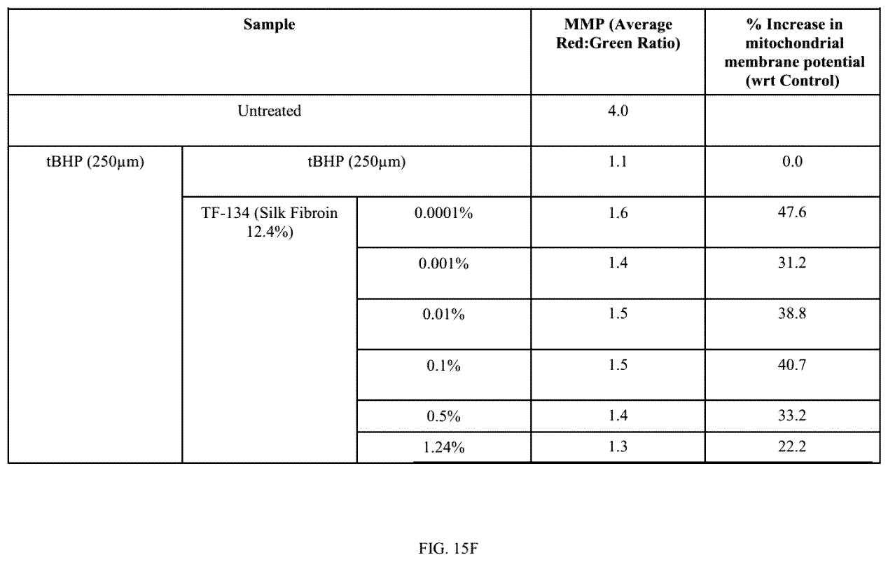

Results: seen at FIG. 15.

2b)—Cytoprotective Effect Against Oxidative Stress in Liver Cells (HepG2) by Antiapoptotic Effect

### Study Design

Test System—Human Hepatocellular carcinoma cell line (HepG2)
Number of cells plated—0.1 million cells/12-well plates
FBS concentrations—0.5%,
Test items—TF-125(10.2%), TF-134(12.4%), TF-136 (13.2%)
Time points—48 h pre-treatment, t-BHP (250 μM) damage
Estimation method—SubG0/G1 apoptotic cells by Propidium iodide solution
Positive Control—Silymarin, Resveratrol

### Procedure

Cells were plated in 10% FBS in 12-well plates and incubated for 24 hours. Cells were then Serum starved with 0.5% FBS for 24 hours. After 24 hours, cells were treated with 3 Test items in 0.5% FBS at various concentrations. After 48 hours, damage was induced using t-BHP (250 μM) damage in cells. After 3.5 hours, the anti-apoptotic effect was measured by Cell-cycle analysis. After incubation, medium from each well was removed and cells were harvested by trypsinization and centrifuged at 450 g for 5 minutes (low brake). Supernatant was discarded and cell pellet was washed with 1× PBS at 450 g for 5 minutes (low brake). Cells were fixed with Ice-cold 70% ethanol (500 μl) and stored at 4° C. for 24 hours prior to staining. For staining, Ethanol fixed cells were centrifuged at low brake and washed with PBS. 200 μl of cell cycle reagent was added to each sample at kept at RT for staining for 30 min in dark. After incubation, cells were acquired using flow cytometer.

### Calculations

Decrease in apoptotic cell population (sub G0/G1) was calculated wrt t-BHP damage control. [(B−A)/B]*100; Where A=% subG0/G1 cells in Test Item treated B=% subG0/G1 cells in t-BHP damage Control.

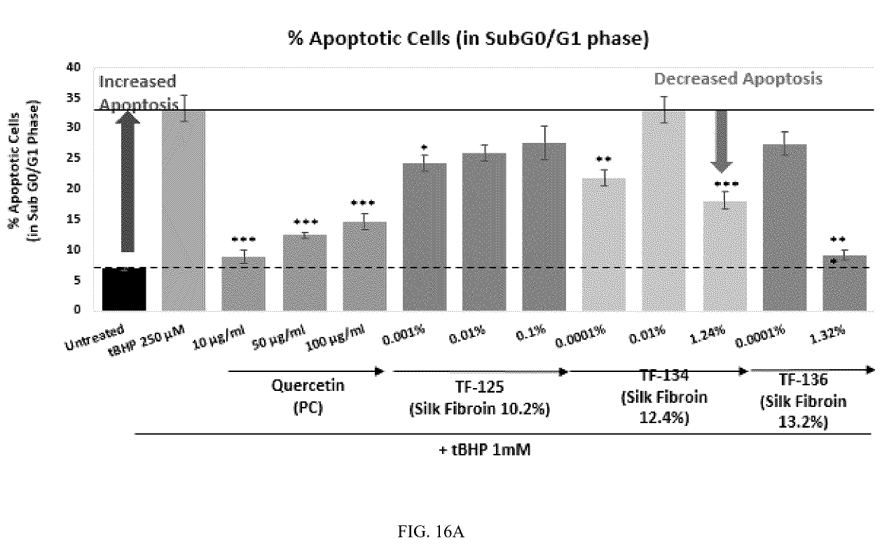

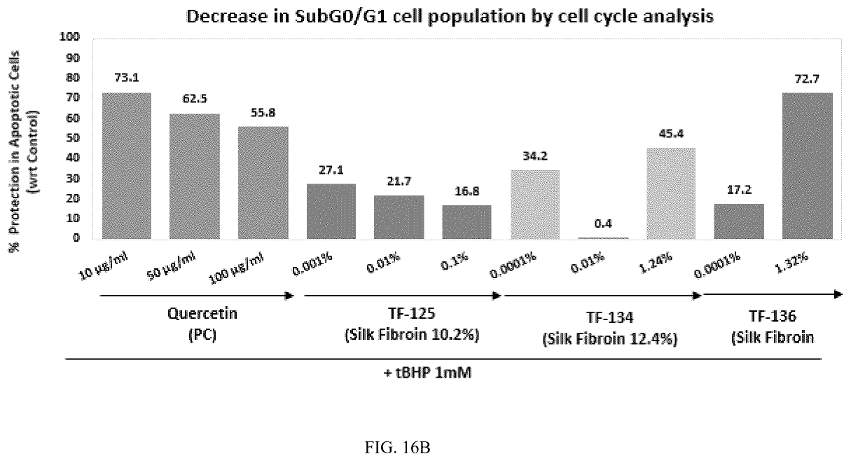

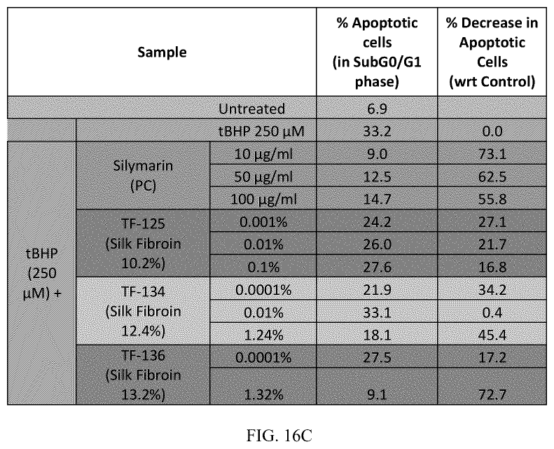

Results: seen at FIG. 16.

Findings: The above data shows that in the presence of oxidative stress/damage the silk fibroin peptides according to the disclosures improve liver cell viability and reduce liver cell death leading to a cytoprotective effect suggesting a protective effect with treatment to the liver cells.

### G. Gastric Protection/Cytoprotective Effect Against Oxidative Stress

### a) Restoration of Cell Viability

### b) Anti-Apoptotic Effect

Reactive oxygen species (ROS) are generated as by-products of normal cellular metabolic activities. Superoxide dismutase, glutathione peroxidase, and catalase are the enzymes involved in protecting cells from the damaging effects of ROS. ROS are produced in response to ultraviolet radiation, cigarette smoking, alcohol, nonsteroidal anti-inflammatory drugs, ischemia-reperfusion injury, chronic infections, and inflammatory disorders.

The gastrointestinal (GI) tract is a key source of ROS. Despite the protective barrier provided by the epithelial layer, ingested materials and pathogens can cause inflammation by activating the epithelium, polymorphonuclear neutrophils (PMNs), and macrophages to produce inflammatory cytokines and other mediators that contribute further to oxidative stress. Various GI pathological conditions including gastroduodenal ulcers, GI malignancies, and inflammatory bowel disease (IBD) arise in part from oxidative stress.

The stomach is a sensitive digestive organ that is susceptible and exposed to exogenous pathogens from the diet. In response to such pathogens, the stomach induces oxidative stress, which might be related to the development of gastric organic disorders such as gastritis, gastric ulcers, and gastric cancer, as well as functional disorders such as functional dyspepsia. In particular, the bacterium Helicobacter pylori plays a major role in eliciting and confronting oxidative stress in the stomach.

Molecular oxygen (O2) is not only essential for the survival of aerobic organisms, its reduction to H2O via mitochondrial respiration complexes provides ATP, but paradoxically contributes to cell death. Partially reduced O2, collectively named ROS, are highly reactive and continuously produced as by-products of cellular respiration. OS are also generated during enzymatic reactions. ROS include radical compounds such as superoxide (O2•-), hydroxyl radicals (HO•), lipid hydroperoxides, and reactive nonradical compounds including singlet oxygen (1O2), hydrogen peroxide (H2O2), hypochlorous acid (HOCl).

Oxidative stress also contributes to various GI diseases including gastroduodenal ulcers, inflammatory bowel disease and GI malignancies such as gastric and colorectal cancer [30]. The gastrointestinal (GI) tract is a key source of ROS. Despite the protective barrier provided by the epithelial layer, ingested materials and pathogens can cause inflammation by activating the epithelium, polymorphonuclear neutrophils (PMNs), and macrophages to produce inflammatory cytokines and other mediators that contribute further to oxidative stress. Various GI pathological conditions including gastroduodenal ulcers, GI malignancies, and inflammatory bowel disease (IBD) arise in part from oxidative stress.

Human gastric cell line (AGS) is very well reported by various researchers for understanding the protective role of test agents in gastric system against oxidative stress induced damage. Hydrogen peroxide (H2O2) or t-butyl hydro peroxide (t-BHP) have been oxidative damage to cells and key markers such as Cytoprotection (restoration of cell viability damaged by t-BHP), anti-apoptic effect by JC-1 and cell cycle, ROS are reported as end points.

3a)—Cytoprotective Effect Against Oxidative Stress in Gastric Cells (AGS) by Restoration of Cell Viability

### Study Design

Test System—Human adenocarcinoma gastric cell line (AGS)
Number of cells plated—10000 cells/well plate
FBS concentrations—0.1%
Test items—TF-125(10.2%), TF-134(12.4%), TF-136 (13.2%)
Time points—48 h pretreatment, t-BHP (1 mM) damage
Estimation method—MTT assay
Positive Control—Carnosic Acid

### Procedure

Cells were plated in 96-well plates in 10% FBS and incubated for 24 hours. Cells were then pre-treated with Test Items at various concentrations for 48 hours. Cells were exposed to oxidative damage t-BHP (1 mM) for 3.5 hours and cell viability was assessed by MTT assay, 20 μl of MTT solution was added to each well and cells were incubated for 3 hours at 37° C. After incubation, supernatants were removed and 150 μl DMSO added to all wells to extract formazan crystals. A purple-colored formazan complex was formed. Absorbance of each well was measured at 540 nm.

### Calculation

Protective effect on cell viability was calculated wrt controls (t-BHP damaged cells). (Absorbance of TI+t-BHP)−(Absorbance of t-BHP alone)/(Absorbance of Untreated)−(Absorbance of t-BHP alone)}*100

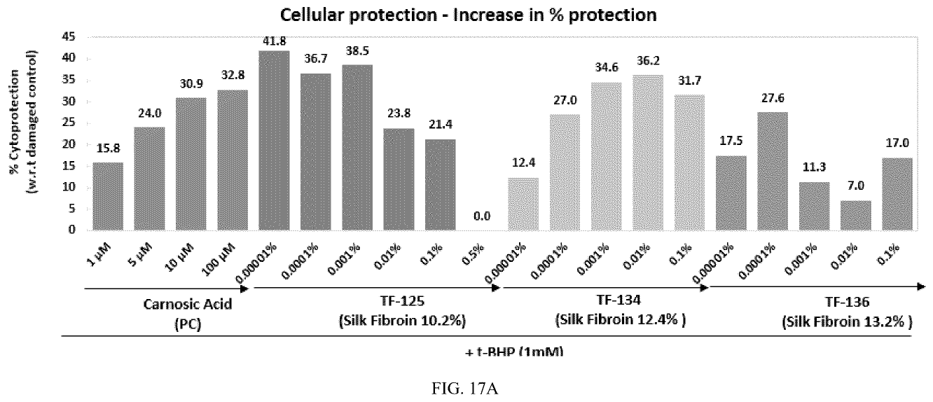

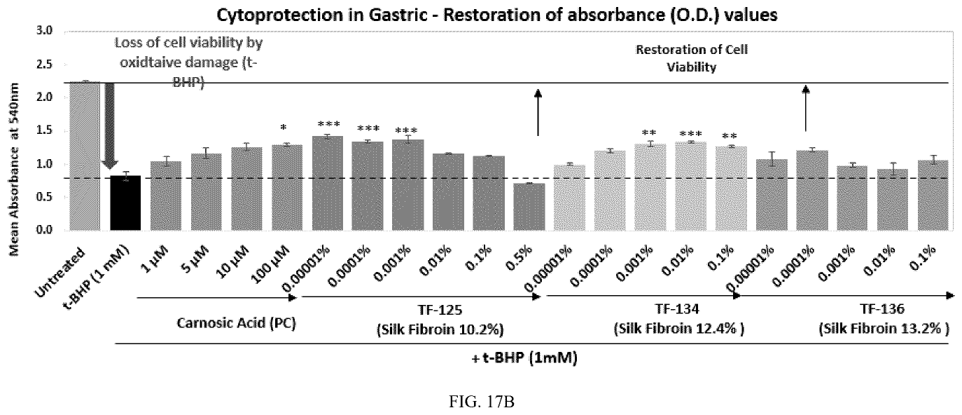

Results: seen at FIG. 17.

3b)—Cytoprotective Effect Against Oxidative Stress in Gastric Cells (AGS) by Antiapoptotic Effect (JC-1)

### Study Design

Test System—Human adenocarcinoma gastric cell line (AGS)
Number of cells plated—10,000 cells/well
FBS concentrations—0.1% FBS
Test items—TF-125(10.2%), TF-134(12.4%), TF-136 (13.2%)
Time point—48 h pretreatment, t-BHP (1 mM)
End points—Mitochondrial Membrane Potential using JC-1 dye.
Positive Control—Carnosic Acid

### Procedure

Cells were plated in 10% FBS in 96-well plates and incubated for 24 hours. Cells were then treated with 3 Test items in 0.1% FBS at various concentrations for 24 hours. After 48 hours, damage was induced using t-BH1P (1 mM) damage in cells. After 3.5 hours, the effect on Mitochondrial membrane Potential was determined by JC-1 assay. Active mitochondria in live cells exhibit brighter red fluorescence signals compared to mitochondria with lower membrane potential in apoptotic cells which fluoresce green with JC-1 dye. Ratio of Red: Green indicates degree of MMP. Inhibition of MMP shows depolarization of MMP. After incubation, medium from each well was removed. 100 μl of 10 μM JC-1 dye in PBS was added to each well and cells were incubated at 37° C. for 25 mins. After incubation, cells were rinsed with PBS to remove dye and Fluorescence was measured at 528/590 for red and 485/528 for green. Ratio of Red: green (healthy cells) was calculated.

### Calculations

Increase of Mitochondrial membrane potential was calculated wrt t-BH1P damage control. [(A−B)/B]*100; Where A=Ratio of Red: Green in Test Item treated cells, B=Ratio of Red Green in t-BH1P damage control cells

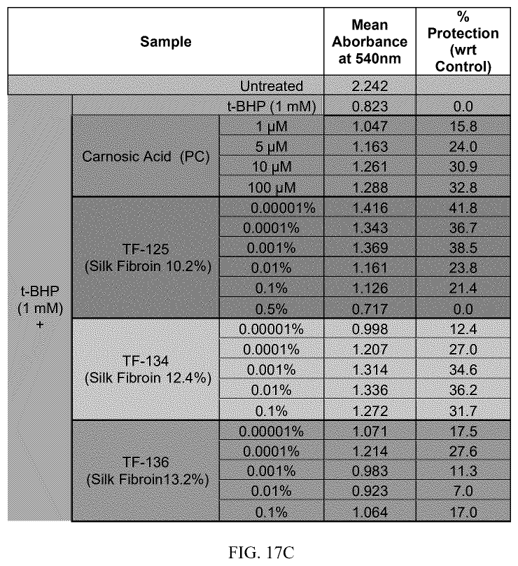

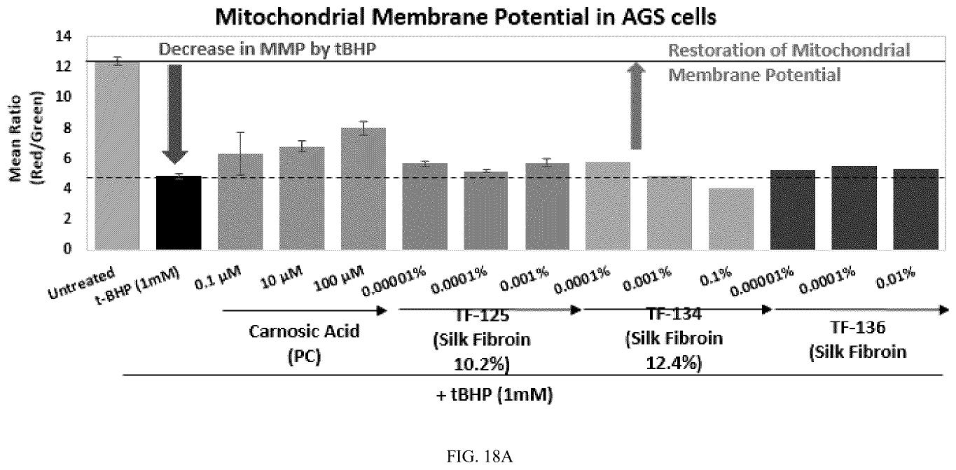

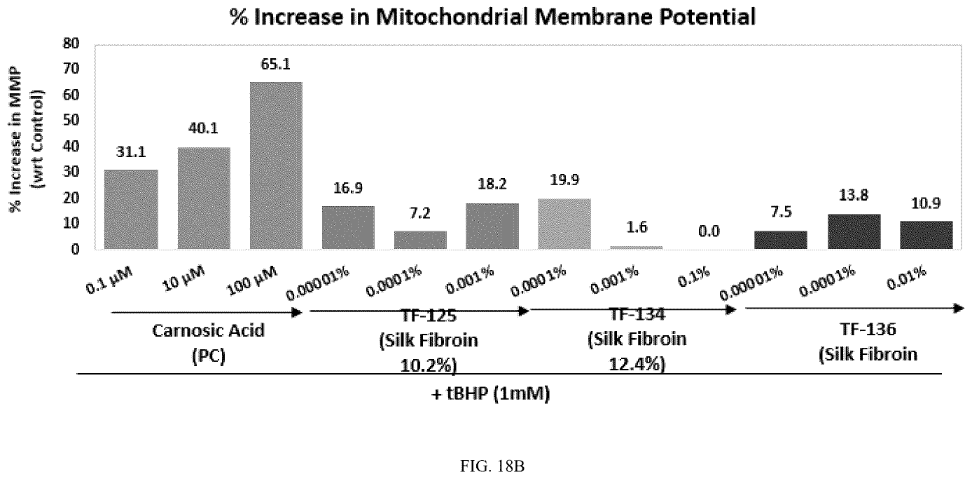

Results: seen at FIG. 18.

3b)—Cytoprotective Effect Against Oxidative Stress in Gastric Cells (AGS) by Antiapoptotic Effect (ROS)

### Study Design

Test System—Human adenocarcinoma gastric cell line (AGS)
Number of cells plated—10,000 cells/well
FBS concentrations—0.5%
Test items—TF-125(10.2%), TF-134(12.4%), TF-136 (13.2%)
Time points—48 h pretreatment, t-BHP (1 mM)
Estimation method—ROS levels by DCFDA dye
Positive Control—Carnosic Acid

### Procedure

Cells were plated in 10% FBS in 96-well plates and incubated for 24 hours. Cells were then treated with Test items in 0.5% FBS at various concentrations for 24 hours. After 48 hours, damage was induced using t-BHP (1 mM) damage in cells. After 3.5 hours, the effect on ROS generation was determined by DCFDA assay. After incubation, medium from each well was removed. 100 μl of 10 μM DCFDA dye in phenol-free medium was added to each well and cells were incubated at 37° C. for 45 minutes. After incubation, cells were rinsed with PBS to remove dye and Fluorescence was measured at 485/528. Decrease in ROS generation was calculated wrt t-BHP damage control.

### Calculations

Decrease in ROS generation was calculated wrt UVB damage control. [(B−A)/B]*100; Where A=RFU of Test Item treated cells, B=RFU of t-BHP damage control cells

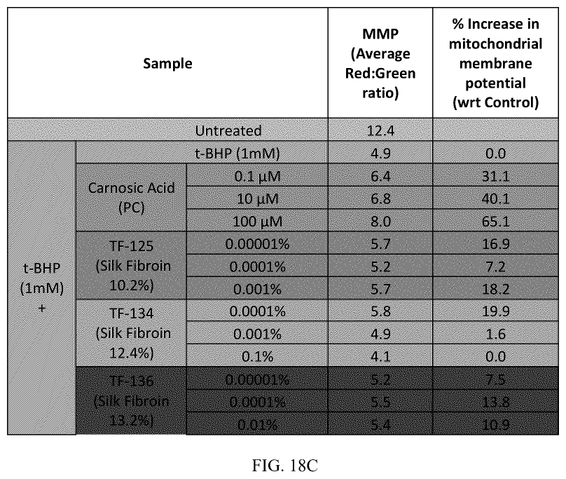

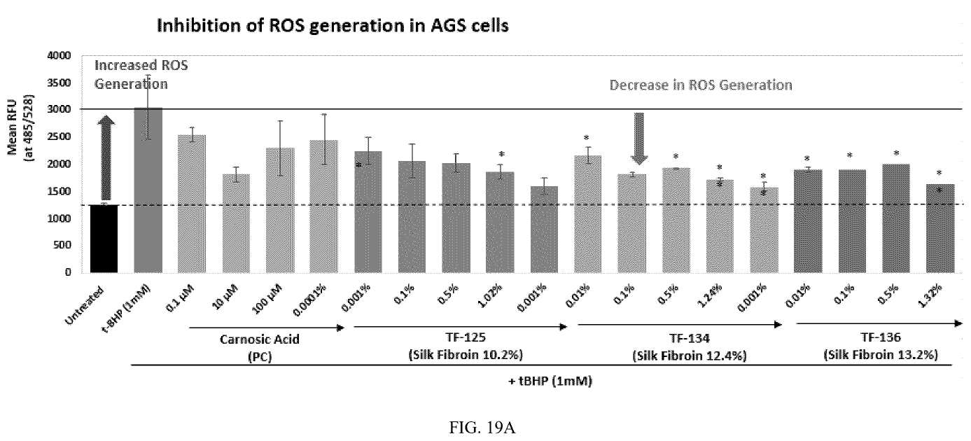

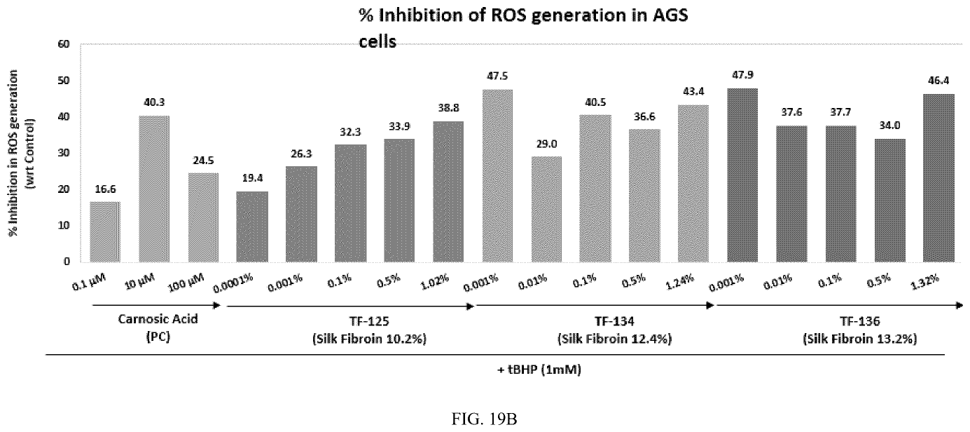

Results: seen at FIG. 19.

Findings: The above data shows that in the presence of oxidative stress/damage the silk fibroin peptides according to the disclosure improve gastric cell viability and reduce gastric cell death leading to a cytoprotective effect suggesting a protective effect with direct treatment to the gastric cells.

### H. IMMUNITY Immunostimulatory Activity by Increase in Levels of Cytokines (TNF-α, IL-6, IL-8/IFN-γ, MIP-1-α, IL-1-β) In Immune Cells

The immune system is a complex network of organs, cells and proteins that defends the body against infection, whilst protecting the body's own cells. The immune system is the body's tool for preventing or limiting infection Its complex network of cells, organs, proteins, and tissues enable it to defend the body from bacteria, viruses, parasites, and more.

A fully functional immune system can distinguish healthy tissue from unwanted substances If it detects an unwanted substance, it will mount an immune response a complex attack to protect the body from invaders It also recognizes and removes dead and faulty cells.

The immune system does not always get it right, however, sometimes, for instance, it is unable to fight effectively because a person has a health condition or needs certain medications that affect how the system works. In autoimmune diseases and allergies, the immune system mistakenly perceives healthy tissue as unhealthy and launches an unnecessary attack, leading to uncomfortable and sometimes dangerous symptoms.

The immune system consists of many parts that work together to defend the body against invaders The primary parts of the immune system include the bone marrow and thymus The bone marrow is extremely important to the immune system because all the body's blood cells (including T and B lymphocytes) originate in the bone marrow B lymphocytes remain in the marrow to mature, while T lymphocytes travel to the thymus. The antigen presenting cells (dendritic cells) act as sentinels for the foreign pathogens Upon encountering, these cells activate T cells that produce cytokines for immune response and B cells that produce antibodies.

Cytokines are small proteins that are crucial in controlling the growth and activity of other immune system cells and blood cells. When released, they signal the immune system to do its job. Cytokines affect the growth of all blood cells and other cells that help the body's immune and inflammation responses. Cytokines participate in many physiological processes including the regulation of immune and inflammatory responses. These effector molecules are produced transiently and locally controlling the amplitude and duration of the response. A variety of experiments has shown that excessive or insufficient production may significantly contribute to the pathophysiology of a range of diseases.

Increased risk of developing autoimmune manifestations has been identified in different primary immunodeficiencies (PIDs). In such conditions, autoimmunity and immune deficiency represent intertwined phenomena that reflect inadequate immune function. Immunostimulants, also known as immune stimulators, are substances (drugs and nutrients) that stimulate the immune system by inducing activation or increasing activity of any of its components. This class of compounds modulate the immune system by increasing the host's resistance to disease [Immunostimulants are biologically active substances obtained from natural or synthetic sources with different chemical characteristics and mechanism of action that modulate the immune system of host to increase resistance against various infections. They interact with specific receptors and cellular components of innate and adaptive response to modulate the immune response. They are used during suppressed immunity condition like cancer disease, AIDS, SARS etc. to improve the host's resistance. Immunostimulants are classified into two categories: specific and nonspecific immunostimulants.

Immunostimulants function by the recognition of toll like receptors which is a pathogen associated molecular model, in the innate immune cells thereby initiating an immune response, also, ingestion of cytokines can trigger the response of the immune cells.

The immune system has evolved to protect the host from a universe of pathogenic microbes that are themselves constantly evolving. The immune system also helps the host eliminate toxic or allergenic substances that enter through mucosal surfaces. When a body lacks sufficient white blood cells or antibodies, the immune system is considered suppressed. A suppressed immune system is weakened to the point that it cannot mount a proper immune response to protect the body from pathogens, such as bacteria, viruses, and other infectious microorganisms. Therefore, people with a suppressed immune system become vulnerable to various infectious diseases [12]. To restore the immune function, immune stimulators are widely used. Immunostimulants, also known as immune stimulators, are substances (drugs and nutrients) that stimulate the immune system by inducing activation or increasing activity of any of its components. This class of compounds modulate the immune system by increasing the host's resistance to disease. Immune cells such as Dendritic cells act as Antigen presenting cells and play key role in mounting an immune response. Splenocytes are a mixture of immune cells such as T cells, B cells, NK cells, that secrete cytokines pivotal in immunity. Human monocytes also play important role in generating an immune response.

Due to their prime importance in generation of an immune response, Dendritic cells [13-15], Splenocytes [16-18] and THP-1 cell line (human monocytes) [19-21] are widely reported as model systems to screen immunostimulatory activity of test agents. Increase in the secretion of cytokines by these cell types from basal untreated levels is employed as primary endpoint for immuno-stimulation.

### Study Design

Test System—Murine Splenocytes isolated from male C57BL/6 mouse (8-9 weeks)
Number of cells plated—2 million cells/well in 24 Well plate
FBS concentrations—10%,
Test items—TF-125 (10.2%), TF-134 (12.4%), TF-136 (13.2%)
Time point—24 h treatment
Estimation method—ELISA
Positive Control—LPS (Lipopolysaccharide)

### Procedure

Spleens were aseptically excised from male C57BL/6 mice and a single cell suspension was prepared. Cells were pelleted at 1200 rpm for 8 minutes. Erythrocytes in the splenocytes were removed by treatment with lysis buffer (0.15 M NH4Cl, 0.01 M KHCO3, and 0.1 mM Na2EDTA, pH 7.4). After lysis of RBCs, cells were washed twice in RPMI-1640 medium by centrifugation at 1200 rpm for 8 minutes and used for experiments. Cells were plated in 10% FBS in 24-well plates. Cells were treated with 3 Test items at non-cytotoxic concentrations for 24 hours. After 24 hours, culture supernatants were collected. Levels of cytokines (TNF-α, IL-6, IFN-γ, MIP-1α, IL-1β) were determined using ELISA as follows: Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm.

### Calculations

Fold increase in each sample was calculated wrt control (Untreated cells). [(A−B)/B]*100; Where A=Conc of cytokine in of Test Item treated cells, B=Conc of cytokine in Control (Untreated) cells.

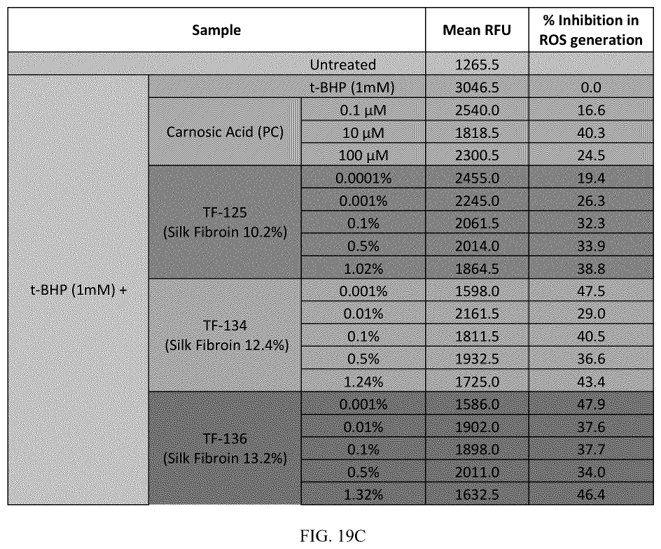

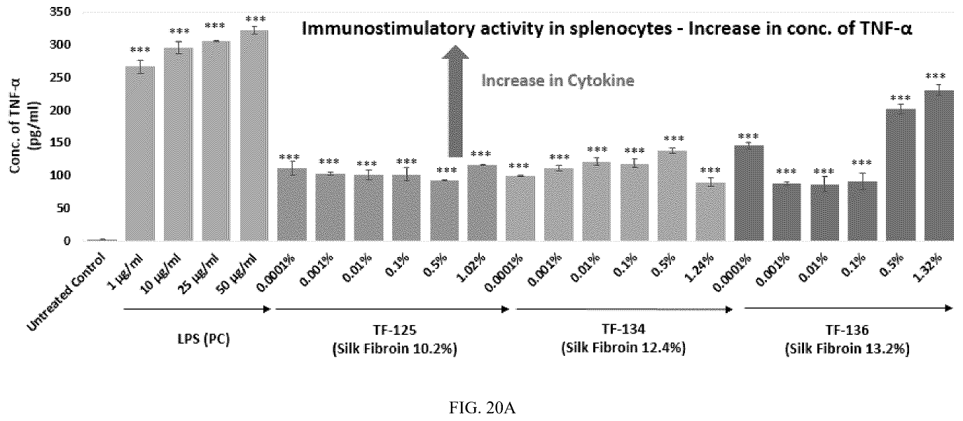

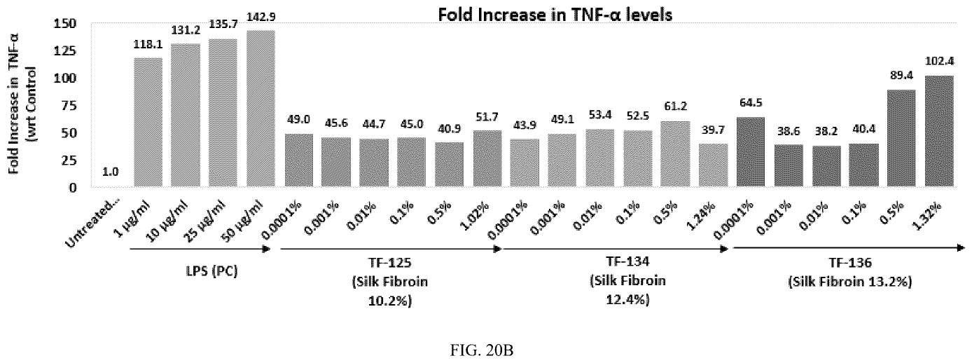

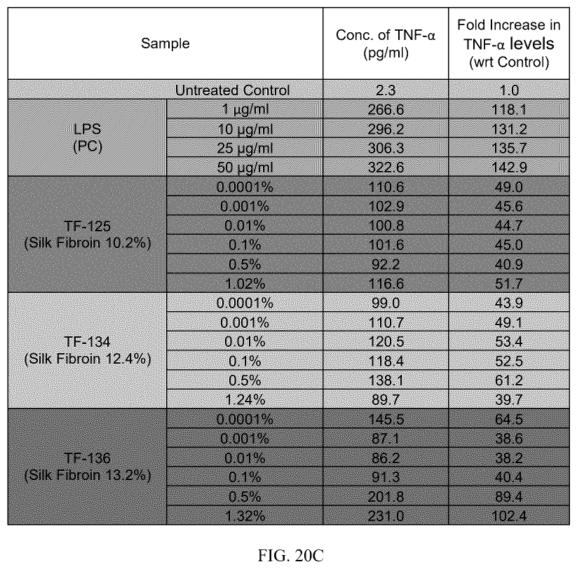

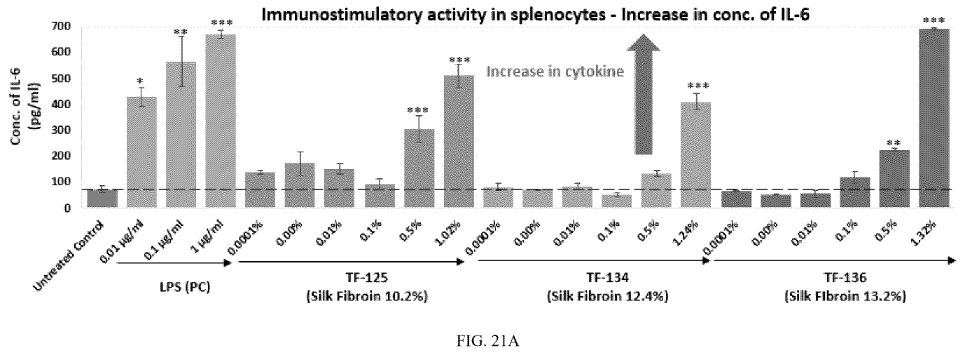

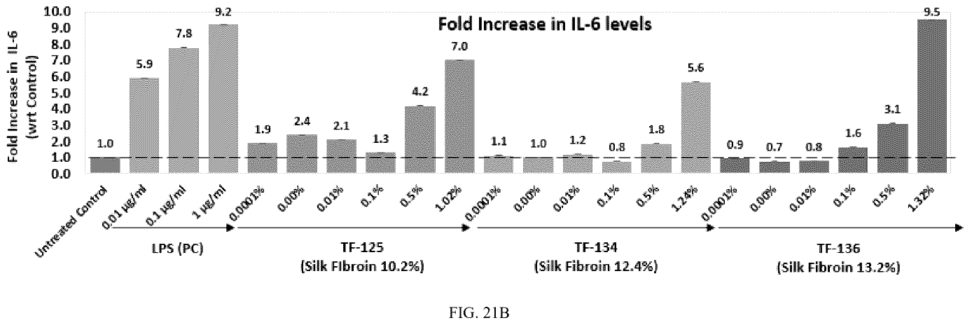

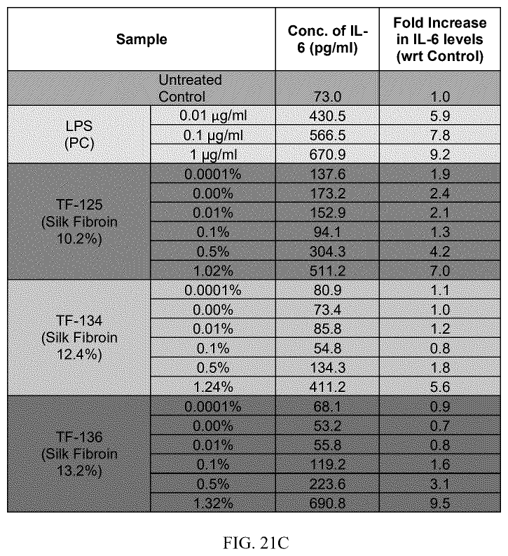

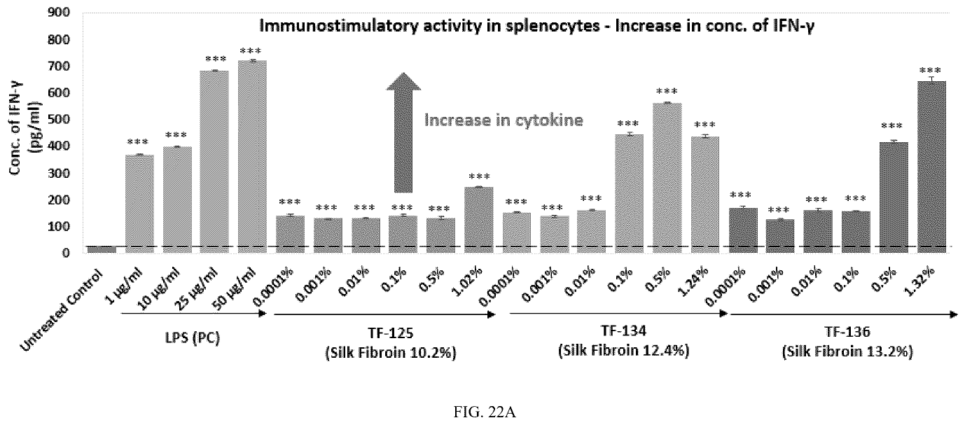

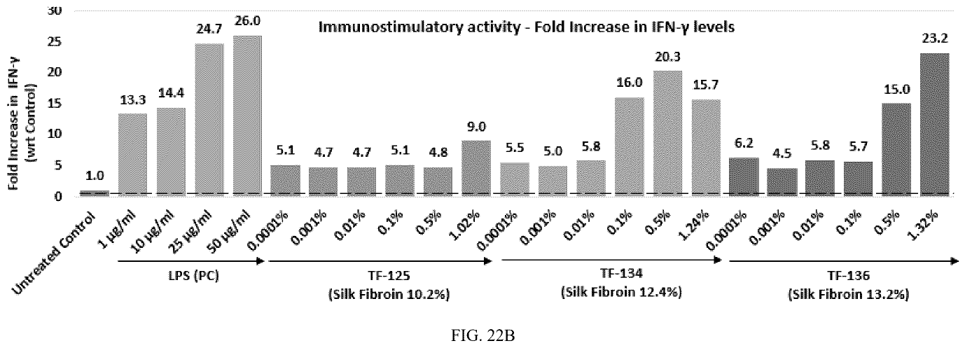

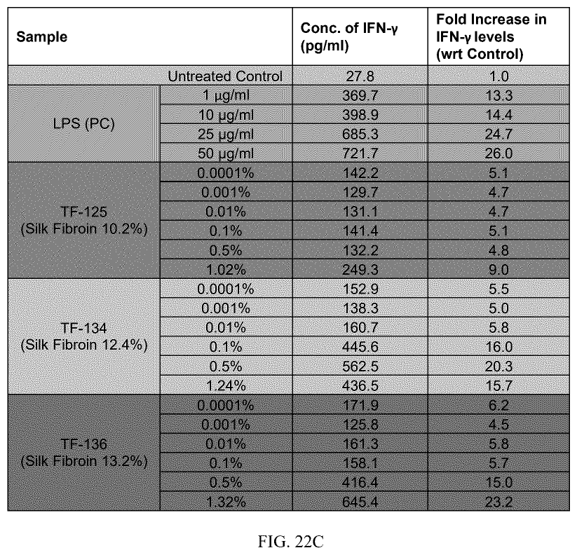

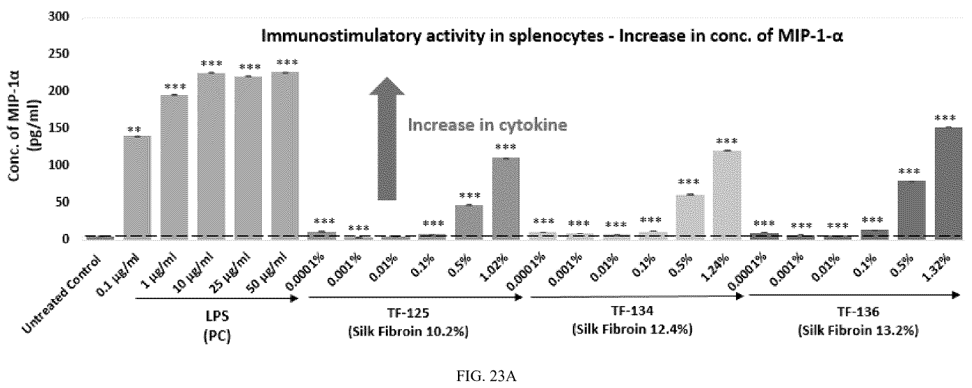

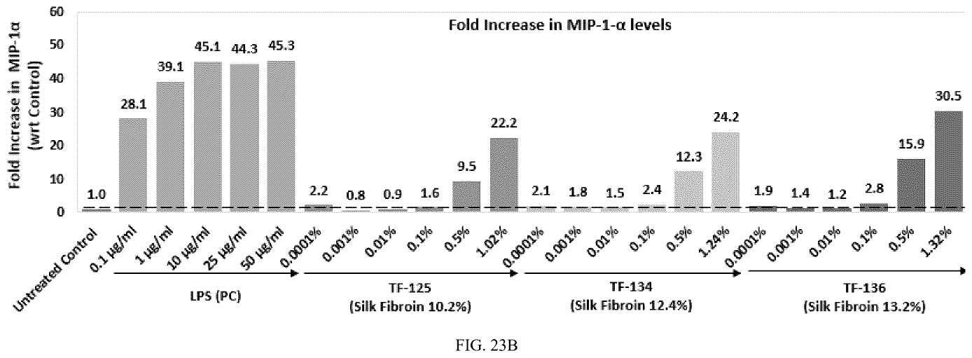

Results: seen at FIGS. 20-23.

### I. Immunostimulatory Activity—Increase in Levels of Cytokines in Murine BMDCs

### Study Design

Test System—Murine Bone Marrow Derived Dendritic Cells (BMDC), BM cells isolated from male C57BL/6 mouse (7-8 weeks)
Number of cells plated—0.2 million cells/well in 24 Well plate
FBS concentrations—0%
Test items—TF-125 (10.2%), TF-134 (12.4%), TF-136 (13.2%)
Time point—24 h treatment
Estimation method—ELISA.
Positive Control—LPS (Lipopolysaccharide)

### Procedure

Murine Bone marrow cells were isolated and differentiated into dendritic cells using 20 ng/ml rmGMCSF for 6 days in 90 mm Petri dishes, at a density of 2×106 cells/plate. At day-3 media was replenished by adding 10 ml of fresh growth medium containing rmGMCSF 20 ng/ml. On 6th day, cells were harvested and flushed using media, counted and plated. Cells were plated in 10% FBS in 24-well plates and incubated for 24 hours. Cells were serum starved in 0% FBS. Cells were treated with 3 Test items in 0% FBS at non-cytotoxic concentrations for 24 hours.

After 24 h, culture supernatants were collected. Levels of cytokines (TNF-α,IL-6, IFN-γ, MIP-1α, IL-1β) were determined using ELISA as follows-Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm.

### Calculations

Fold increase in each sample was calculated wrt control (Untreated cells).

[(A−B)/B]*100; Where A=Conc of cytokine in of Test Item treated cells, B=Conc of cytokine in Control (Untreated) cells.

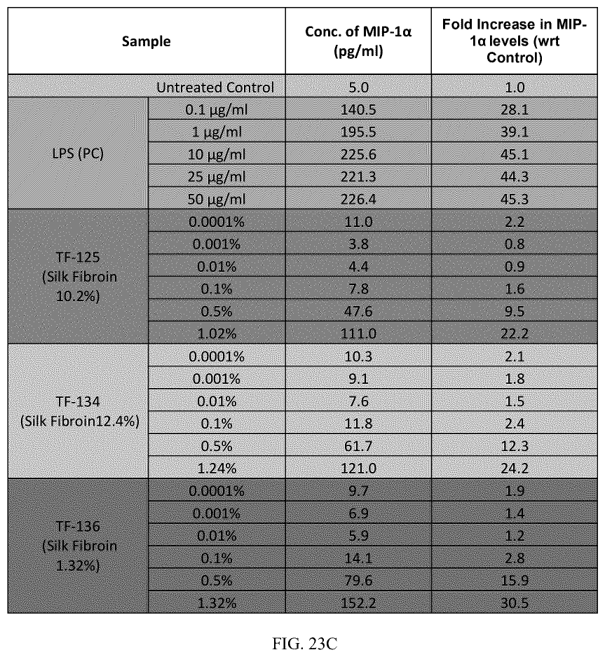

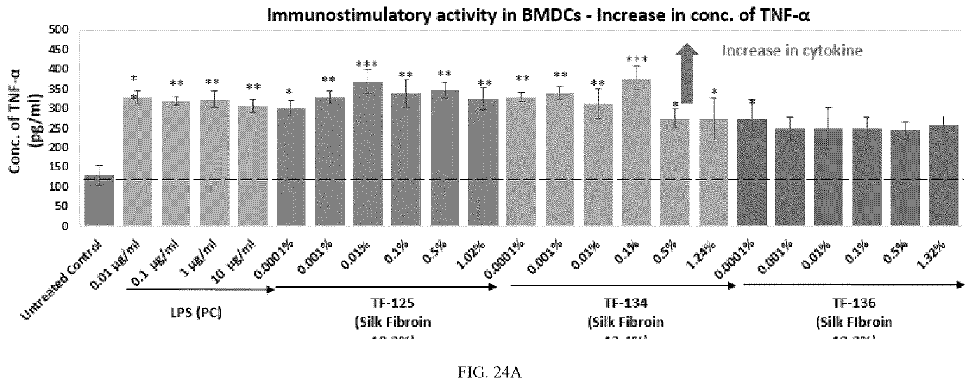

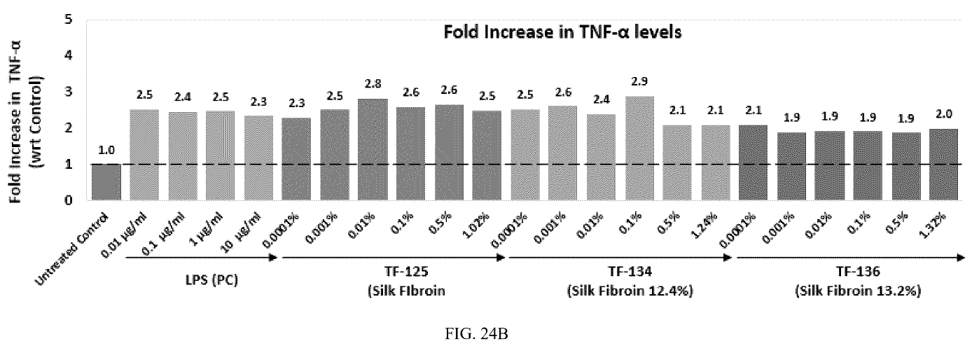

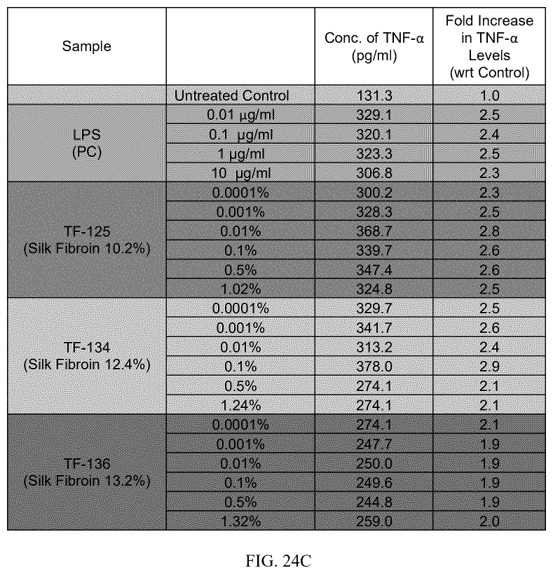

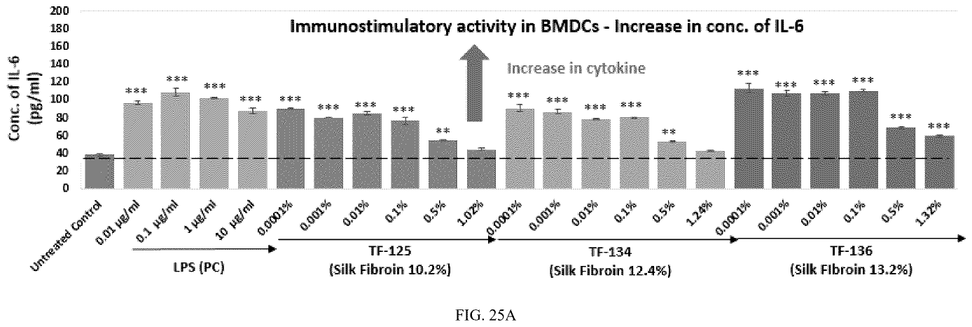

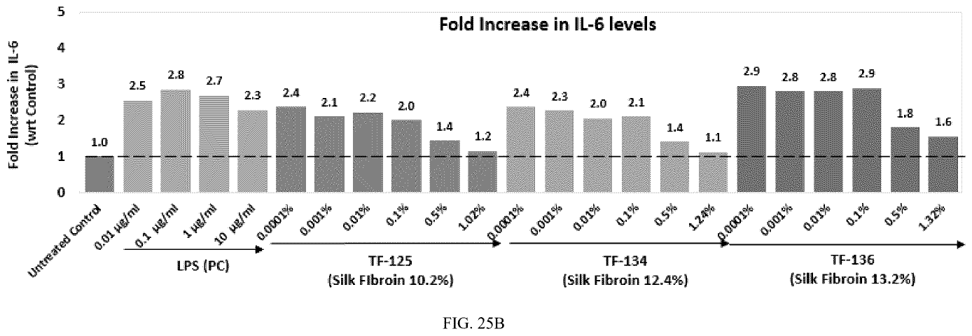

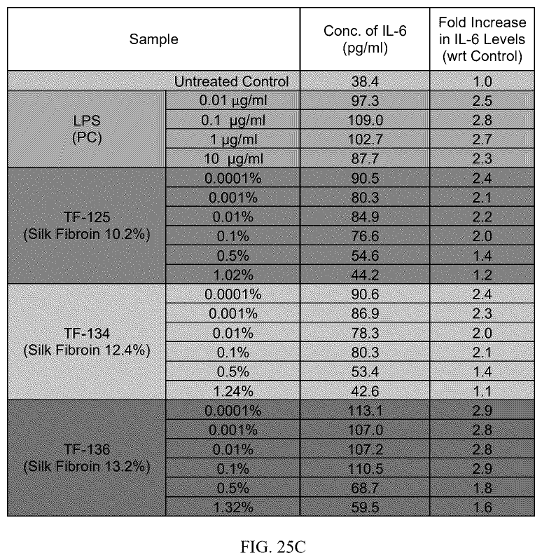

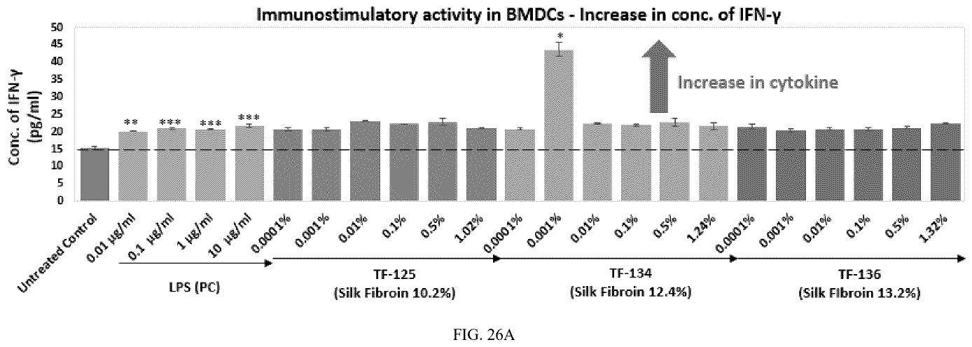

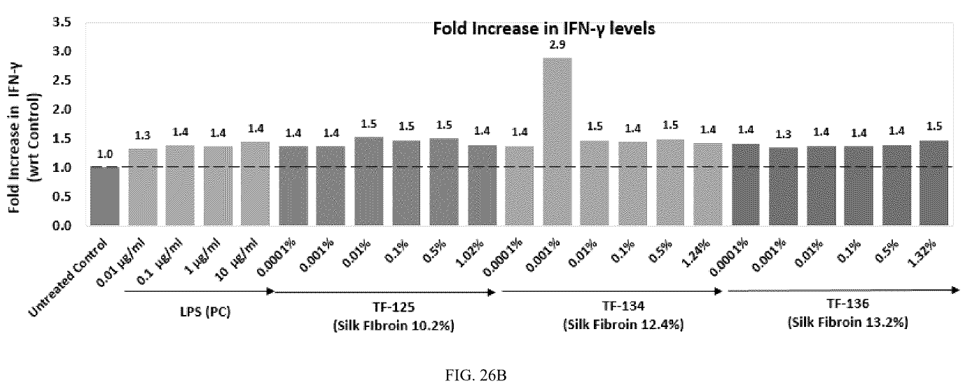

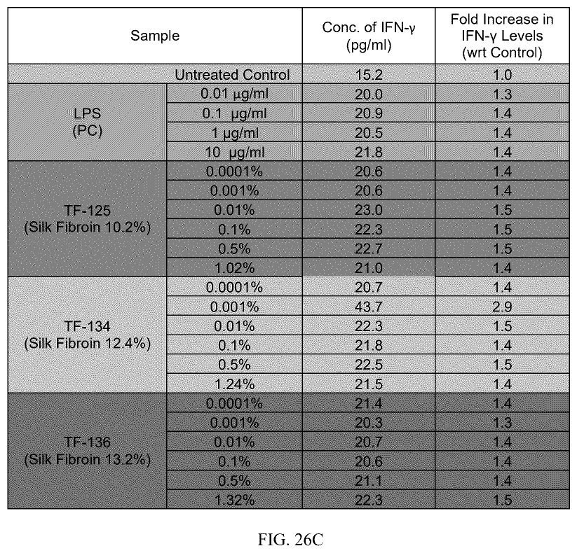

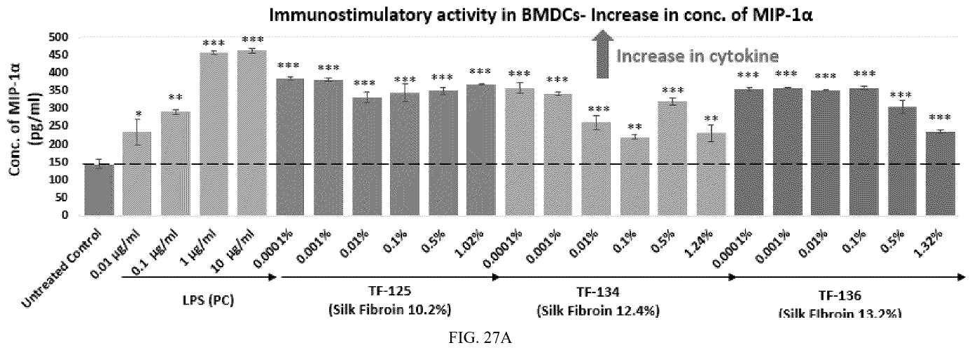

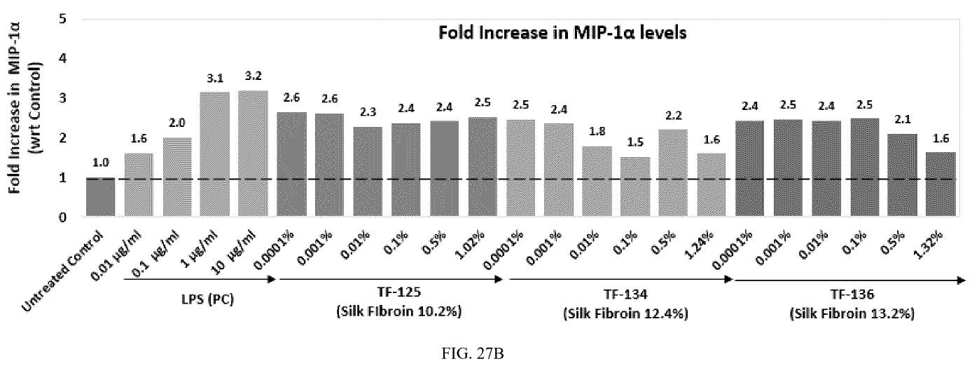

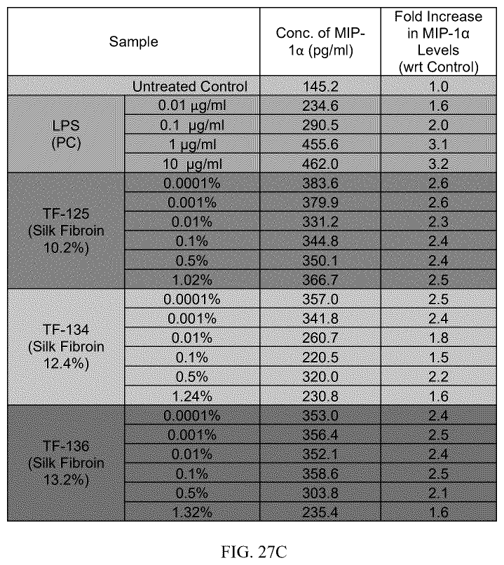

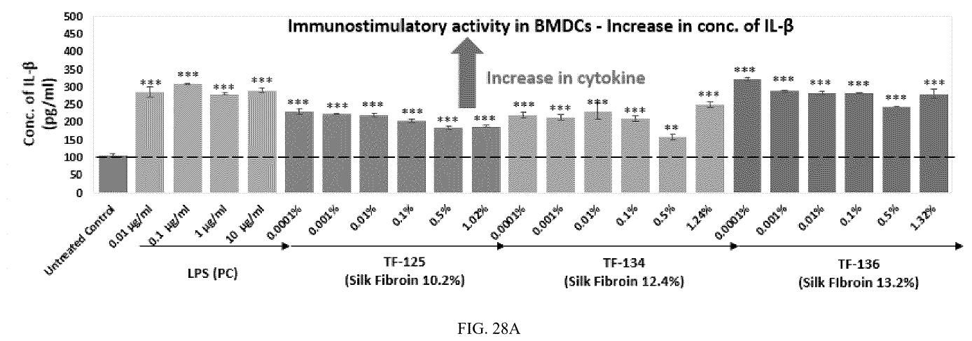

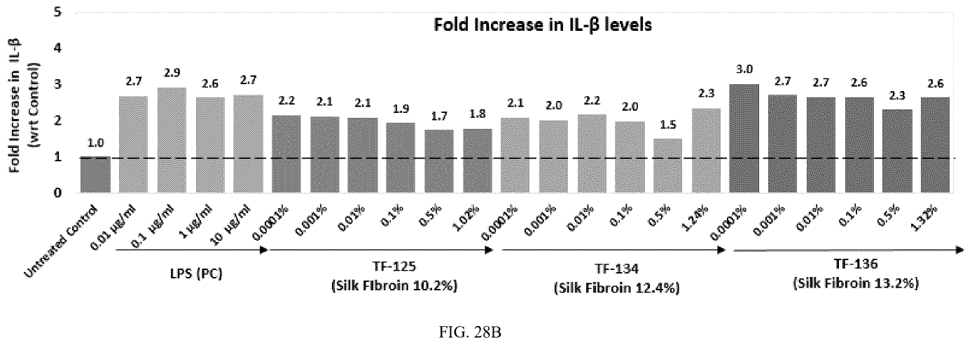

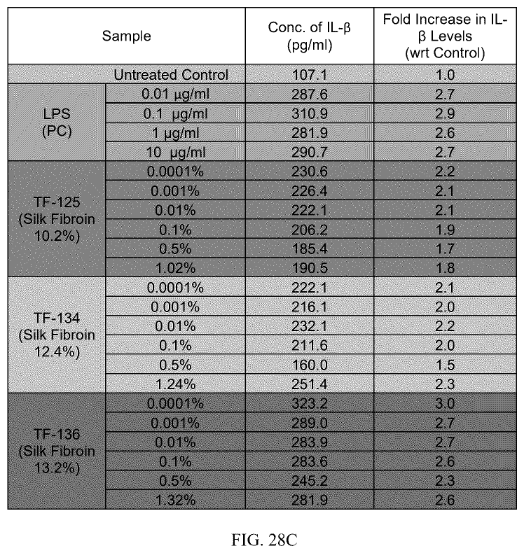

Results: seen at FIGS. 24-28.

### J. Immunostimulatory Activity—Increase in Levels of Cytokines in Human Monocytic Cells (THP-1)

### Study Design

Test System—Human Monocytic Cells (THP-1)
Number of cells plated—0.1 million cells/well in 24 Well plate
FBS concentrations—0%,
Test items—TF-125 (10.2%), TF-134 (12.4%), TF-136 (13.2%)
Time points—48 h treatment
Estimation method—ELISA.
Positive Control—LPS (Lipopolysaccharide)

### Procedure

Cells were plated in 10% FBS in 24-well plates and incubated for 24 hours. Cells were treated with 3 Test items in 0% FBS at non-cytotoxic concentrations for 48 hours. After 48 hours, culture supernatants were collected. Levels of cytokines (TNF-α, IL-6, IL-8, MIP-1α, IL-1β) were determined using ELISA as follows: Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 minutes at RT in dark. The reaction was stopped by adding Stop solution to each well. The optical density of the color was measured at 450 nm.

### Calculations

Fold increase in each sample was calculated wrt control (Untreated cells). [(A−B)/B]*100; Where A=Conc of cytokine in of Test Item treated cells, B=Conc of cytokine in Control (Untreated) cells.

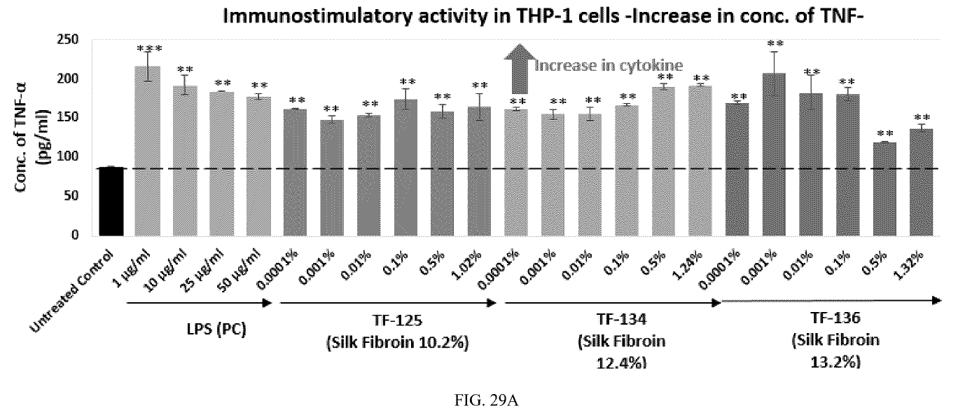

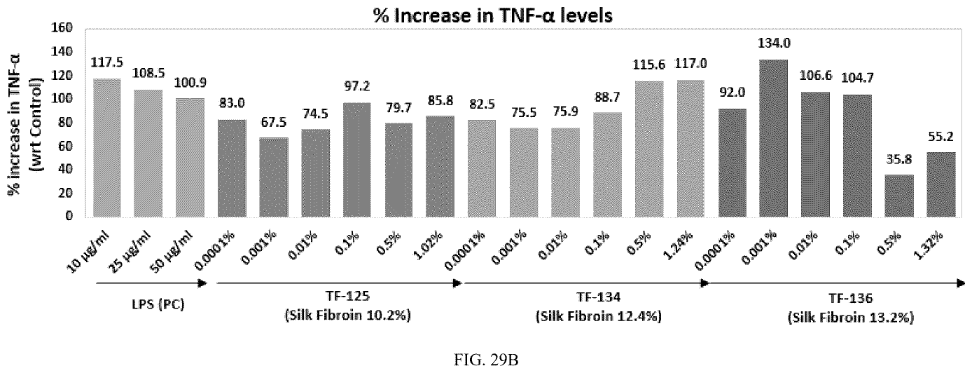

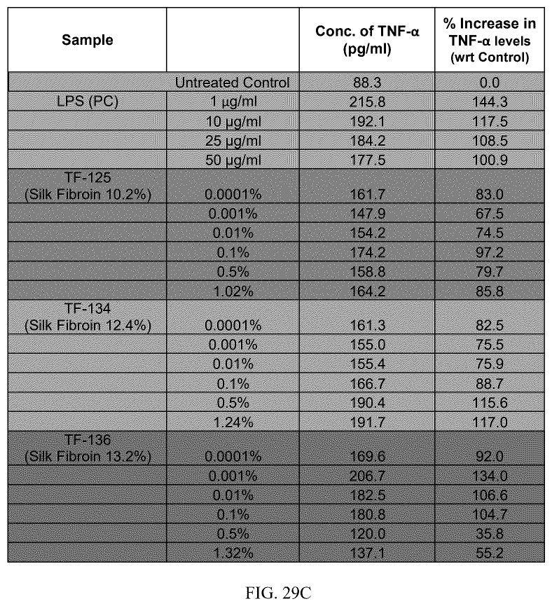

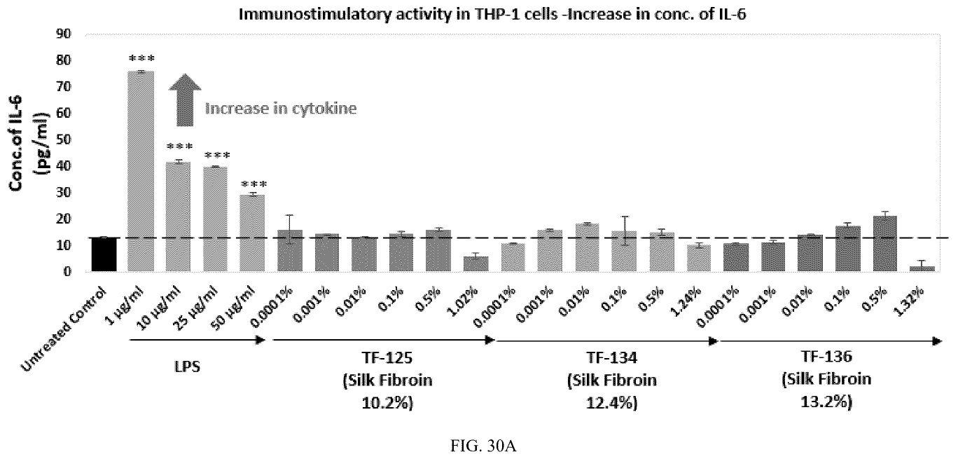

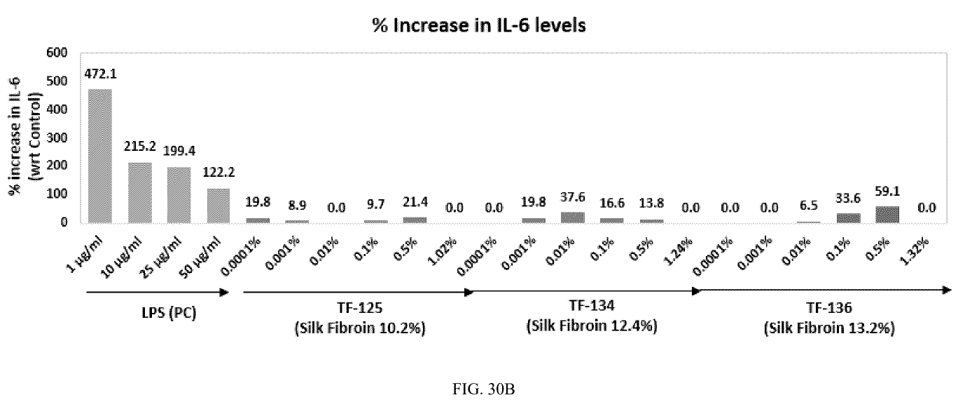

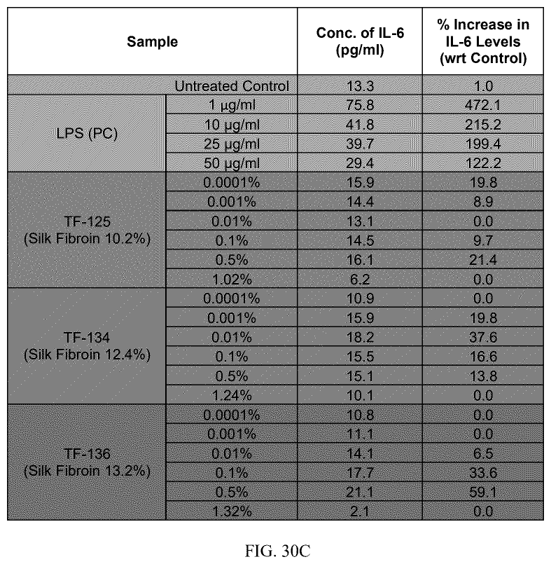

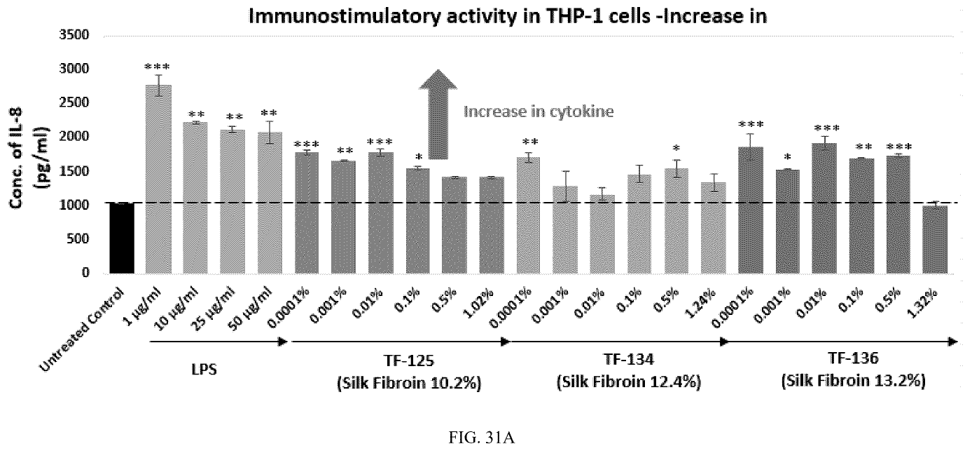

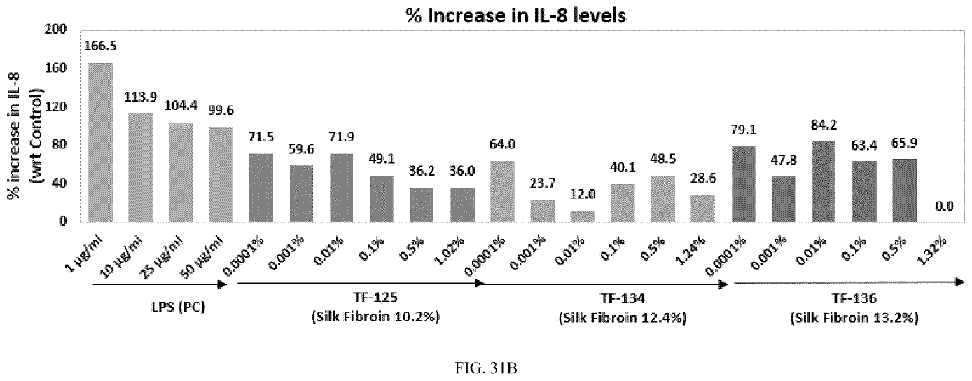

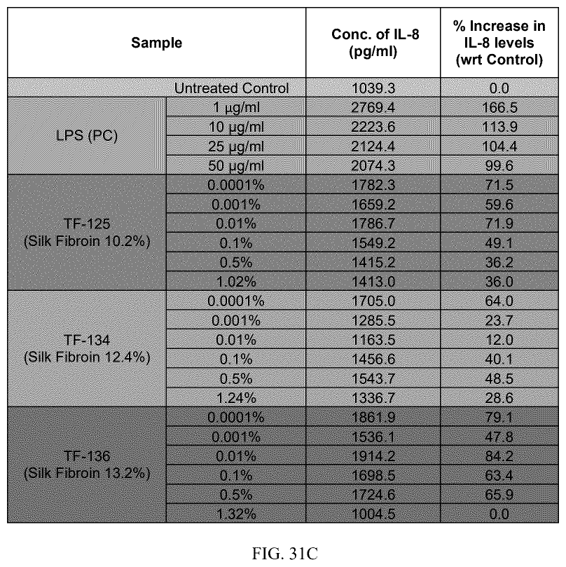

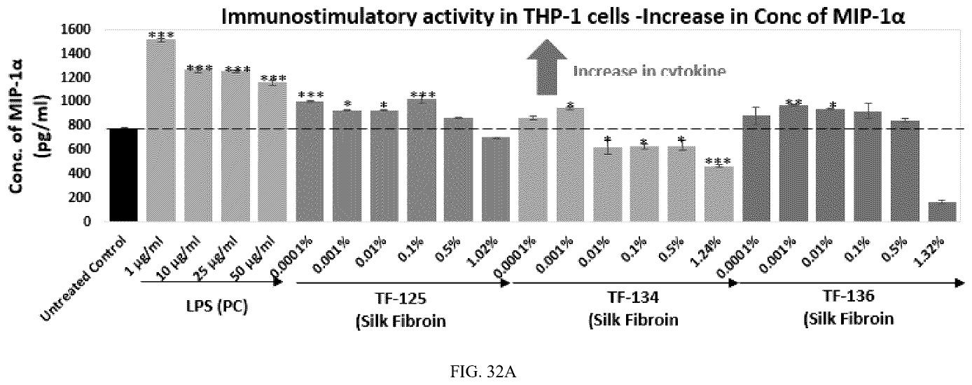

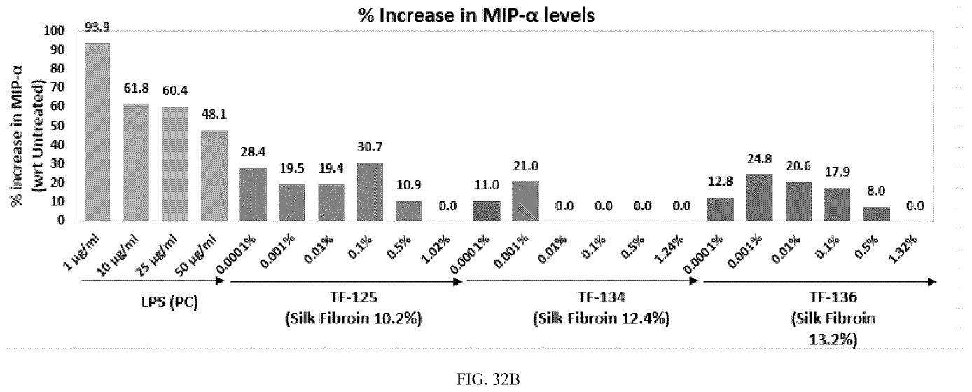

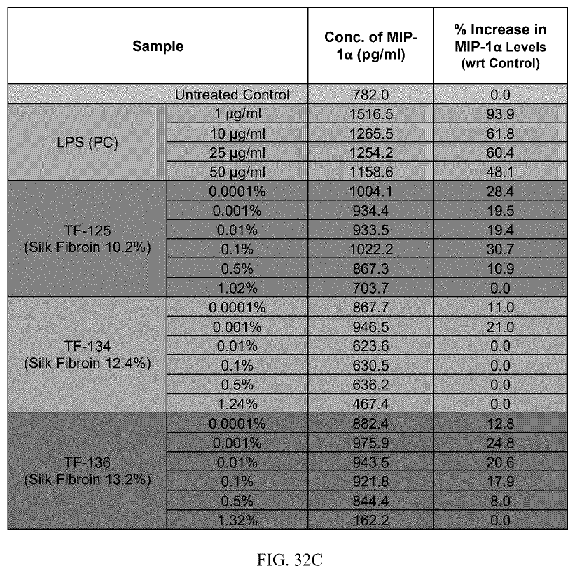

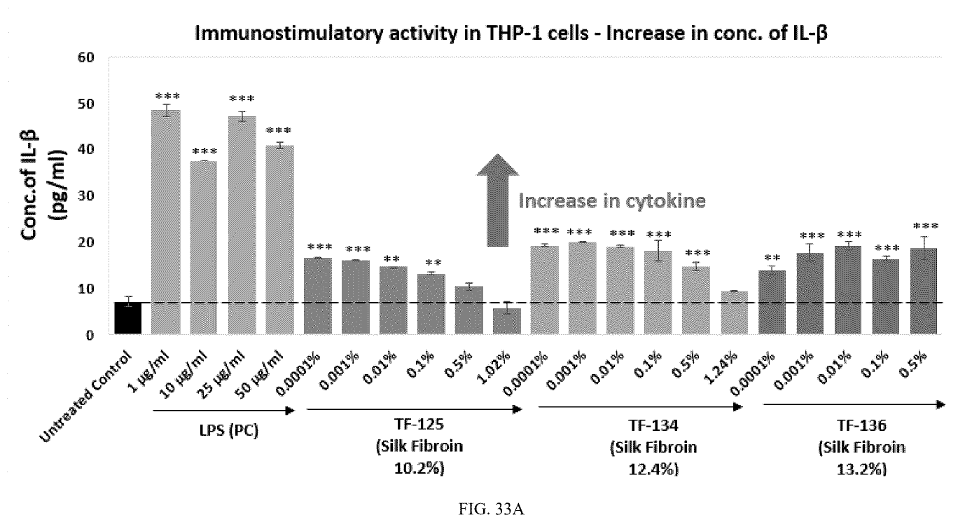

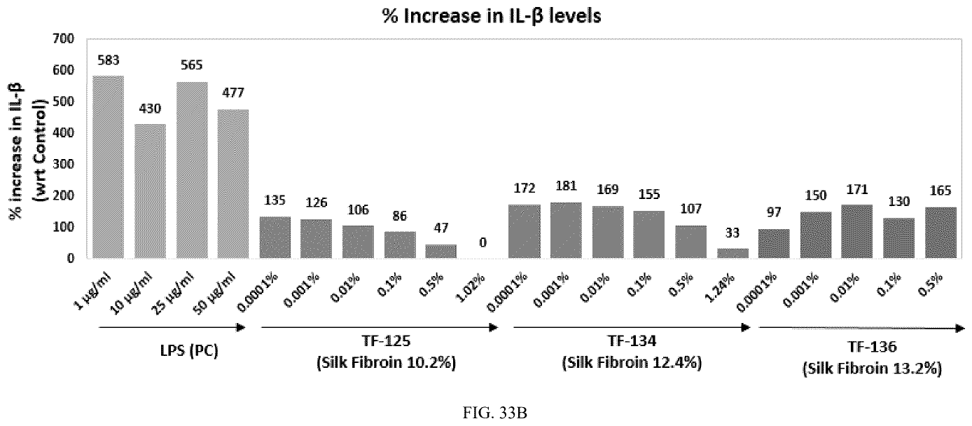

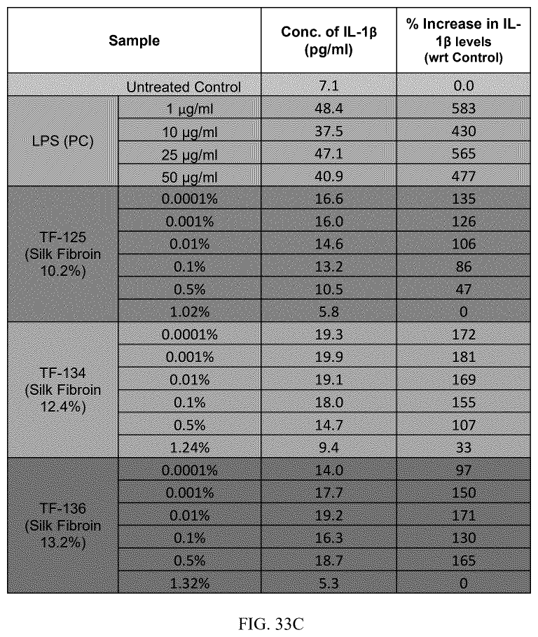

Results: seen at FIGS. 29-33.

Findings: The above data shows that immune cells (mouse and human) are stimulated from their base, untreated level to produce cytokines suggesting that the silk peptides according to the disclosure stimulates the immune system strengthening its response.

### H. SYSTEMIC INFLAMMATION Anti-Inflammatory Activity by Inhibition in Levels of Cytokines (TNF-α, IL-6, IL-8/IFN-γ, MIP-1-α, IL-1-β) in Immune Cells

Inflammation is part of the complex biological response of body tissues to harmful stimuli, such as pathogens, damaged cells, or irritants and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair.

Massive systemic inflammation: the loss of regulation and balance of the pro-inflammatory response results in a massive reaction manifested as the systemic inflammatory response with (a) progressive endothelial dysfunction, (b) platelet sludging blocking the microcirculation, (c) activation of the coagulation system and (d) profound vasodilatation, fluid transition, and maldistribution of blood flow may result in severe shock.

Chronic systemic inflammation (SI) is the result of excessive release of pro-inflammatory cytokines from immune-related cells and the chronic activation of the innate immune system. It can contribute to the development or progression of certain conditions such as cardiovascular disease, cancer, diabetes mellitus, chronic kidney disease, non-alcoholic fatty liver disease, autoimmune and neurodegenerative disorders, and coronary heart disease.

Various anti-inflammatory agents are employed to combat the systemic inflammation by targeting inflammatory cascades involving cytokines and other immune markers.

Several causes of low-grade systemic chronic inflammation (SCI) and their consequences have been identified. The most common triggers of SCI include chronic infections, physical inactivity, (visceral) obesity, intestinal dysbiosis, diet, social isolation, psychological stress, disturbed sleep and disrupted circadian rhythm, and exposure to xenobiotics such as air pollutants, hazardous waste products, industrial chemicals and tobacco smoking.

The consequences of SCI include metabolic syndrome, type 2 diabetes, non-alcoholic fatty liver disease (NAFLD), cardiovascular disease, cancer, depression, autoimmune diseases, neurodegenerative diseases, sarcopenia, osteoporosis and immunosenescence.

Systemic inflammation occurs when the immune system is constantly defending the body. Stress, infection, or chronic diseases can put the body in a proinflammatory state. When this happens, the immune system becomes primed and ready to create an inflammatory response. Systemic inflammation (SI) is increasingly studied in several species because it may be central in many metabolic disturbances and be a risk factor for clinical disease.

Chronic systemic inflammation (SI) is the result of release of pro-inflammatory cytokines from immune-related cells and the chronic activation of the innate immune system. It can contribute to the development or progression of certain conditions such as cardiovascular disease, cancer, diabetes mellitus, chronic kidney disease, non-alcoholic fatty liver disease, autoimmune and neurodegenerative disorders, and coronary heart disease. Release of pro-inflammatory cytokines and activation of the innate immune system may be the result of either external (biological or chemical agents) or internal (genetic mutations/variations) factors. The cytokine Interleukin 6 and C-reactive protein are common inflammatory markers used to diagnose systemic inflammation risk. Baseline C-reactive protein levels deviate due to natural genetic variation, but significant increases can result from risk factors such as smoking, obesity, lifestyle, and high blood pressure.

Anti-inflammatory agents such as NSAIDs, steroids, supplements and other natural substances help in alleviating the disorders associated with systemic inflammation by targeting overexpression of inflammatory cytokines primarily.

Immune cells such as Dendritic cells act as Antigen presenting cells and play key role in mounting an immune response. Splenocytes are a mixture of immune cells such as T cells, B cells, NK cells, that secrete cytokines pivotal in immunity. Human monocytes also play important role in generating an immune response. Due to their prime importance in generation of an immune response, Dendritic cells, Splenocytes and THP-1 cell line (human monocytes) are widely reported as model systems to screen anti-inflammatory activity of test agents by targeting the hypersecretion of inflammatory cytokines LPS is extensively reported as stimulating agent to induce the inflammation in these cell types. These key cell types are notably sensitive to LPS and respond by expressing many inflammatory cytokines.

### J. Anti-Inflammatory Activity Inhibition of Cytokines in Murine Splenocytes Study Design

Test System—Murine Splenocytes
Number of cells plated—2 million cells/well in 24 Well plate
FBS concentrations—10%,
Test items—TF-125 (10.2%), TF-134 (12.4%), TF-136 (13.2%)
Time points—24 h Co-treatment
Estimation method-Murine IL-6, IFN-γ, MIP-1α, TNF-α, IL-1βELISA.
Positive Control—Dexamethasone

### Procedure

Cells were plated in 10% FBS in 24-well plates. Cells were then treated with 3 Test items at non-cytotoxic concentrations and stimulated with inflammatory stimulus (LPS 50 ng/ml). After 24 hours, culture supernatants were collected. Levels of cytokines (TNF-α, IL-6, IFN-γ, MIP-1α, IL-1β) were determined using ELISA as follows: Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 minutes at RT in dark. The reaction was stopped by adding Stop solution to each well. The optical density of the color was measured at 450 nm.

### Calculations

Percent Inhibition in each sample was calculated wrt control (LPS treated cells). [(B−A)/B]*100; Where A=Test Item treated cells, B=Control (LPS treated) cells.

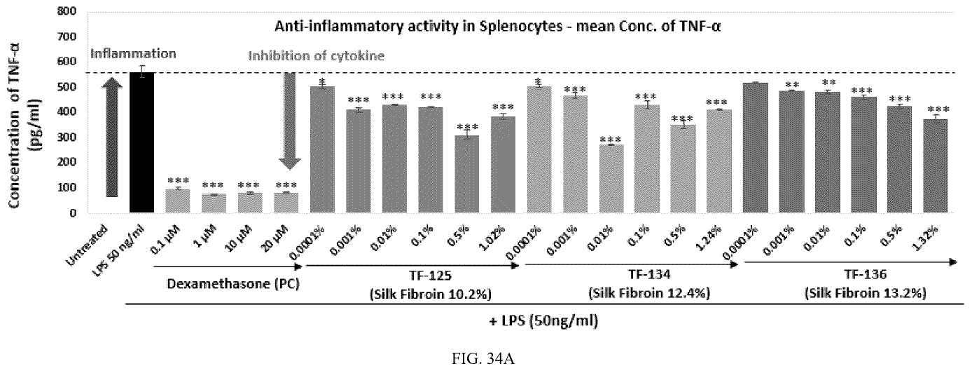

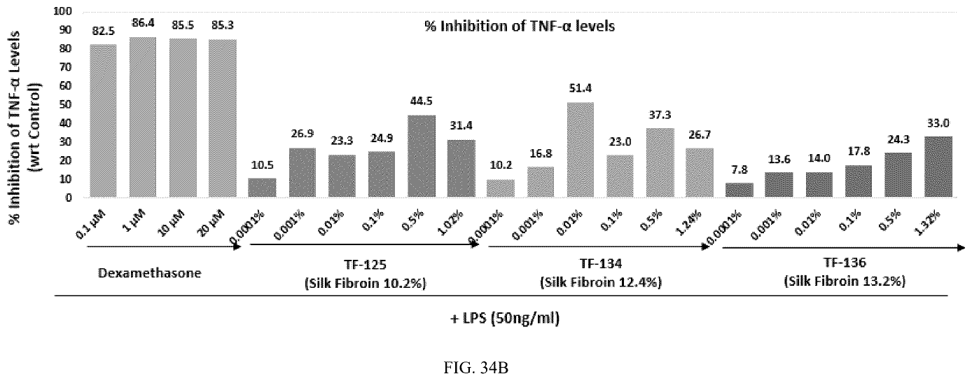

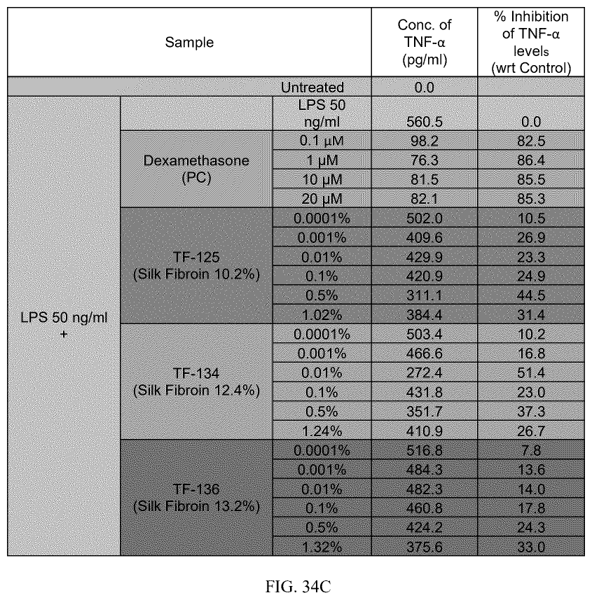

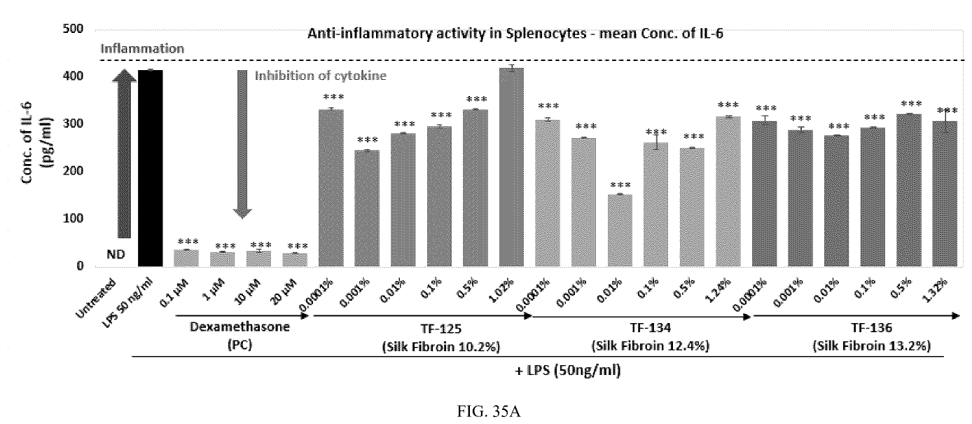

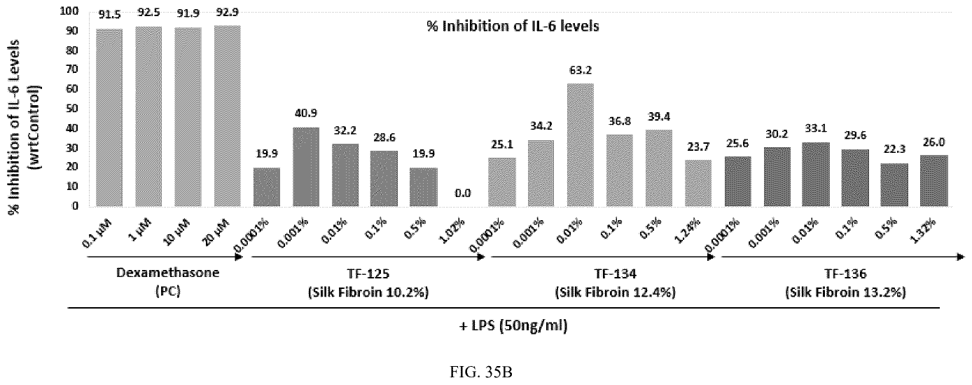

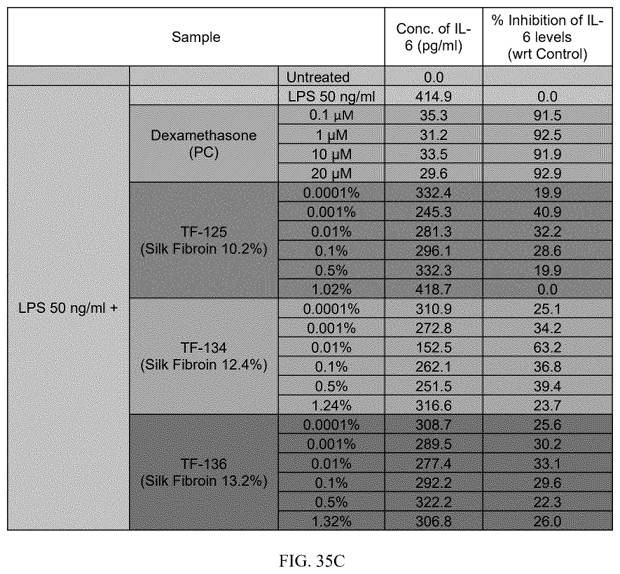

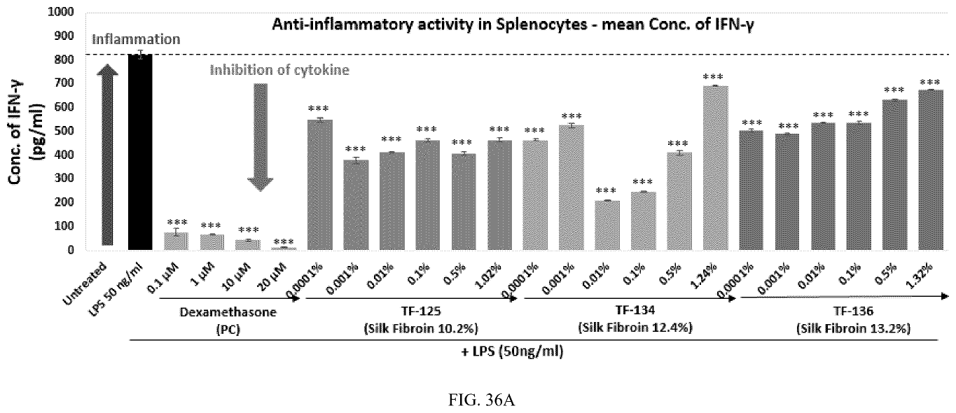

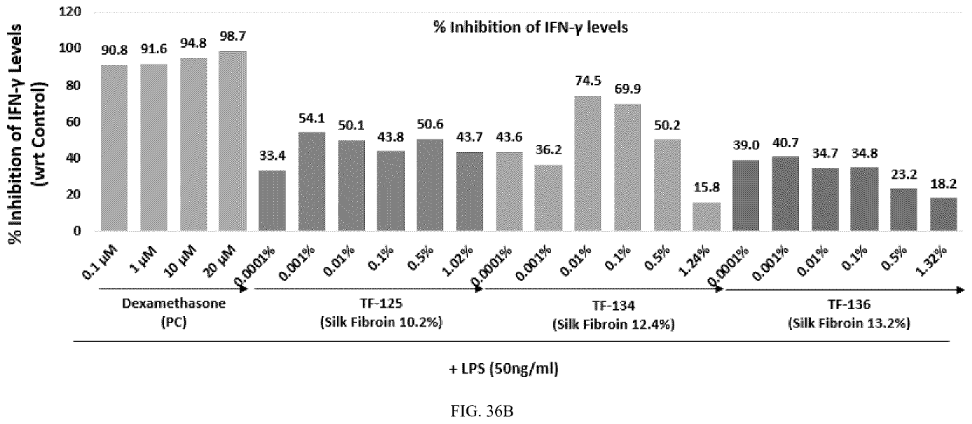

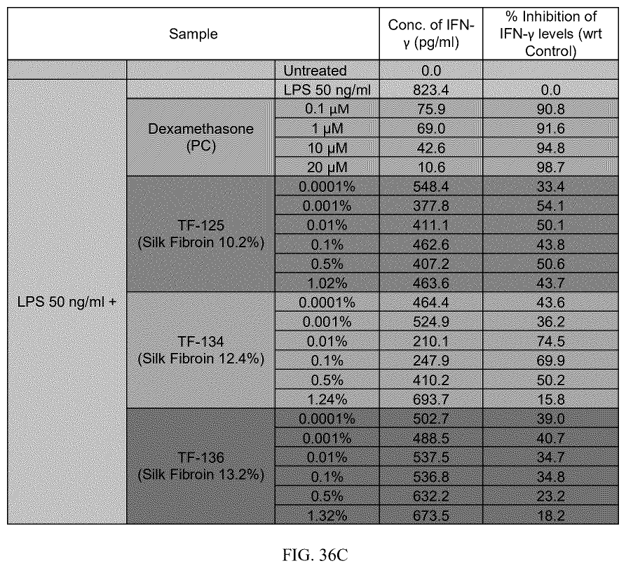

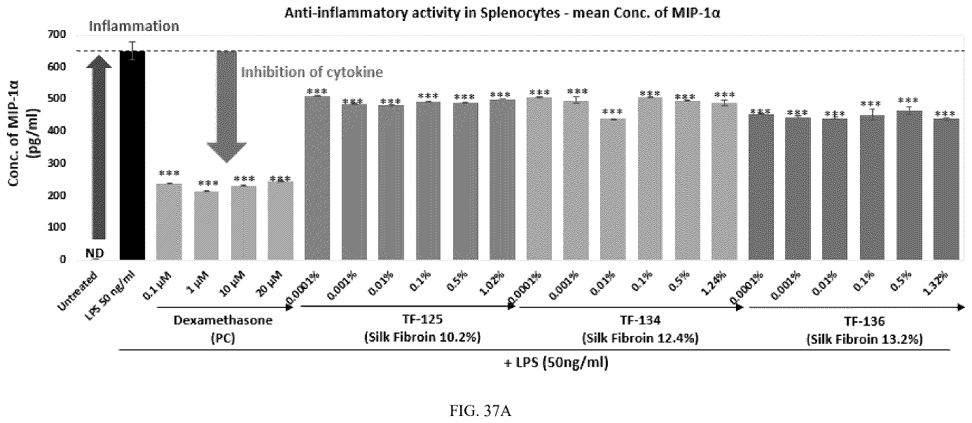

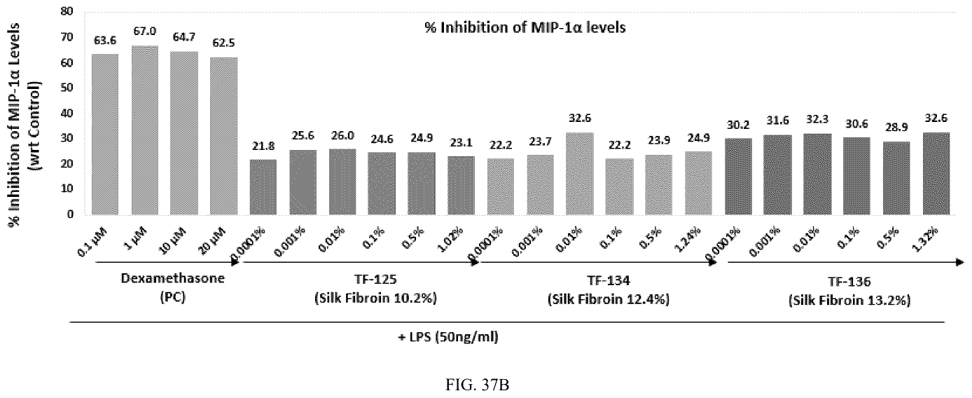

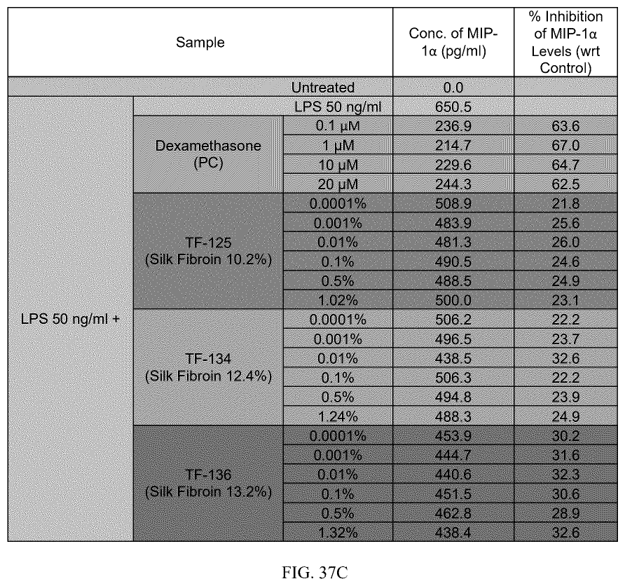

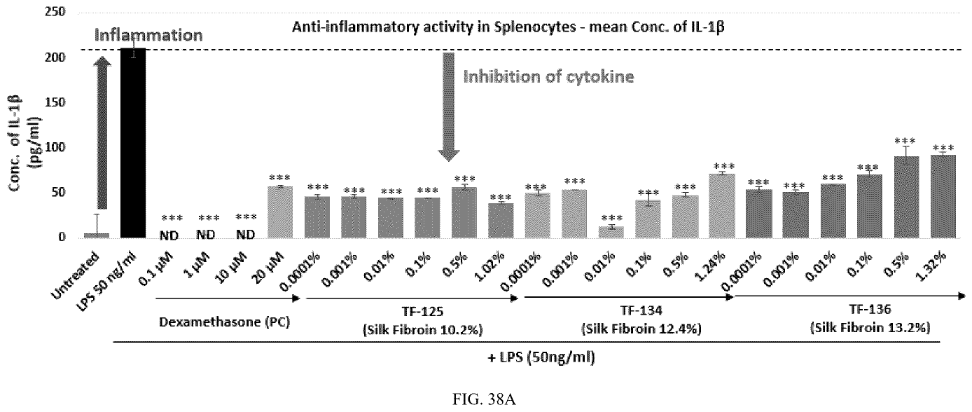

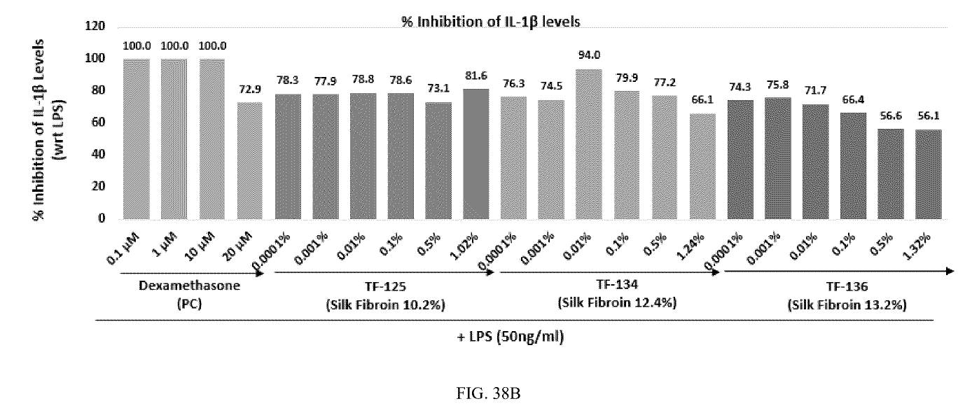

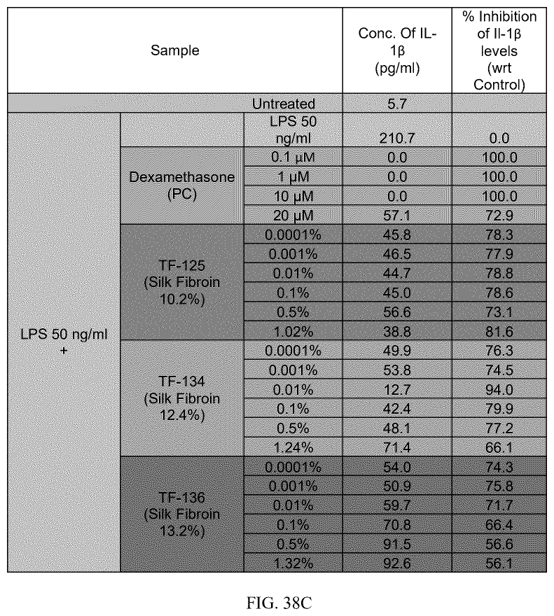

Results: seen at FIGS. 34-38.

### K. Anti-Inflammatory Activity—Inhibition of Cytokines in Murine BMDCs Study Design

Test System—Murine Bone Marrow Derived Dendritic Cells (BMDC)
Number of cells plated—0.2 million cells/well in 24 Well plate
FBS concentrations—0%,
Test items—TF-125 (10.2%), TF-134 (12.4%), TF-136 (13.2%)
Time points—24 h co-treatment
Estimation method—Murine IL-6, IFN-γ, MIP-1α, TNF-α, IL-1βELISA.
Positive Control—Dexamethasone

### Procedure

Cells were plated in 10% FBS in 24-well plates and incubated for 24 hours. Cells were co-treated with 3 Test items in 0% FBS at non-cytotoxic concentrations and stimulated with inflammatory stimulus (LPS 50 ng/ml). After 24 hours, culture supernatants were collected. Levels of cytokines (TNF-α,IL-6, IFN-γ, MIP-1α, IL-1β) were determined using ELISA as follows: Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm.

### Calculations

Percent Inhibition in each sample was calculated wrt control (LPS treated cells). [(B−A)/B]*100; Where A=Test Item treated cells, B=Control (LPS treated) cells.

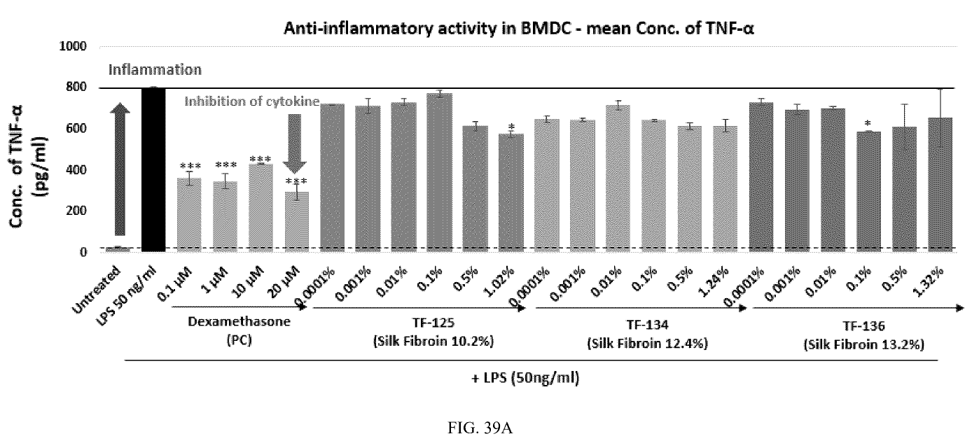

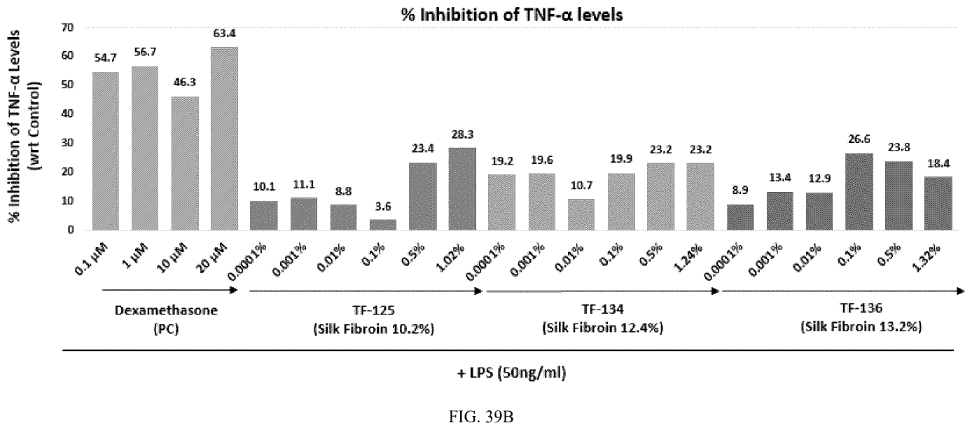

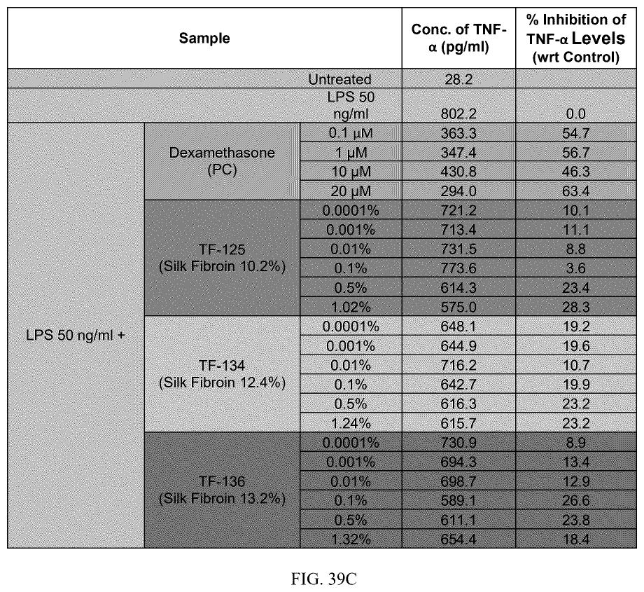

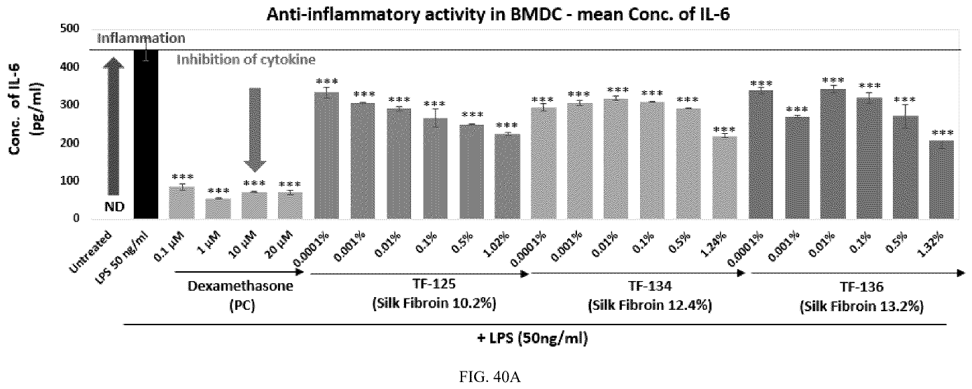

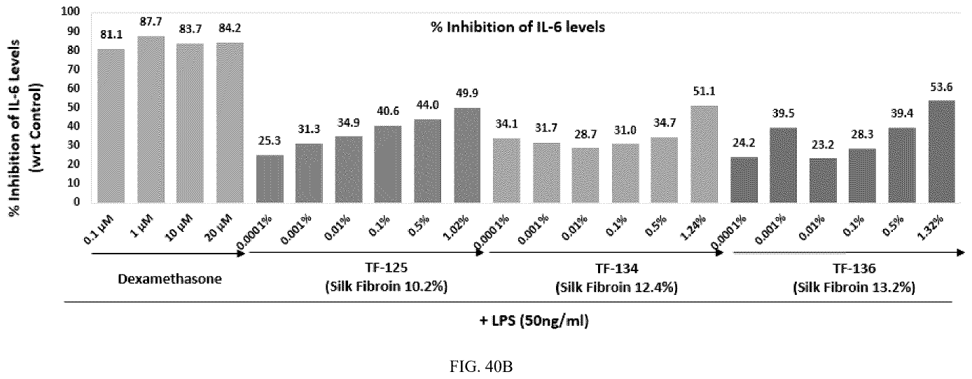

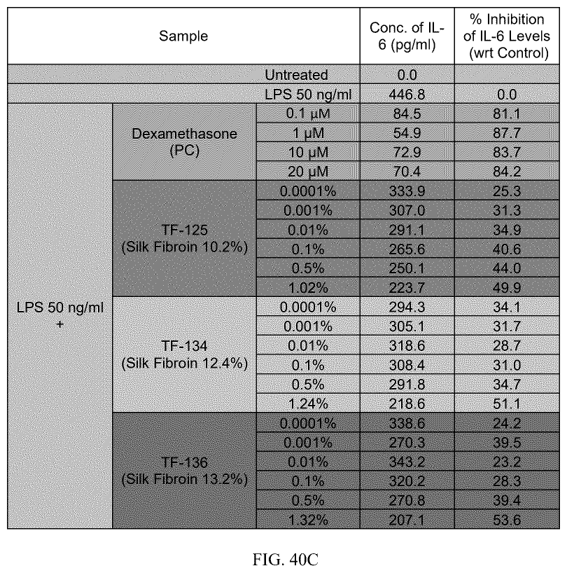

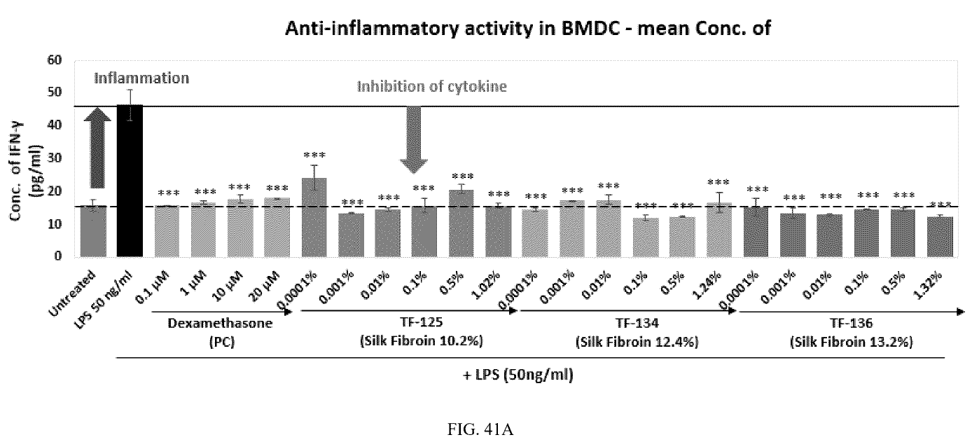

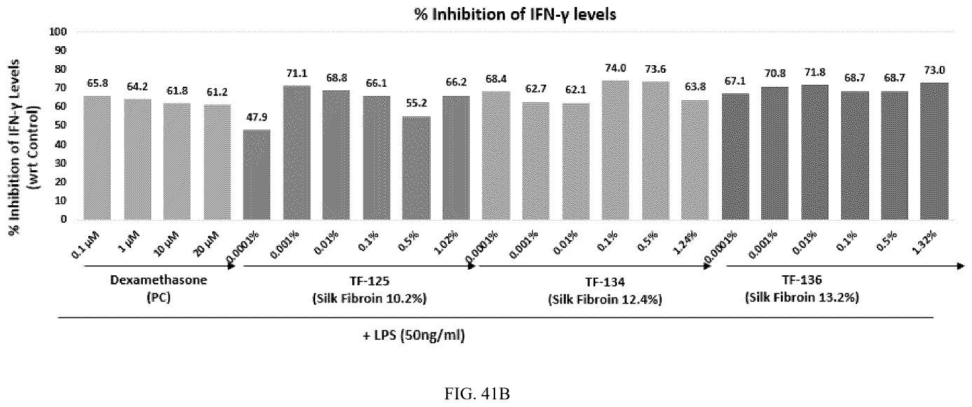

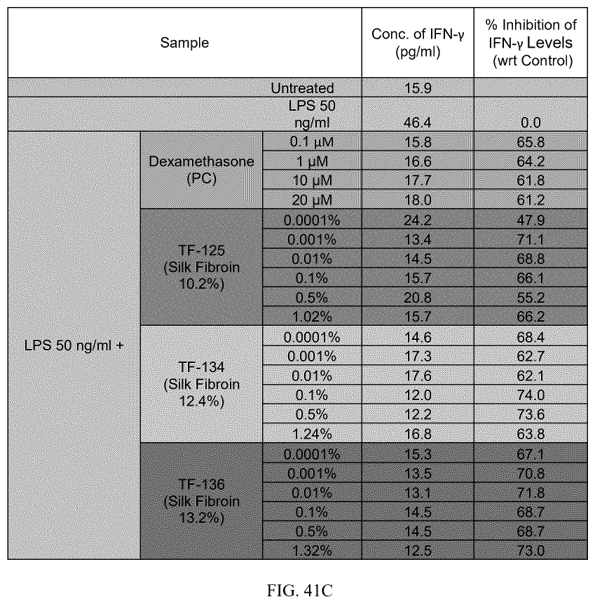

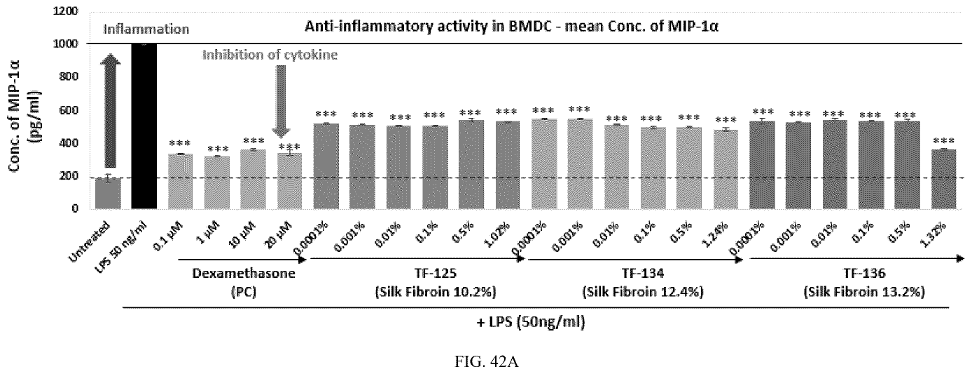

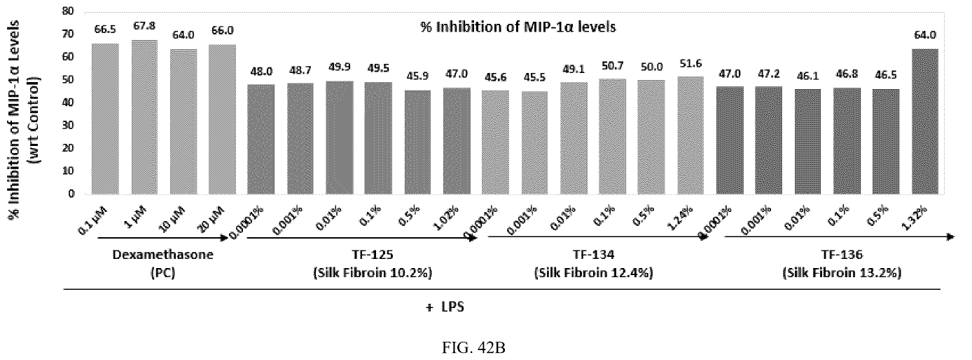

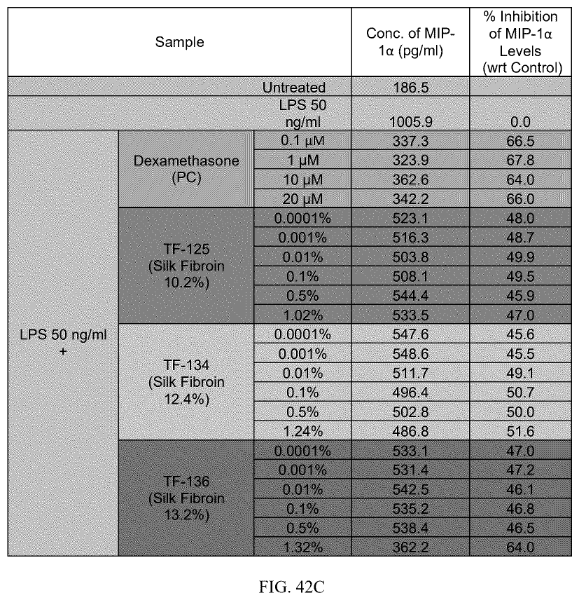

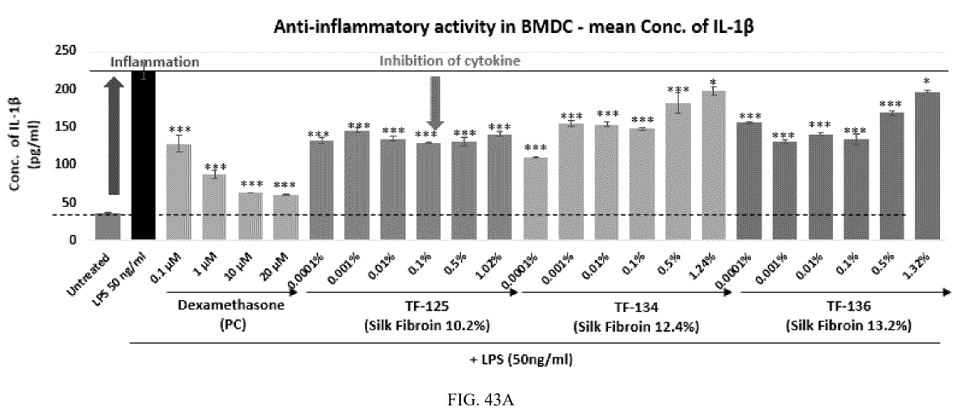

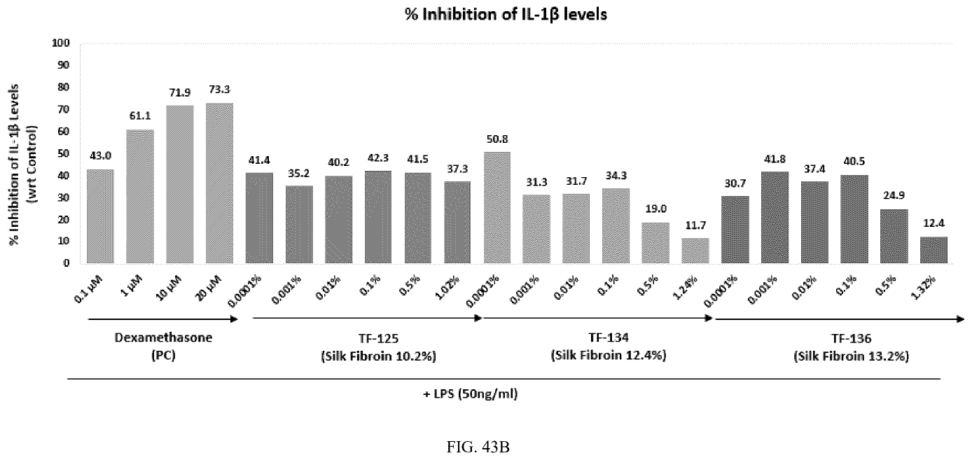

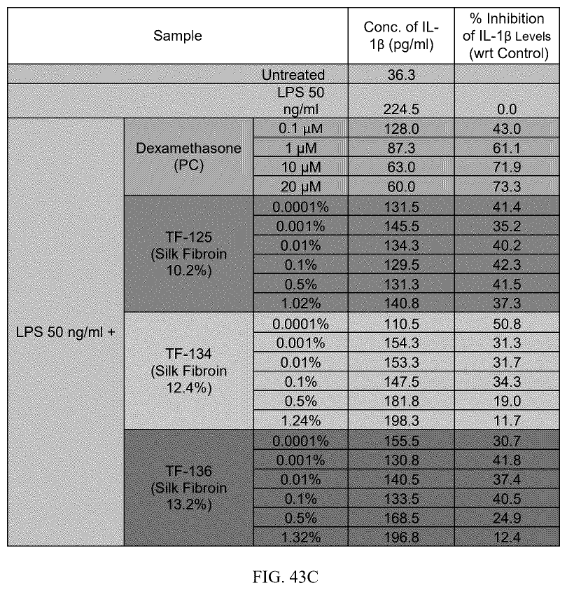

Results: seen at FIGS. 39-43.

### L. Anti-Inflammatory Activity—Inhibition of Cytokines in Human Monocytes (THP-1)

### Study Design

Test System—Human Monocytic Cells (THP-1)
Number of cells plated—0.1 million cells/well in 24 Well plate
FBS concentrations—0%,
Test items—TF-125 (10.2%), TF-134 (12.4%), TF-136 (13.2%)
Time points—24 h pre-treatment, 24 h stimulation,
Estimation method—Human TNF-α, IL-6, IL-8, MIP-1α, IL-1β ELISA.
Positive Control—Dexamethasone

### Procedure

Cells were plated in 10% FBS in 24-well plates and incubated for 24 hours. Cells were then serum starved in 0% FBS for 24 hours. Cells were treated with 3 Test items in 0% FBS at non-cytotoxic concentrations for 24 hours. After incubation, cells were stimulated with inflammatory stimulus (LPS 25 μg/ml). After 24 h of stimulation, culture supernatants were collected. Levels of cytokines (TNF-α,IL-6, IL-8, MIP-1α, IL-1β) were determined using ELISA as follows: Assay diluent was added to each well. Respective kit standards and samples (supernatants of cells) were directly pipetted into the wells and incubated for 2 h at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm.

### Calculations

Percent Inhibition in each sample was calculated wrt control (LPS treated cells). [(B−A)/B]*100; Where A=Test Item treated cells, B=Control (LPS treated) cells.

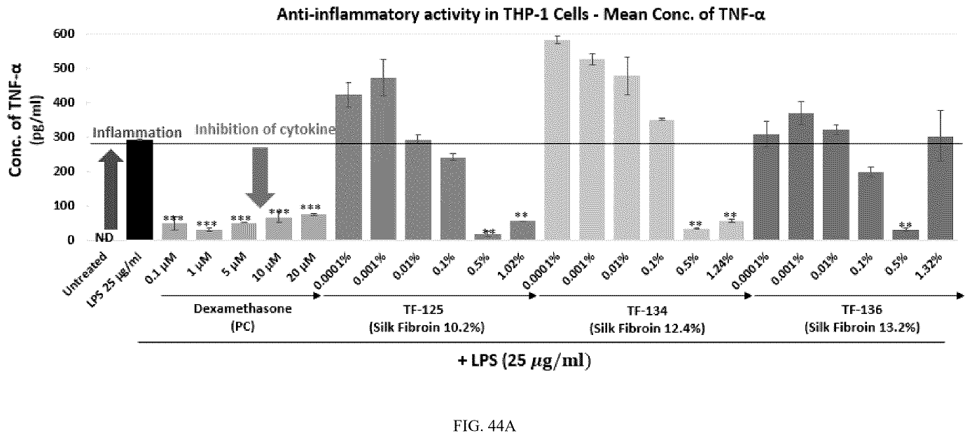

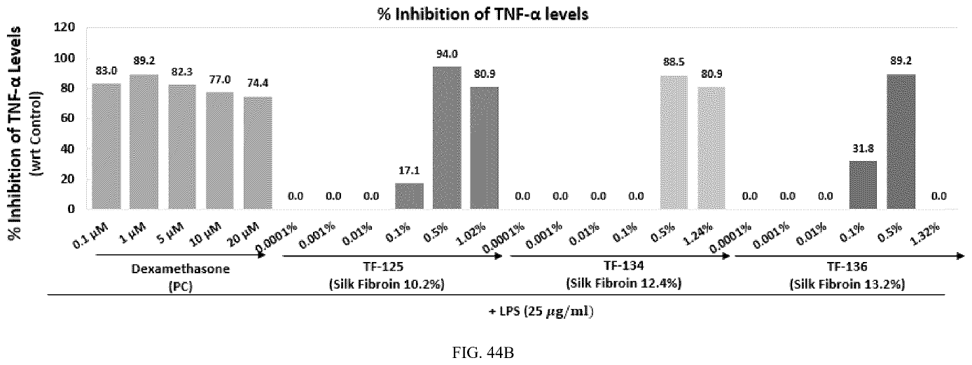

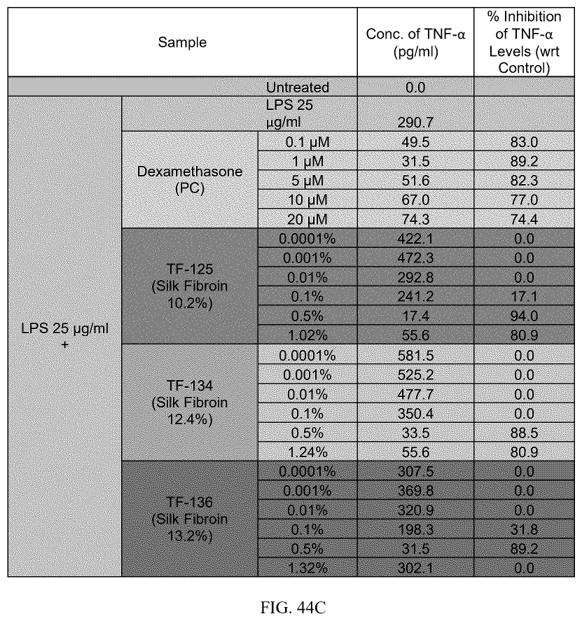

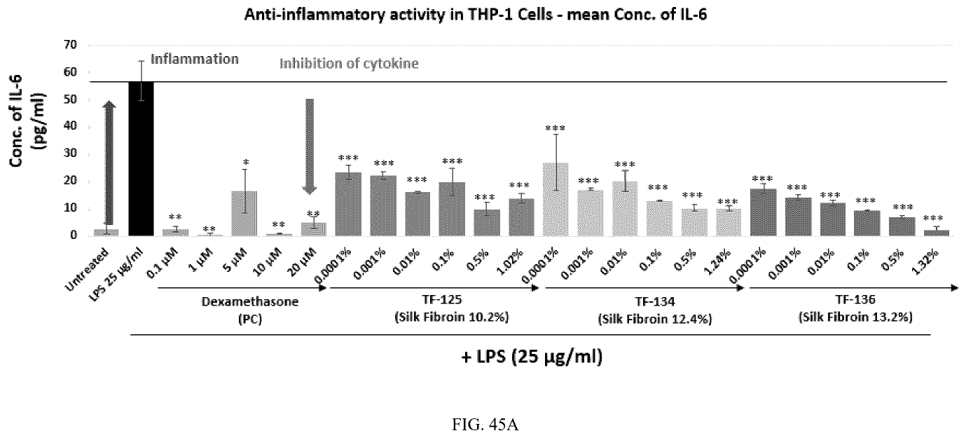

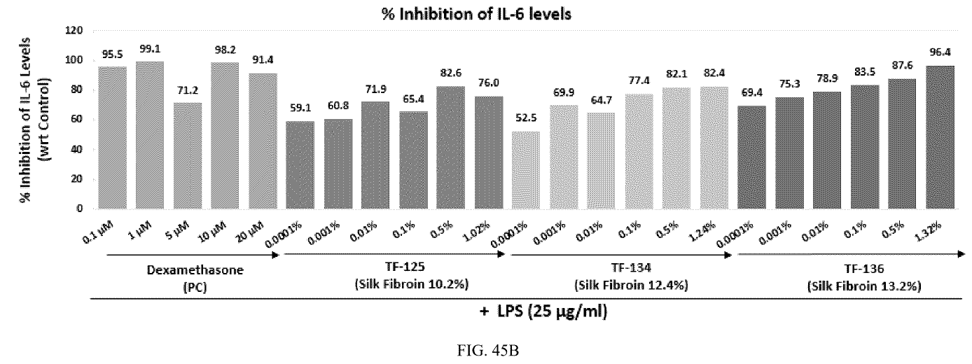

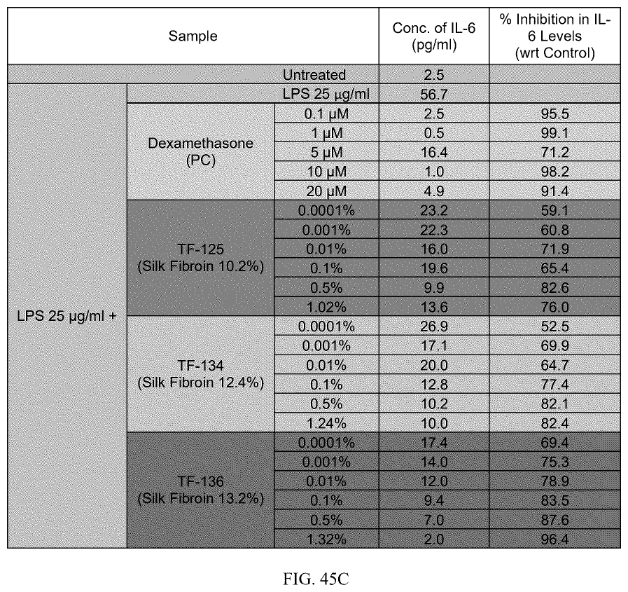

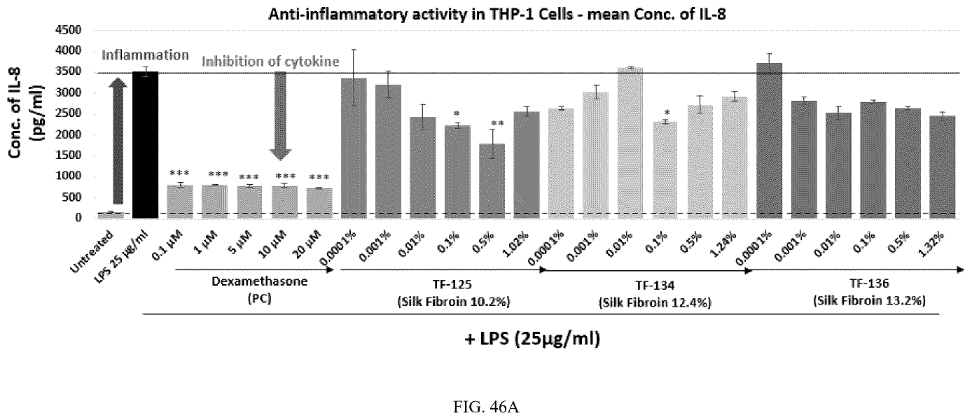

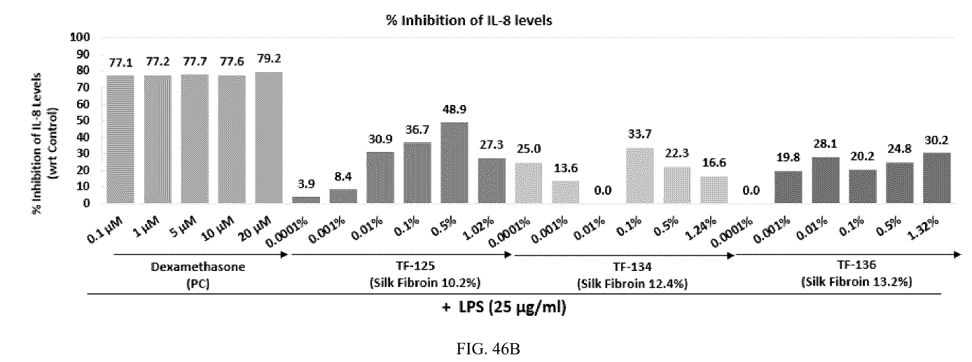

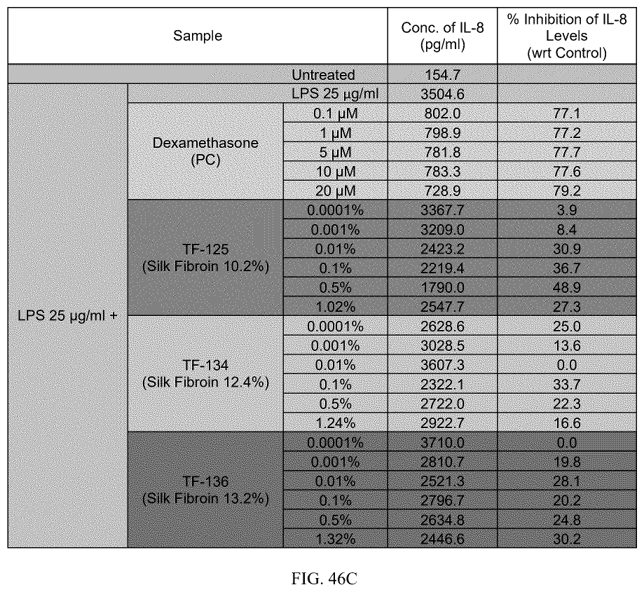

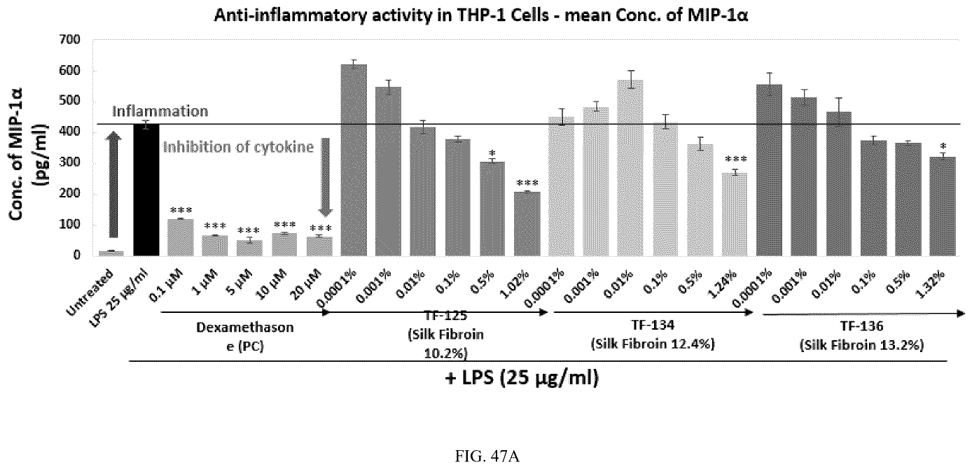

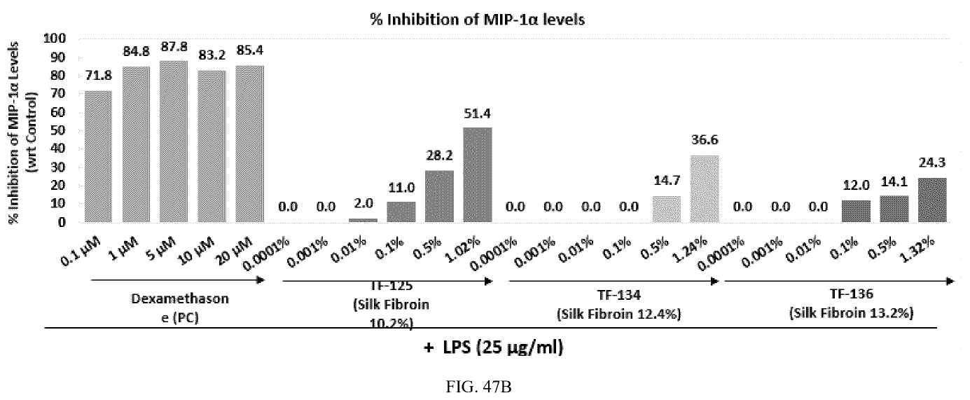

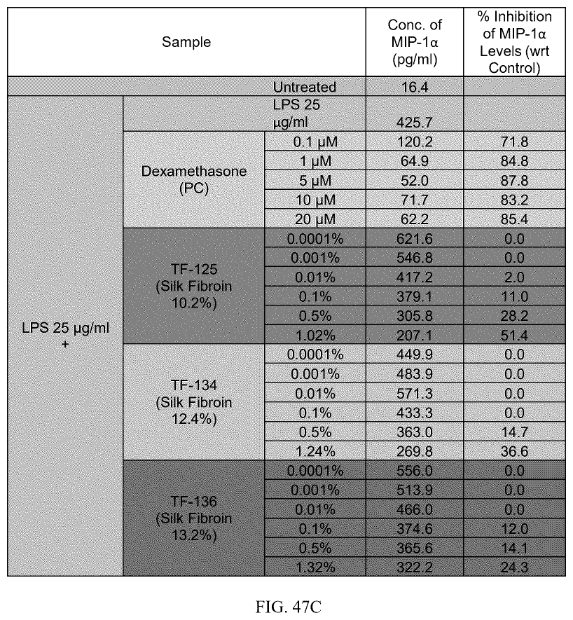

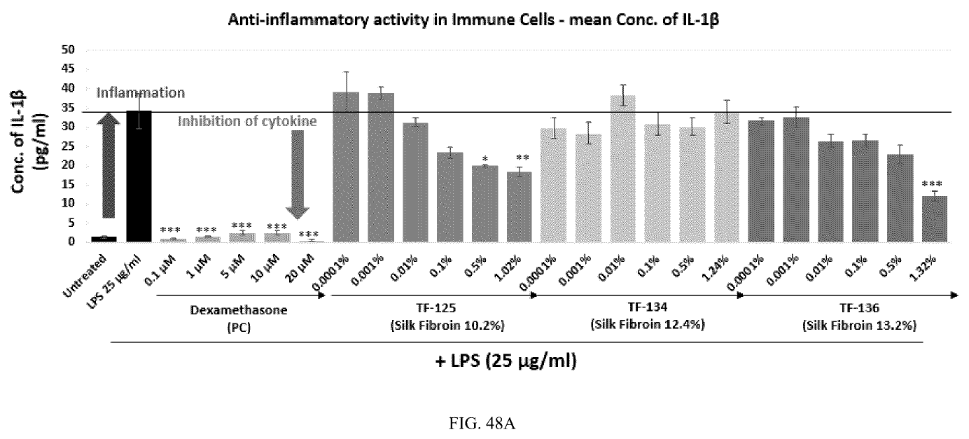

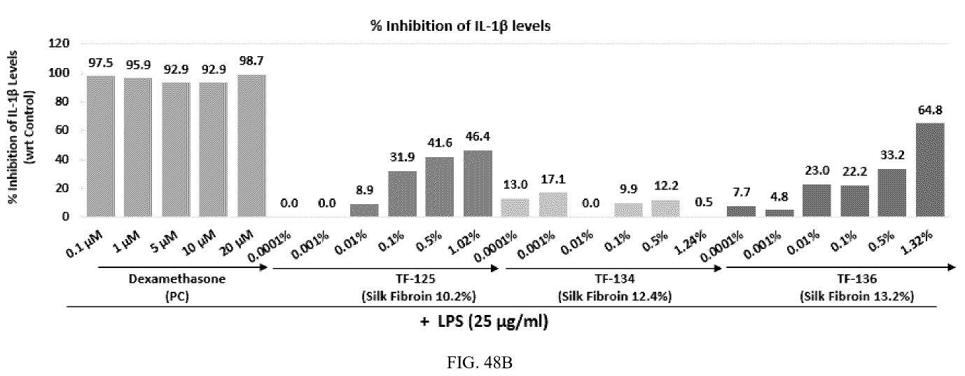

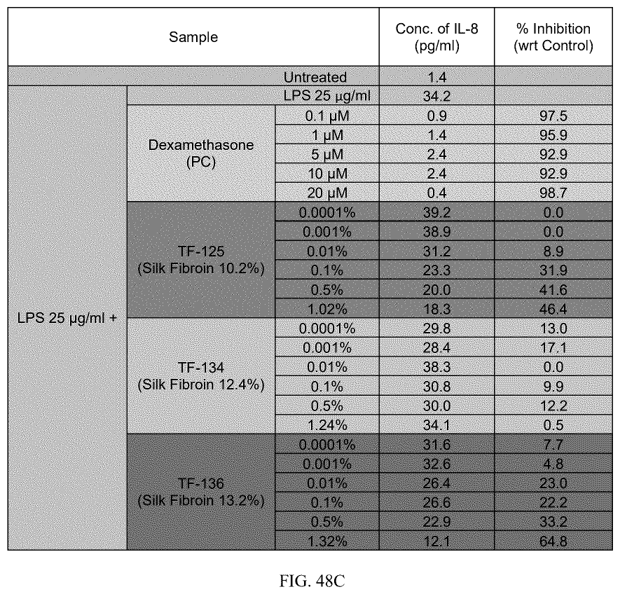

Results: seen at FIGS. 44-48.

Findings: The above data suggests that silk fibroin peptides according to the disclosure reduces inflammatory cytokine production in immune cells (mouse and human) that have been stimulated with LPS (inflammatory stimulus) suggesting that the silk ingredient could reduce systemic inflammation by reducing inflammation in immune cells.

It is to be understood that the words which have been used are words of description rather than limitation, and that changes may be made within the purview of the appended claims without departing from the true scope and spirit of the disclosure in its broader aspects.

While the present disclosure has been described at some length and with some particularly with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

What is claimed is:

1. A composition comprising fragments of silk fibroin peptides for use in the treatment of a disorder or condition associated with a digestive, pulmonary, or skin disorder, wherein the fragments of the silk-fibroin peptides have an average molecular weight of less than 90 kDa when measured by exclusion chromatography and less than 20 kDa when measured by dynamic light scattering.

2. The composition for use of claim 1, wherein the composition is derived from one or more natural sources, one or more synthetic sources, or combinations thereof.

3. The composition for use of claim 1, wherein the composition may be a hydrogel, a powder, a suspension, an emulsion, a solution, or combinations thereof.

4. The composition for use of claim 1, wherein the composition can be administered directly to the system of interest without significant or unintended degradation

5. The composition for use of claim 1, wherein the disorder or condition associated with a digestive disorder is a disorder of the upper or lower digestive system.

6. The composition for use of claim 5, wherein the composition targets the organs of the upper digestive system, such as the mouth, esophagus, and stomach, and is administered as a solution, hydrogel, emulsion, or suspension

7. The composition for use of claim 5, wherein the composition targets the organs of the lower digestive system, such as the small intestine, the large intestine, and the colon.

8. The composition for use of claim 7, wherein the composition is administered as a solution, a hydrogel, an emulsion, a suspension, or a powder, wherein said composition, when administered orally to be delivered post-stomach degradation, is in the form of enteric coated capsules, gel-caps, oil in water suspensions and tablets or in buffered solutions, or when administered rectally, is in the form of a suppository or enema

9. The composition for use of claim 1, wherein the composition will be formulated as a solution, hydrogel, emulsion, suspension, or powder that is systemically absorbed post-delivery to the intended zone of the digestive or pulmonary system.

10. The composition for use of claim 1, wherein the pulmonary system includes organs such as the nose, larynx, trachea, and/or lungs, and wherein the composition is be formulated as a solution, hydrogel, emulsion, suspension, or powder that is able to be aerosolized or nebulized for direct access to these tissues.

11. The composition for use of claim 1, wherein the composition may comprise or may be combined with one or more additional components selected from the group consisting of:
(a) a therapeutic agent;
(b) cargo;
(c) a microorganism; and
(d) a biological system.

12. The composition for use of claim 11, wherein the one or more additional components may be present in the composition by weight, volume, or concentration of from about 0.0001% to about to about 99.9%, or greater than 99.9%.

13. The composition for use of claim 11, wherein the one or more additional components may be present in the composition at a concentration of from about 0.01 pg/kg to about 10 mg/kg per kg of patient.

14. The composition for use of claim 1, wherein the composition may comprise silk fibroin peptides at a concentration between 0.1% and 100%.

15. The composition for use of claim 1, wherein the composition may be in powder form or in a solution which may be, but is not limited to, saline, phosphate buffer, borate buffer, and phosphate buffered saline

16. The composition for use of claim 1, wherein the silk fibroin peptides are presented in a salt form that may include but are not limited to: hydrochloride, sodium, sulfate, acetate, phosphate or diphosphate, chloride, potassium, Maleate, calcium, citrate, mesylate, nitrate, tartrate, aluminum, zinc, ammonium, and gluconate.

17. The composition for use of claim 1, wherein the composition is used in a therapeutically effective amount to treat respiratory disorders that include but are not limited to pneumonia, acute respiratory distress syndrome (ARDS), asthma, chronic obstructive pulmonary disease (COPD), SARS-CoV, SARS-CoV-2, MERS-CoV, rhinitis, bronchitis, emphysema, common cold, influenza, histoplasmosis, RSV, tuberculosis, whooping cough, autoimmune diseases (Lupus etc), cystic fibrosis, pulmonary sarcoidosis, for first aid uses after smoke inhalation, and as a prophylactic to prevent inflammation and damage.

18. The composition for use of claim 1, wherein the composition is used in a therapeutically effective amount to treat colorectal disorders that include but are not limited to: inflammatory bowel disease (IBD), Crohn's disease, ulcerative colitis, other autoimmune diseases, and other types of colitis e.g. microscopic colitis/lymphocytic colitis.

19. The composition for use of claim 1, wherein the composition is used in a therapeutically effective amount treat gastric disorders that include but are not limited to gastritis, bacterial colonization (Helicobacter pylori), NSAID side effects, and autoimmune diseases (autoimmune gastritis).

20. The composition for use of claim 1, wherein the composition is used in a therapeutically effective amount treat progressive endothelial dysfunction, platelet sludging, blood clots, stroke, CVD, cancer, diabetes mellitus, chronic kidney disease, non-alcoholic fatty liver disease, autoimmune disorders (Lupus etc), neurodegenerative disorders (Alzheimer's etc), sarcopenia, osteoporosis, immunosenescence, and chronic inflammatory systemic diseases (e.g. rheumatoid arthritis, multiple sclerosis).

21. The composition for use of claim 1, wherein the composition is used in a therapeutically effective amount treat asthma, COPD, and lung cancer.

22. The composition for use of claim 1, wherein the composition is used in a therapeutically effective amount treat alcoholic liver disease, non-alcoholic steatohepatitis and viral hepatitis.

23. The composition for use of claim 1, wherein composition is used in a therapeutically effective amount treat gastroduodenal ulcers, GI malignancies (cancers), and Inflammatory bowel disease (IBD).

24. The composition for use of claim 1, wherein the composition is used in a therapeutically effective amount treat infections that benefit from increased cytokine response, cancer, and immunodeficiency disorders.

25. The composition for use of claim 1, wherein the processed silk fibroin peptides are complexed with cyclodextrin, alpha-cyclodextrin, beta-cyclodextrin, hydroxypropyl-beta-cyclodextrin, sulfo-butyl-ether-beta-cyclodextrin, or the like.

26. The composition for use of claim 1, wherein the processed silk fibroin peptides are processed by degumming raw silk and further dissolving silk fibroin fibers following the degumming process.

27. A method of treating a disorder or condition associated with the digestive system, the pulmonary system, or the skin, in a subject in need thereof, comprising administering to the subject a therapeutically effective amount of a composition comprising fragments of silk-fibroin peptides having an average molecular weight of less than 90 kDa when measured by exclusion chromatography and less than 20 kDa when measured by dynamic light scattering, wherein the composition and the disorder or condition is as defined in claim 1.

28. Use of a composition comprising fragments of silk-fibroin peptides in the manufacture of a medicament for use in the treatment of a disorder or condition associated with the digestive system, the pulmonary system, or the skin, wherein the fragments of silk-fibroin peptides have an average molecular weight of less than 90 kDa when measured by exclusion chromatography and less than 20 kDa when measured by dynamic light scattering, and the composition and the disorder or condition is as defined in claim 1.

29. The composition of claim 1, wherein the silk-fibroin peptides have an average polydispersity of less than 1.4 as determined by dynamic light scattering.
